(19) United States
(12) Patent Application Publication
(10) Pub. No.: US 2023/0381378 A1
(43) Pub. Date: Nov. 30, 2023

(54) METHODS AND COMPOSITIONS FOR THE TREATMENT OF IMPLANT ASSOCIATED COMPLICATIONS

(71) Applicant: The Board of Trustees of the Leland Stanford Junior University, Stanford, CA (US)

(72) Inventors: Geoffrey C. Gurtner, Portola Valley, CA (US); Jagannath Padmanabhan, Redwood City, CA (US)

(21) Appl. No.: 18/029,619

(22) PCT Filed: Oct. 7, 2021
(86) PCT No.: PCT/US2021/053992

§ 371 Date: Mar. 30, 2023

Related U.S. Application Data
(60) Provisional application No. 63/088,953, filed on Oct. 7, 2020.

(51) Int. Cl. A61L 27/54; A61L 27/28; A61F 2/02

(52) U.S. Cl. CPC A61L 27/54; A61L 27/28; A61F 2/02; A61L 2300/606; A61L 2400/18; A61F 2250/0067; A61L 2300/432

(57) ABSTRACT

The present invention relates to the prevention and treatment of implant associated complications; e.g. foreign body response (FBR). High mechanical stress at the implant-tissue interface activates a sustained inflammatory response, which is primarily responsible for implant associated complications and implant failure. In particular, described herein are apparatuses, devices, and compositions containing inhibitors of mechanotransduction, and methods of making and using such apparatuses, devices and compositions. These apparatuses, devices, compositions, and methods are useful for preventing or reducing unwanted fibrosis, inflammation or cancer that result from implantation of a biomedical implant in an individual. One aspect of the invention provides a biomedical device including an effective amount of a composition containing a mechanotransduction inhibitor for preventing, inhibiting or treating implant associated complications.

### CROSS REFERENCE TO RELATED APPLICATION

The present application claims the benefit of and priority to U.S. Provisional Patent Application No. 63/088,953, filed Oct. 7, 2020, the entire disclosure of which is hereby.

### BACKGROUND

Biomedical implants have revolutionized modern medicine by improving the survival and the quality of life for millions of patients worldwide. Over 70 million devices, including breast implants, pacemakers, and orthopedic prostheses, are implanted globally each year and are associated with more than $100 billion in annual expenditure. However, chronic inflammation around implanted devices leads to reduced biocompatibility and results in the development of a long-term foreign body response (FBR). In clinical practice, the longevity of biomedical implants is limited by pathological FBR, frequently leading to implant failure and eventual rejection. Nearly 90% of all implant failures in commonly used medical devices are associated with FBR, and up to 30% of all implantable devices will undergo failure during their lifetime. As advances in materials science and electronics continue to shape the design of increasingly sophisticated biomedical devices, modifying the underlying host inflammatory response to these biomaterials remains the final frontier in developing truly biointegrative medical devices.

FBR begins as a wound healing-like response to the local tissue trauma that occurs during initial surgical implantation of a foreign device. Shortly thereafter, FBR begins a transition toward a longterm response state in which a fibrous capsule forms around the implant, leading to both device malfunction and distortion of surrounding tissue. The current prevailing hypothesis is that FBR is primarily a reaction of the local host tissue to the chemical and mechanical surface properties of the implanted material. Accordingly, recent research has focused on novel chemistries to identify rare, “superbiocompatible” materials such as zwitterionic hydrogels and triazole-containing alginates that appear to significantly reduce the FBR. Similarly, novel strategies for modulating the mechanical properties of biomaterials have also been developed, which show that soft materials can reduce fibrosis, although they do not reduce inflammation. While these developments have significantly improved our understanding of FBR and may promote novel biomaterial strategies, there are limitations to this approach. Hydrogels and other soft materials have a low range of elastic moduli (1-100 KPa) and cannot be used for biomedical devices that need to provide structural support (e.g., bone and orthopedic implants) or devices that interact with relatively stiffer tissues (e.g., pacemakers and neurostimulators). Thus, the vast majority of commonly used biomedical devices continue to be fabricated from traditional materials such as silicone and titanium and are therefore subject to high rates of FBR-related implant failure.

Hence there is a critical need for therapies to improve outcomes after biomedical device implantation. Described herein are therapies for improving outcomes after biomedical device implantation.

### SUMMARY OF THE DISCLOSURE

The present invention relates to the prevention and treatment of implant associated complications; e.g. foreign body response (FBR). High mechanical stress at the implant-tissue interface activates a sustained inflammatory response, which is primarily responsible for implant associated complications and implant failure. In particular, described herein are apparatuses, devices, and compositions containing inhibitors of mechanotransduction, and methods of making and using such apparatuses, devices and compositions. These apparatuses, devices, compositions, and methods are useful for preventing or reducing unwanted fibrosis, inflammation or cancer that result from implantation of a biomedical implant in an individual. One aspect of the invention provides a biomedical device including an effective amount of a composition containing a mechanotransduction inhibitor for preventing, inhibiting or treating implant associated complications. Another aspect of the invention provides a biomedical device including an effective amount of a composition containing a mechanotransduction inhibitor and a secondary treatment modality for preventing, inhibiting or treating implant associated complications.

Also provided is a small animal model of human-like FBR, which is created by altering the mechanical forces at the implant-tissue interface. The development of efficient therapeutic approaches to target FBR has been limited by a lack of readily translatable models. Although large animals more closely mirror human FBR physiology, mice are attractive because of the availability of transgenic strains. The small animal model disclosed herein overcomes these challenges and provides a novel model to tractably study human-like FBR in a small animal model.

Any of these biomedical devices can comprise an implant configured to prevent, inhibit, or treat implant associated complications when the biomedical device is implanted into a body of an individual having or at risk of having implant associated complications. Implant associated complication that benefit from the apparatuses, devices, compositions, and methods herein include, without limitation, foreign body response (FBR), capsular fibrosis, inflammation at or near the site of implantation, cancer (e.g. breast implant associated anaplastic large cell lymphoma), etc. Implant associated complications can be associated with a biomedical implant, such as, cosmetic implants (e.g. breast implants, buttock implants, calf implants, chin implants, nasal implants, etc.) ocular implants (e.g. lens implantation associated with cataracts surgery), auditory implants (e.g. cochlear implants), cardiac pacemakers, neurostimulators, orthopedic hardware, contraceptive implants, drug delivery device implants, etc.

The implant or other biomedical device can be configured to stay in the body at least 2 weeks, at least 4 weeks, at least 8 weeks, at least 3 months, at least 6 months, or at least one year.

These or other biomedical devices can include an insert configured to prevent, inhibit, or treat implant associated complications when the biomedical device is inserted into a body of an individual having or at risk of having implant associated complications. In any of these or other biomedical devices, the biomedical implant can comprise an interface configured to prevent, inhibit, or treat capsular fibrosis when placed on a body of an individual having or at risk of having capsular fibrosis.

In any of these or other biomedical devices, the biomedical implant can be configured to administer the composition systemically. In any of these or other biomedical devices, the biomedical implant can be configured to administer the composition locally.

In any of these or other biomedical devices, the biomedical implant can be configured to administer the composition intraarterially, intraarticularly, intramuscularly, intraocularally, intraperitoneally, intravenously, or subcutaneously. In these or other biomedical devices, the biomedical implant can be configured to administer the composition buccally, intranasally, orally, intrarectally, intravaginally, or sublingually. In these or other biomedical devices, the biomedical implant can be configured to administer the composition iontophoretically, topically, or transdermally.

In these or other biomedical devices, the biomedical implant can be coated with the composition. In these or other biomedical devices, the biomedical implant can be impregnated with the composition. In these or other biomedical devices, the composition can be covalently attached to the device. In these or other biomedical devices, the composition can be non-covalently attached to the device. In these or other biomedical devices, the biomedical device can comprise a silicone implant.

Yet another aspect of the invention provides a method of preventing, inhibiting, or treating implant associated complications in an individual in need thereof, comprising the steps of selecting an individual that has or is at risk of having implant associated complications; and administering to the individual a composition having an effective amount of a mechanotransduction inhibitor and thereby preventing, inhibiting, or treating implant associated complication in the individual. Mechanotransduction inhibitors that find use in present invention include, without limitation, inhibitors of RAC1, RAC2, CCL4, GADD45A, IQGAP1, e.g. human RAC1, RAC2, CCL4, GADD45A, IQGAP1. When a RAC1 inhibitor is used, the RAC1 inhibitor may include, without limitation, NSC23766, ZINC69391, IA-166, CAS 1177865-17-6, etc. When a RAC2 inhibitor is used, the RAC2 inhibitor may include, without limitation, NSC23766, EHT 1684, etc.

Yet another aspect of the invention provides a method of preventing, inhibiting, or treating implant associated complications in an individual in need thereof comprising the steps of selecting an individual that has or is at risk of having implant associated complications; and administering to the individual a composition having an effective amount of a monoterpenoid in addition to the mechanotransduction inhibitor and thereby preventing, inhibiting, or treating capsular fibrosis in the individual.

Yet another aspect of the invention provides a method of preventing, inhibiting, or treating implant associated complications in an individual in need thereof comprising the steps of selecting an individual that has or is at risk of having implant associated complications; and administering to the individual a composition having an effective amount of a mechanotranduction inhibitor and thereby preventing, inhibiting, or treating implant associated complications in the individual. Yet another aspect of the invention provides a method of preventing, inhibiting, or treating implant associated complications in an individual in need thereof comprising the steps of selecting an individual that has or is at risk of having implant associated complications; and administering to the individual a composition having an effective amount of macrophage inhibiting activity in addition to the mechanotransduction inhibitor and thereby preventing, inhibiting, or treating capsular fibrosis in the individual. Another aspect of the invention provides a method of preventing, inhibiting, or treating implant associated complications in an individual in need thereof comprising the steps of selecting an individual that has or is at risk of having implant associated complications; and administering to the individual a composition having an effective amount of myeloid cell, lymphoid cell or fibroblast inhibiting activity in addition to the mechanotransduction inhibitor and thereby preventing, inhibiting, or treating capsular fibrosis in the individual.

In these or any other methods the composition can be administered intraarterially, intraarticularly, intramuscularly, intraocularally, intraperitoneally, intravenously, or subcutaneously. In these or any other methods the composition can be administered buccally, intranasally, orally, intrarectally, intravaginally, or sublingually. In these or any other methods the composition can be administered iontophoretically, topically, or transdermally. Administration can be localized to the site of the implant.

In these or any other methods the composition can be administered locally at the implant site. In these or any other methods the composition can be administered systemically.

In these or any other methods the administering step includes implanting an implant including the composition.

In these or any other methods the implant is further configured to treat a medical issue or cosmetic issue other than fibrosis.

In these or any other methods the administering step includes inserting an insert including the composition. In these or any other methods the administering step includes placing a skin interface of a biomedical device on a skin of the individual.

In these or any other methods the composition prevents, inhibits, or reduces macrophage activity or differentiation at least 10%, at least 20%, at least 30%, at least 40% or at least 50%. In these or any other methods the composition prevents, inhibits, or reduces Arg1⁺ macrophage activity or differentiation at least 10%, at least 20%, at least 30%, at least 40% or at least 50%. In these or any other methods the composition prevents, inhibits, or reduces lymphocyte activity or differentiation at least 10%, at least 20%, at least 30%, at least 40% or at least 50%. In these or any other methods the composition prevents, inhibits, or reduces fibroblast activity or differentiation, fusogenic macrophage activity or differentiation at least 10%, at least 20%, at least 30%, at least 40% or at least 50% and/or foreign body giant cell activity or differentiation at least 10%, at least 20%, at least 30%, at least 40% or at least 50%.

In these or any other methods the composition prevents, inhibits, or treats collagen deposition. In these or any other methods the composition prevents, inhibits, or treats a foreign body response.

In these or any other methods the composition the individual is human.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1. Pathological FBR in humans is mediated by RAC2 mechanotransduction signaling, regardless of implant properties and is associated with increased mechanical signaling. (A) Schematic of various implant types. (B) Trichrome staining of fibrotic capsules from the fibrous capsule formed around silicone-based breast implants, titanium-based pacemakers and stainless steel-based orthopedic implants are all strikingly similar to one another. n=4-6 for each implant category. (C,D) Quantification of collagen and mature collagen shows no significant differences between the different types of human implants. (E) Schematic showing the experimental methodology followed; FBR capsules from Baker I and Baker IV breast implants were subject to molecular analyses using a commercially available biomarker panel (HTG Molecular). A total of 9 Baker I specimen and 11 Baker IV specimen were used in this study. (F) Heatmap of the top 100 genes upregulated in Baker IV vs. Baker I breast implants, organized in decreasing order of fold change from left to right. (G) Pathways significantly upregulated in Baker IV samples analyzed using Database for Annotation, Visualization and Integrated Discovery (DAVID). Pathways highlighted in red are mechanical signaling pathways and those highlighted in green are inflammatory pathways. (H) STRING (Search Tool for the Retrieval of Interacting Genes/Proteins) analysis showing that Rac2 is a central mediator of both mechanotransduction signaling as well as inflammatory signaling genes that were all upregulated in Baker IV specimen. STRING analyzed interactions between the different genes based on experimental evidence as well as the predicted interactions based on various databases, which are color-coded as listed below: pink (experimentally determined), blue (curated databases), green (gene neighbourhood), red (gene fusion), blue (gene co-currence), light green (textmining), black (co-expression), violet (protein homology).

FIG. 2. Altering tissue-scale forces around implants using mechanically stimulating implants (MSIs) produces human-like FBR capsule architecture in mice. (A) Finite element modeling of murine and human implants showing that human implants are subject to 100-fold higher mechanical stress than murine implants. (B) Schematic and picture of the Mechanically Stimulating Implant (MSI) model in mice. FE modeling confirming that MSI recreate human-like mechanical stress in the mouse. (C) Trichrome staining of FBR capsules in the human implant capsules, standard murine model, and the MSI murine model. Scale Bar=50 μm. (D) Herovici staining of FBR capsules in the the human implant capsules, standard murine model, and the MSI murine model showing mature (red) and immature (blue) collagen. Scale bar=50 μm. (E) Immunostaining for alpha smooth muscle actin (aSMA, third row), a marker for myofibroblasts, of FBR capsules in the human implant capsules, standard murine model, and the MSI murine model. Scale bar=50 μm. (F) Scanning electron microscopy (SEM) imaging of the surface of the capsules in the the human implant capsules, standard murine model, and the MSI murine model. Scale bar=10 μm. (G) (Top row) Quantification of percent area positive for collagen in each capsule (far right column). n=5 for each group. *p<0.05. (Second row) Quantification of mature collagen deposition in the FBR tissue (far right). n=5 for each group. *p<0.05. (Third row) Quantification of aSMA normalized to cell density using image analyses in each capsule. (Fourth row) Quantification of surface collagen percent area associated with each capsule. n=8 images for each group. *p<0.05.

FIG. 3. Mechanically stimulated implants display a sustained inflammatory response at the implant-tissue interface. (A) UMAP plots of all cells from murine FBR capsules classified by sample type and timepoints. A total of 36,827 cells were analyzed. (B) MSI capsules show a robust activation of Rac2 and associated inflammatory markers as compared to standard implant capsules. (C) Heatmap of differentially regulated inflammatory genes between standard implant cells and MSI cells, showing a similar pattern of expression. All inflammatory genes analyzed are upregulated in MSI capsules at both early and late timepoints. In contrast, most inflammatory markers show a modest activation in standard implants at the early timepoint, which subsides at the late timepoint. (D) UMAP plots of all cells from murine FBR capsules classified by Seurat clusters. Two major cell types were found in both standard implants and MSIs: immune cells (myeloid & lymphoids) and fibroblasts. (E) Relative proportion of myeloid, lymphoid and fibroblast cells in standard murine implants and MSI capsules. Myeloid cells were the most abundant cell type in both capsules and were especially enriched with mechanical stimulation.

FIG. 4. Increased tissue-scale forces activate Rac2 signaling in myeloid cells, which drives the Baker IV fibrotic phenotype in mice. (A,B) UMAP Plot of myeloid cells in SM and MSI implant capsules. Clusters 1,4 and 7 are highly enriched in MSI capsules. (C) MSI myeloid cell clusters (Cluster 1, 4 & 7) show significant upregulation of Baker IV markers (FIG. 1B), including key mechanotransduction and inflammatory chemokine signaling pathways. (D) In contrast, SM implant clusters upregulate Baker I markers. (E) Baker IV markers including Rac2 and downstream mechanotransduction and inflammatory genes were differentially upregulated in the MSI clusters. (F) Pathways significantly upregulated in Baker IV samples analyzed using Database for Annotation, Visualization and Integrated Discovery (DAVID). Pathways highlighted in red are also upregulated in Baker IV human specimen.

FIG. 5. Increasing tissue-scale forces activates fusogenic macrophages, MHC II lymphocytes and myofibroblasts, all classic features of pathologic FBR. (A) Relative proportions of standard implant cells and MSI cells in Cluster 4 cells, which are highly enriched in MSI samples. Cluster 4 cells upregulate markers for fusogenic macrophages. (B) UMAP Plot of lymphocytes from murine FBR capsules. MSI lymphocytes show upregulation of MHC Class II signaling. (C) UMAP Plot of fibroblasts from murine FBR capsules. Standard murine (SM) model fibroblasts show upregulation of proteolysis and signaling associated with negative regulation of cell proliferation, indicative of a resolved wound. MSI fibroblasts show upregulation of fibrotic markers. (D) Expression of mechanotransduction markers in SM and MSI fibroblasts.

FIG. 6. Blocking Rac2 signaling effectively reverses the human-like FBR induced by increased tissue-scale forces in mice. Comparative analysis of histology sections of FBR capsules from the MSI mouse model with and without the Rac inhibitor. (A) Immunostaining for Rac2 signaling in FBR capsules. Scale bar=50 μm. Quantification of percent area positive for Rac2 in each capsule. n=5 for each group. *p<0.05. (B) Immunostaining for aSMA signaling in FBR capsules. Scale bar=50 μm. Quantification of percent area positive for aSMA in each capsule. n=5 for each group. *p<0.05. (C) Trichrome staining of FBR capsules. Scale bar=50 μm. Quantification of percent area positive for collagen in each capsule. n=5 for each group. *p<0.05. (D) H&E staining of FBR capsules. Scale bar=50 μm. Quantification of average capsule thickness. n=4 for each group. *p<0.05. (E) In standard murine (SM) implants, there is a modest activation of inflammatory pathways at the early timepoint, which subsides at the late timepoint, resulting in minimal FBR. In contrast, MSI capsules, increased tissue-scale forces lead to the activation of Rac2 mechanical signaling, which promotes a robust activation of inflammatory markers that is sustained over time, resulting in a human-like pathological FBR.

FIG. 7. Pathological FBR in humans is characterized by similar fibrotic encapsulation, regardless of implant properties (A) Haematoxylin and eosin stain, (B) Trichrome staining and (C) Herovici staining of fibrotic capsules from the fibrous capsule formed around siliconebased breast implants, titanium-based pacemakers and stainless steel-based orthopedic implants are all strikingly similar to each other.

FIG. 8. Pathological FBR in humans in mediated by Rac2 mechanotransduction signaling. (A) Mechanotransduction (red) genes upregulated in Baker IV capsules. (B) Inflammatory genes (green) upregulated in Baker IV capsules. (C) Pathways significantly upregulated in Baker I samples analyzed using Database for Annotation, Visualization and Integrated Discovery (DAVID).

FIG. 9. Geometrical model and parameters used for FE modeling of mechanical stress around biomedical implants. (A,B) Geometrical model used for FE modeling of biomedical implants. (C) Average values for skin and subcutaneous tissue properties used for modeling based on previous literature.

FIG. 10. Human implants are subject to increased mechanical stress as compared to standard murine implants. (A) Schematic standard murine implants and and three commonly used human implants, breast tissue expanders, pacemakers and neurostimulator batteries. (B) FE modeling of standard murine models of FBR reveals minimal mechanical stress at the implant-tissue interface. FE modeling reveals high mechanical stress around human implants including breast implants, pacemakers and neurostimulator batteries.

FIG. 11. Development of mechanically stimulated implants (MSI) (A) To create human levels of mechanical stress in a mouse, we developed silicone implants with an encapsulated prefabricated coin motor, capable of in situ vibration. (B) To enable in situ vibration of MSIs, the wires from the implant had to be guided through the skin, which required a novel surgical technique (FIG. 3A). After skin incision and creation of a subcutaneous pocket on the back of the mice, two 20 G cannulas were inserted into the pocket in a cranio-caudal direction. The wires were tunneled through the pocket and guided through the skin using the cannulas and a modified Seldinger technique, enabling activation of the motor by an external battery.

FIG. 12. Mechanical testing of silicone implants. The Young's Modulus (Ey) of both the MSI and standard murine implants were determined using a custom compressive test method on Instron 5560. Each sample had a diameter of 15.6 cm and subjected to a compressive rate of 1 mm/sec. Ey of each implant was calculated by taking the linear slope of the stress-strain curve between 0 and 0.10 compressive strain.

FIG. 13. Timeline of vibration in vibration-enabled devices. MS's were vibrated daily for 1 hour from day 4 to day 12. The timeline for applied mechanical stress was based on our previous studies and the vibration power was chosen to resemble human conditions.

FIG. 14. Increased tissue-scale forces result in increased fibrosis around implants in mice, independent of implant chemistry or mechanical properties. (A,B) Trichrome staining and Herovici staining of FBR capsules formed around standard murine implants with low stiffness, standard implants with high stiffness, and MS's reveals that MSI-model produces a more robust scar tissue, with increased collagen and mature collagen. Scale bar=500 μm.

FIG. 15. ScRNAseq of cells from SM and MSI implant capsules (A) In standard murine (SM) implants, there is a modest activation of inflammatory pathways at the early timepoint, which subsides at the late timepoint. In contrast, MSI capsules show a robust activation of inflammatory markers that is sustained over time. (B) Heatmap of differentially regulated genes in myeloid cells. MSI myeloid cells upregulated inflammatory markers.

FIG. 16. (A) Differentially upregulated genes between lymphocytes cells from the standard murine implants and MS's.

FIG. 17. (A) Differentially upregulated genes between fibroblast cells from the standard murine implants and MS's.

FIG. 18. A unique clinical case provides insights into the role of mechanotransduction signaling in the prolonged inflammatory response to textured implants. (A) Intraoperative photographs. Left: Internal capsule adherent to textured implant surface after opening the capsule on the back table. This capsule has been incised vertically and forceps inserted to demonstrate the tight adherence. Center: The internal capsule covered all textured surfaces but spared the smooth central area on the deep surface of the implant and the three orientation knobs. Right: View of the external capsule, adherent to native subcutaneous tissue, through the wound (superior). (B) Top: H&E of internal capsule showing unidirectionally aligned fibers with increased cellularity. Center: Trichrome stain demonstrating increased collagen deposition in the internal capsule as compared to the external capsule. Bottom: Herovici's stain demonstrating increased deposition of mature, red collagen staining in the internal capsule. Scale bars=100 μm. p<0.05. (C) Top: Picrosirius red staining demonstrating the internal capsule with highly aligned and tightly packed collagen fibers, whereas the external capsule shows a disorganized network, consistent with the typical histological presentation of EDS. Center: Immunofluorescence staining of the internal capsule shows a higher activation of α-SMA positive (green) myofibroblasts as compared to external capsule. Bottom: Immunofluorescence staining shows increased expression of cytokine MCP1 (red) in the internal capsule, indicating a highly inflammatory environment. Optical fields were analyzed from 6 tissue sections of our case. Scale bars=100 μm. p<0.05. (D) The presence of a textured breast implant creates adhesion and subsequently a high mechanical stress environment leading to myofibroblast activation and deposition of collagen in a highly organized pattern. In contrast, the external non-adherent capsule, under low mechanical stress, forms in a manner more consistent with EDS. The mechanical activation of inflammatory signaling protein MCP-1 in the adherent/internal capsule provides a direct mechanism for breast-implant associated inflammation, which plays a significant role in the pathogenesis of BIA-ALCL.

FIG. 19. IQGAP1 serves as a central mediator of mechanical signaling in human FBR. (A) Schematic and gross pictures of explanted biomedical devices and associated FBR capsules from human pacemakers and neurostimulator batteries, submitted with respective control subcutaneous tissue for mass spectrometry protein analysis. (B) Heatmap of the top 25 proteins that are expressed in peri-prosthetic human capsular tissue and top 25 proteins expressed in human subcutaneous tissue, organized in decreasing order of fold change. (C) Top upregulated protein pathways in human FBR capsules were identified using STRING pathway analyses. (D) Schematic showing the role of IQGAP1 as a scaffolding protein in various major mechanotransduction pathways.

FIG. 20. Activation of IQGAP1 mediated mechanical signaling pathways is confirmed in murine FBR. (A) Schematic of MSI mice and associated FBR capsule submitted with control mice subcutaneous tissue for mass spectrometry protein analysis. (B) Heatmap of top 25 proteins expressed in explanted MSI mouse capsule tissue and top 25 proteins expressed in control mouse subcutaneous tissue, organized in decreasing order of fold change. (C, D) Top upregulated protein pathways in murine MSI fibrous capsules identified via STRING pathway analyses, with IQGAP1 being identified as a common mediator of top mechanical and immune cell signaling pathways.

FIG. 21. IQGAP1 deficient and WT MSI mice capsule tissue histology and scRNA-seq. (A) Schematic of IQGAP1+/− and WT mice and associated FBR capsules submitted for histological analysis and scRNA-seq. (B) Trichrome staining (first column) of FBR capsules formed from MSI-WT and MSI-IQGAP1+/− mouse models reveals that knockout mice have decreased collagen deposition. Scale bar=50 μm. Quantification of percent are positive for collagen shows that the IQGAP1 deficient MSI-mice displays decreased collagen deposition (n=4 for each group. *p<0.05). Herovici staining (second column) of tissue sections reveal that IQGAP deficient MSI-mice exhibit decreased total mature (red) collagen deposition (n=4 for each group. *p<0.05). Scale bar=50 μm. (C, D) Cells that populate MSI-FBR capsules in WT and IQGAP1 deficient mice were analyzed using 10× single cell RNA sequencing (scRNA-seq). UMAP plot for scRNA-seq data identifying the 6 unique clusters of cells, encompassing endothelial cells, monocytes/macrophages, vascular smooth muscle cells (VSMCs), fibroblasts, T cells, and neutrophils, identified based on differential transcriptional profiles.

FIG. 22. IQGAP1 deficient mice show diminished mechanical signaling, inflammation, and fibrosis. (A) UMAP plot for scRNA-seq data with cells colored according to the source (WT or IQGAP1+/− tissue). (B) Violin plots showing gene expression of Thbs1, Ccl3, II1b, Rac1, Ccl4, Fn1, Hmox1, Lyz1, and Crip1 in the WT and IQGAP1+/− myeloid cell clusters. (C) Violin plots showing gene expression of Acta2, Cxcl5, Cxcl2, Saa3, Ptx3, Cd9, Apoe, Tppp3, and Clec3b in the WT and IQGAP1+/− fibroblast clusters. (D) Immunostaining of myofibroblast marker a-SMA (first column) and mechanotransduction mediator p-cdc42/Rac1 (second column) in FBR capsules from WT and IQGAP1+/− mice (n=4 for each group. *p<0.05). Scale bar=50 μm.

FIG. 23. (A) Photographs of the MSI implant ex vivo and in situ (b) Relative number of cells from WT and KO mice in each cluster identified by single cell RNA Sequencing.

### DETAILED DESCRIPTION

### Definitions

Implant associated complications. Implant associated complications refer to any complication that can be associated with the placement of an implant and the effects of the implant on the local environment. Complications can include foreign body response (FBR), fibrosis, capsular fibrosis, inflammation at or near the site of implantation, cancer (e.g. breast implant associated anaplastic large cell lymphoma).

Fibrosis. Fibrosis is the formation of excess fibrous connective tissue, such as overgrowth, hardening and/or scarring of connective tissue. It is thought to be due to an excess deposition of extracellular matrix components such as collagen and glycosaminoglycans, usually as a result of injury. Capsular fibrosis is fibrosis that forms a capsule around or encloses an object such as an implant. Capsular fibrosis that occurs with breast implants is referred to a capsular contracture which is a hardening of the tissue surround the implant that causes tissue to tighten around said implant. There are four grades of capsular contraction known as the Baker grades which have been described in the table below.

TABLE 1
Grades of capsular contracture.

| Grade | Palpation |
|---|---|
| Baker I | Breast is soft; implant not palpable |
| Baker II | Breast is solid; implant is palpable and not visible |
| Baker III | Breast is hardened; implant is palpable and visible |
| Baker IV | Breast is hard, deformed, and painful; implant is palpable and |
| | clearly visible |

Foreign body response. Foreign body response (FBR) is a process which takes place whenever any material becomes implanted into the body. The process of implantation injures the tissue around the foreign object, which triggers an inflammatory process. Over a period of weeks to months this inflammatory process develops into a fibrotic response, which envelops and isolates the implanted material. When the foreign material is implanted with the aim of delivering a therapy, both the acute (predominantly inflammatory) and chronic (fibrotic) stages of FBR pose significant challenges to its integrity and therapeutic function. FBR can result in the formation of a foreign body granuloma which consists of protein adsorption, macrophages, multinucleated foreign body giant cells (macrophage fusion/fusogenic macrophages), fibroblasts, and angiogenesis.

Lymphocyte inhibiting activity. Lymphocytes are a type of white blood cell. Lymphocytes include natural killer cells (which function in cell-mediated, cytotoxic innate immunity), T cells (for cell-mediated, cytotoxic adaptive immunity), and B cells (for humoral, antibody-driven adaptive immunity). Lymphocyte inhibiting activity prevents or reduced differentiation or activity of natural killer cells, T cells and B cells.

Myeloid cell inhibiting activity. Myeloid cells are cells that differentiate from progenitor cells giving rise to monocytes, macrophages, dendritic cells, neutrophils, and granulocytes. Myeloid cells are key components of the innate immune response. Myeloid cells also play a key role in linking innate and adaptive immunity, primarily through antigen presentation and recruitment of adaptive immune cells. Myeloid cell inhibiting activity prevents or reduced differentiation or activity of monocytes, macrophages, dendritic cells and granulocytes.

Macrophage inhibiting activity. Macrophages are a heterogeneous population of cells in the body. Derived from monocytes, a type of white blood cell, they undergo differentiation in response to specific signals and act to maintain and restore homeostasis in an individual. Macrophage inhibiting activity prevents or reduces the differentiation or activity of macrophages.

Fusogenic Macrophage inhibiting activity. Fusogenic macrophages (e.g. Arg1⁺ macrophages) are macrophages capable of fusing with other macrophages which can lead to the formation of foreign body giant cells (FBGC's). Fusogenic macrophages and FBGCs are known to release degradative enzymes, ROS, and pro-fibrotic factors, which regulate the recruitment, growth and proliferation of fibroblasts. Macrophage inhibiting activity prevents or reduces the differentiation or activity of fusogenic macrophages or FBCGs.

Fibroblast inhibiting activity. Fibroblasts are a type of cell found in connective tissue that produce the extracellular matrix (ECM) and collagen. They are also the central mediators of the pathological fibrotic accumulation of ECM and the cellular proliferation and differentiation that occurs in response to prolonged tissue injury and chronic inflammation. Fibroblast inhibiting activity prevents or reduces the differentiation or activity of fibroblasts.

Breast Implant Associated Anaplastic Large Cell Lymphoma (BIA-ALCL). BIA-ALCL is a type of lymphoma that can develop around breast implants. BIA-ALCL occurs most frequently in patients who have breast implants with textured surfaces. This is a cancer of the immune system, not a type of breast cancer. BIA-ALCL can develop in the scar tissue capsule and fluid surrounding a breast implant. In some cases, it can spread throughout the body. The current lifetime risk of BIA-ALCL is estimated to be 1:2, 207-1:86,029 for women with textured implants based upon current confirmed cases and textured implant sales data over the past two decades.

All publications mentioned herein are incorporated herein by reference to disclose and describe the methods and/or materials in connection with which the publications are cited.

### Devices and Methods

Described herein are apparatuses (systems and devices including compositions) and methods of making and using them for preventing, inhibiting, or treating implant associated complications and preventing, inhibiting, or treating excess or unwanted fibrous tissue in an individual. Preventing, inhibiting, or treating implant associated complications can include preventing, inhibiting or treating foreign body response (FBR), fibrosis, capsular fibrosis, inflammation at or near the site of implantation, or cancer (e.g. breast implant associated anaplastic large cell lymphoma; BIA-ALCL). In particular described herein are apparatuses (systems and devices) and compositions containing inhibitors of mechanotransduction, as well as methods of making and using them. Molecular targets that can inhibited to impair mechanotransduction can include Rac Family Small GTPase 1 (RAC 1), RAC2, C—C Motif Chemokine Ligand 4 (CCL4), Growth and Arrest and DNA Damage Inducible Alpha (GADD45A), or IQ Motif Containing GTPase Activating Protein 1 (IQGAP1). These apparatuses and compositions can be particularly useful for preventing, inhibiting, or treating (reducing) excess fibrous connective tissue such as that caused by injury or implantation of a medical device into a tissue of an individual.

One aspect of the invention describes a biomedical device including an effective amount of a composition containing a mechanotransduction inhibitor for preventing, inhibiting or treating implant associated complications such as FBR, fibrosis, implant inflammation, BIA-ALCL and in particular for preventing, inhibiting or treating capsular fibrosis. A mechanotransduction inhibitor as described for use herein can be an inhibitor of RAC1, RAC2, CCL4, GADD45A, or IQGAP1.

Inhibitors of RAC1 that find use in the present disclosure include, without limitation, NSC23766, ZINC69391, IA-166, CAS 1177865-17-6 Calbiochem, etc. Inhibitors of RAC2 that find use in the present disclosure include, without limitation, NSC23766, EHT 1684, etc. Inhibitors of CCL4 that find use in the present disclosure include, without limitation, Maraviroc, etc. Inhibitors of IQGAP1 that find use in the present disclosure can include an IQGAP1 WW peptide such as those described in Jameson et al Nat Med. 2013 May; 19(5):626-630 which is hereby specifically incorporated by reference.

Fibrosis can be prevented, inhibited, or treated with a mechanotransduction inhibitor implanted in, inserted in, or placed on the body of an individual in need thereof, such as an individual having or at risk of having fibrosis. A biomedical device as described herein for preventing, inhibiting, or treating fibrosis can be configured for implanting into a body of an individual, for inserting into a body of an individual, or for placing on a body of an individual, the individual having or at risk of having fibrosis. A biomedical device can be configured to replace or repair damaged tissue structures or to prevent or reduce the risk of damaging tissue structures.

In some embodiments, a biomedical device as described herein for administering a mechanotranduction inhibitor can be an implantable biomedical device (e.g., may be an implant) and can be configured (sized and shaped) for implanting into an individual, such as being configured for implanting intraarterially, intraarticularly, intramuscularly, intraocularally, intraperitoneally, intravenously, or subcutaneously. A biomedical device as described herein for administering a mechanotransduction inhibitor can be an implantable biomedical device (e.g., can be an implant) configured to administer the mechanotransduction inhibitor intraarterially, intraarticularly, intramuscularly, intraocularally, intraperitoneally, intravenously, or subcutaneously. In some embodiments, the biomedical device is a breast implant, a catheter, a coil, a heart valve, a lens, a prosthesis, or a stent. A biomedical device can serve as a depot for one or more drugs, which can include a mechanotransduction inhibitor (e.g. an inhibitor of RAC1, RAC2, CCL4, GADD45A, or IQGAP1) and can include one or more other drugs.

In some embodiments, a biomedical device as described herein for administering a mechanotransduction inhibitor can be an insertable biomedical device (e.g., be an insert) and can be configured (sized and shaped) for inserting into an individual, such as into or through a naturally occurring orifice, such as into or through an anus/colon/rectum, an ear (ear canal), an esophagus, a mouth, a nose (a nares), or a vagina. In general such an insertable biomedical device is expellable or readily removable from the body of the individual (and especially without requiring surgery to do so) and can be configured to be expellable or removable from the body of the individual after 1 day, after 2 days, after 3 days, or after a week. In some embodiments, an insertable (or any other) biomedical device as described herein can be biodegradable. A biomedical device as described herein can be an insertable biomedical device and can be configured to administer a composition such as a mechanotransduction inhibitor buccally, intranasally, orally, intrarectally, intravaginally, or sublingually.

In other embodiments, a device can be configured (sized and shaped) for placing on a body of an individual, such as on the skin of the individual. In some embodiments, a device can be a gas, a gel, a liquid, or a solid. A device configured to administer a composition such as a mechanotransduction inhibitor for placing on a body of an individual can be a composition, a dressing (including a wound dressing), or a patch. A device for placing on a body of an individual can be configured for topical delivery or transdermal delivery. In some embodiments, a device configured to administer a composition as described herein can be configured for assisted delivery of the inhibitor, such as by iontophoresis or microinjection (such as with one or a plurality of microneedles). A biomedical device as described herein can be configured for administering a composition ionotophoretically, topically, or transdermally.

In some embodiments, a medical device having a mechanotransduction inhibitor is configured to stay in the body at least 2 weeks, at least 4 weeks, at least 8 weeks, at least 3 months, at least 6 months, or at least one year.

In any of the embodiments described herein, the mechanotransduction inhibitor composition can be coated on an outer surface of a biomedical device or the mechanotransduction inhibitor composition can be impregnated in the biomedical device, or can be both coated on an outer surface and impregnated in the device. A coating can be, for example, from 0.1 um thick (or less) to 10 um thick. In any of the embodiments described herein, the mechanotransduction inhibitor composition can be covalently attached (e.g., through a covalent bond) to the medical device or can be non-covalently attached (e.g., through non-covalent interactions such as van der Waals forces or hydrogen bonding) or can be both covalently attached and non-covalently to the medical device. A mechanotransduction inhibitor can act on the body of an individual while attached to a device or can act after detaching (being detached from a device). A biomedical device as described herein for administering a mechanotransduction inhibitor can be configured to release inhibitor from the biomedical device (such as into a bloodstream or colon) or can be configured to hold inhibitor and not release it or can be configured to do both. Thus a mechanotransduction inhibitor can be delivered locally (e.g., effectively delivered at or close to the implant delivery site) and/or can be delivered beyond the local site, such as delivered systemically.

For example, a biomedical device with covalently attached mechanotransduction inhibitor can be configured to not release the mechanotransduction inhibitor and the composition on the device can be configured to act locally. In other examples, a biomedical device with non-covalently attached mechanotransduction inhibitor can release or be configured to release its payload of mechanotransduction inhibitor and an effective amount of a mechanotransduction inhibitor can only travel a short distance, such as less than 2, less than 5, less than 10 or less than 20 times the longest dimension of the implant (e.g., if the biomedical device is an implant that is 5 mm long in the longest dimension, the device and mechanotransduction inhibitor attached thereto can be configured such that an effective amount mechanotransduction inhibitor only travels 10 mm (e.g., 2 times the longest dimension of the implant) away from the implant.

In other examples, a biomedical device with non-covalently attached mechanotransduction inhibitor can be configured to release its payload of mechanotransduction inhibitor and released mechanotransduction inhibitor can travel a significant distance through the body of an individual, such as through a bloodstream or skin. In some embodiments, a biomedical device can be configured to administer a mechanotransduction inhibitor locally. In some particular embodiments, a biomedical device can be an implant for implanting into an individual in need thereof and can be configured to administer a mechanotransduction inhibitor locally. In some embodiments, a biomedical device can be configured to release some or all of its payload of mechanotransduction inhibitor from the device.

In some embodiments a device having a mechanotransduction inhibitor can be configured for releasing (administering) some or all of its mechanotransduction inhibitor payload systemically. For example, a device having a mechanotransduction inhibitor can be configured for placing in a blood vessel and releasing or administering its mechanotransduction inhibitor payload systemically. A mechanotransduction inhibitor (e.g., an inhibitor of RAC1, RAC2, CCL4, GADD45A or IQGAP1) coating on a device can be a uniform coating or a non-uniform coating.

In some embodiments, a coating can be applied to the surface of a biomedical device without the use of an inert polymer. For example, exemplary embodiments of the drug releasing coatings described herein do not require that the application of an inert polymer layer to the surface of the medical device to bind a therapeutic agent (e.g., an inhibitor of RAC1, RAC2, CCL4, GADD45A or IQGAP1) to the implant's surface. A mechanotransduction inhibitor (e.g., an inhibitor of RAC1, RAC2, CCL4, GADD45A or IQGAP1) coating on the surface of a medical device or impregnated into a device can be attached using a binder. A binder is used to impart cohesive qualities to a composition, and thus ensure that a composition remains intact (as desired) after cohesion of the composition to a biomedical device. In any of the embodiments described herein, a coating composition can be composed of at least one therapeutic agent (e.g., an inhibitor of RAC1, RAC2, CCL4, GADD45A or IQGAP1) in a composition dispersed in a modified, biologically active binders. The therapeutic agent in a coating composition or an impregnation composition can be a mechanotransduction inhibitor and can further include another therapeutic agent, such as anti-inflammatory drug, such as dexamethasone to inhibit inflammation. In some embodiments, no additional therapeutic agent is included in a coating composition or impregnation composition. In some particular examples, a therapeutic agent will be applied to the surface of a biomedical device (e.g., an implantable device) via covalent bonding.

In some embodiments, a coating of a mechanotransduction inhibitor (e.g., an inhibitor of RAC1, RAC2, CCL4, GADD45A or IQGAP1) can be a “cap coating”. A “cap coating” can be applied over a therapeutic agent or drug releasing coating. A “cap coating” can act or can be configured to act as a barrier. A “cap coating” can control or can be configured to control release of the therapeutic agent or drug releasing coating (and for bioactive binders) from a surface of a biomedical device. A cap coating can include a mechanotransduction inhibitor and can include polymers (e.g., silicone-based polymers) having a mechanotransduction inhibitor. A cap coating can have elastomeric properties. In some embodiments, a cap coating, such as one with elastomeric properties, can allow or can be configured to allow the cap coating to be applied to an expandable or flexible medical devices. Accordingly, the elastic properties of the cap coating can permit the coating to be expanded and flexed without comprising the integrity of the cap coating and thereby allowing for the controlled release of therapeutic agents (and biologically active binders) from the surface of the implant. Accordingly, the controllable release of these components at the site of implantation can treat, reduce or prevent pathologies associated with the implantation of the device.

Additionally, the therapeutic agents (and biologically active binders) can be controllably released from the surface of the medical implant by providing a cap coating on the biomedical device, and especially on a biomedical implant device. A biomedical device can have a mechanotransduction inhibitor covalently bonded thereto. A mechanotransduction inhibitor, can be covalently bonded by physically blending or dispersing it with a polymers such as inert polymer. Such an inert polymers may not possess any known pharmacological activity and in some cases may only serve as a carrier or binder for the mechanotransduction inhibitor. The use of inert polymers for coating may allow larger doses of drugs to be applied to the medical device surface and concomitantly larger amounts of the drug may be released and especially with less toxicity.

A biomedical device as described herein is generally biocompatible and is made from a biocompatible material. A biomedical device as described herein can be either biodegradable or may be non-biodegradable or can be a combination in which a first part is biodegradable and a second part is non-biodegradable. The biomedical device can be made from, including being coated or layered with, acrylic, hydroxyethyl methacrylate (HEMA), methacrylate, polyamine, polycaprolactone, polyglycolic acid, polyester, polyether, polypropylene, polysiloxane, polyurethane, polylactic acid, silicone, another biodegradable or non-biodegradable co-polymer or polymer, a metallic material, a non-metallic material, or combinations thereof. In a particular example, the biomedical device is made from a biocompatible silicone. In some examples, a biomedical device can contain many particles, such as a plurality of nanoparticles containing a mechanotransduction inhibitor.

Another aspect of the invention provides a method of preventing, inhibiting, or treating fibrosis (e.g., capsular fibrosis) in an individual in need thereof. Any of these methods may include selecting an individual that has or is at risk of having fibrosis (e.g., capsular fibrosis). An individual that has fibrosis (e.g., capsular fibrosis) may, for example, have fibrosis encapsulating a previously placed biomedical device (implant) as a result of receiving the device implant). An individual at risk of having fibrosis may be an individual who is having or planning to have a biomedical device (implant) placed in the body of the individual and there may be a risk that fibrosis and especially capsular fibrosis occurs around the implant. Any of these methods may include administering to the individual a composition having an effective amount of a mechanotransduction inhibitor and may include thereby preventing, inhibiting, or treating capsular fibrosis in the individual. Any of these methods may include administering to the individual a composition having an effective amount of a mechanotransduction inhibitor and may include thereby preventing, inhibiting, or treating capsular fibrosis in the individual. Any of these methods may include administering to the individual a composition having an effective amount of macrophage inhibiting activity and may include thereby preventing, inhibiting, or treating capsular fibrosis in the individual. Any of these methods may include administering to the individual a composition having an effective amount of a fusogenic macrophage inhibiting activity and may include thereby preventing, inhibiting, or treating capsular fibrosis in the individual. Any of these methods may include administering to the individual a composition having an effective amount of a Arg1⁺ macrophage inhibiting activity and may include thereby preventing, inhibiting, or treating capsular fibrosis in the individual. Any of these methods may include administering to the individual a composition having an effective amount of a lymphocyte inhibiting activity and may include thereby preventing, inhibiting, or treating capsular fibrosis in the individual. Any of these methods may include administering to the individual a composition having an effective amount of a fibroblast inhibiting activity and may include thereby preventing, inhibiting, or treating capsular fibrosis in the individual. Any of these methods may include administering to the individual a composition having an effective amount of a foreign body giant cell inhibiting activity and may include thereby preventing, inhibiting, or treating capsular fibrosis in the individual. In addition to preventing, inhibiting, or treating capsular fibrosis, the above methods may also be utilized for preventing, inhibiting, or treating implant inflammation or implant associated cancers (e.g. BIA-ALCL).

In some embodiments, the amount of a mechanotransduction inhibitor administered can be titrated to effectively allow no or little fibrosis. In some embodiments, the amount of a mechanotransduction inhibitor administered can be titrated to effectively allow some fibrosis. For example, fibrosis can be a means for anchoring and holding a biomedical device (implant) so that it remains in or close to an implant location.

Any of these methods can include wherein the composition (a mechanotransduction inhibitor) is administered intraarterially, intraarticularly, intramuscularly, intraocularally, intraperitoneally, intravenously, or subcutaneously. Any of these methods can include wherein the composition is administered buccally, intranasally, orally, intrarectally, intravaginally, or sublingually. Any of these methods can include wherein the composition is administered iontophoretically, topically, or transdermally. Any of these methods can include wherein the composition is administered systemically. For example, a composition can be non-covalently attached to a biomedical device and released from the biomedical device, such as into skin or a bloodstream of the individual. Any of these methods can include wherein the composition is administered locally. For example, a composition can be covalently attached to a biomedical device (an implant). In any of these or other biomedical devices the composition can remain attached or can be released locally, such that it is configured and able to act at or near the site of implantation of the implant. Such attached or locally released composition can advantageously have a relatively high concentration of the composition for preventing, reducing, or treating fibrosis while preventing or reducing unwanted effects further away from the site of implantation. As indicated elsewhere herein, mechanotransduction inhibition can have pleiotropic effects in the body and in some examples it can be desirable to maintain the composition locally.

In any of the methods described herein wherein the biomedical device comprising the composition is an implant, the method may further include the step of making an incision in the individual and implanting the implant into the individual through the incision. In any of these methods or devices described herein, the implant may be further configured to treat a medical issue or cosmetic issue other than fibrosis, such as a breast issue, a dental issue an ear or hearing issue, an eye or sight issue, a heart issue, a joint issue, a skin issue, a spinal issue, or a uterine issue. In some embodiments, an issue as addressed by the methods or devices herein may be due to reduced or loss of function (such as a damaged spinal disc).

In some embodiments, an issue as addressed by the methods or devices herein may be due to excess or unwanted tissue or tissue function (such as a tumor). In some embodiments, an issue as addressed by the methods or devices herein may be an enhancement or an enlargement, such as a breast enhancement or breast enlargement. An implant configured to administer or deliver a mechanotransduction inhibitor may be an artificial eye lens, a breast implant, a cardioverter defibrillator, a cochlear implant, a dental implant, an ear tube, an intra-uterine device, a metal implant, a pacemaker, a silicone implant, a spinal implant (e.g., an artificial disc, rod, screw), a surgical mesh, a shunt, or a stent. Any of these implants may be coated with and/or impregnated with a mechanotransduction inhibitor including any mechanotransduction inhibitor (e.g., an inhibitor of RAC1, RAC2, CCL4, GADD45A, or IQGAP1) as indicated elsewhere herein.

In any of the methods described herein wherein the biomedical device including the composition is an insert, the method may further include inserting the insert into an orifice of the individual as indicated elsewhere herein. In any of the methods described herein wherein the biomedical device including the composition is or includes a skin interface, the method may further include placing the skin interface on a skin of the individual. In any of the methods or devices described herein the composition prevents, inhibits, or reduces macrophage activity or differentiation or is configured to prevent, inhibit, or reduce macrophage activity or differentiation by at least 10%, at least 20%, at least 30%, at least 40% or at least 50%, such as when compared with a method or device not having the mechanotransduction inhibitor. In any of the methods or devices described herein the composition prevents, inhibits, or reduces Arg1⁺ macrophage activity or differentiation or is configured to prevent, inhibit, or reduce Arg1⁺ macrophage activity or differentiation by at least 10%, at least 20%, at least 30%, at least 40% or at least 50% such as when compared with a method or device not having the mechanotransduction inhibitor. In any of the methods or devices described herein the composition prevents, inhibits, or reduces lymphocyte activity or differentiation or is configured to prevent, inhibit, or reduce lymphocyte activity or differentiation by at least 10%, at least 20%, at least 30%, at least 40% or at least 50% such as when compared with a method or device not having the mechanotransduction inhibitor. In any of the methods or devices described herein the composition prevents, inhibits, or reduces fibroblast activity or differentiation or is configured to prevent, inhibit, or reduce fibroblast activity or differentiation by at least 10%, at least 20%, at least 30%, at least 40% or at least 50% such as when compared with a method or device not having the mechanotransduction inhibitor. In any of the methods or devices described herein the composition prevents, inhibits, or reduces fusogenic macrophage activity or differentiation or is configured to prevent, inhibit, or reduce fusogenic macrophage activity or differentiation by at least 10%, at least 20%, at least 30%, at least 40% or at least 50% such as when compared with a method or device not having the mechanotransduction inhibitor. In any of the methods or devices described herein the composition prevents, inhibits, or reduces foreign body giant cell activity or differentiation or is configured to prevent, inhibit, or reduce foreign body giant cell activity or differentiation by at least 10%, at least 20%, at least 30%, at least 40% or at least 50% such as when compared with a method or device not having the mechanotransduction inhibitor.

In any of the methods or devices described herein the composition prevents, inhibits, or treats or is configured to prevent, inhibit, or treat collagen deposition e.g., by at least 10%, at least 20%, at least 30%, at least 40% or at least 50% such as when compared with a method or device not having the mechanotransduction inhibitor (e.g. an inhibitor of RAC1, RAC2, CCL4, GADD45A, or IQGAP1). In any of the methods or devices described herein the composition prevents, inhibits or treats a foreign body response or is configured to prevent, inhibit or treat or e.g., by at least 10%, at least 20%, at least 30%, at least 40% or at least 50% such as when compared with a method or device not having the mechanotransduction inhibitor (e.g., an inhibitor of RAC1, RAC2, CCL4, GADD45A, or IQGAP1).

In any of the methods or devices described herein the individual may be an animal, such as a human, a domestic or farm animal such as a cat, cow, dog or pig, or a wild animal.

Another aspect of the invention provides a composition comprising an effective amount of a mechanotransduction inhibitor. An effective amount of a mechanotransduction inhibitor may be an amount effective to prevent, reduce or treat fibrosis (capsular fibrosis) and/or to prevent, inhibit, reduce or treat any of the conditions described herein (such as to prevent, inhibit, or reduce macrophage activity or differentiation, prevent, inhibit, or reduce macrophage Arg1⁺ activity or differentiation, prevent, inhibit, or treat collagen deposition, a prevent, inhibit, or treat foreign body response) such as at least 10%, at least 20%, at least 30%, at least 40% or at least 50% or not more than 10%, 20%, 30%, 40%, or 50% than such as when compared with a vehicle not having the mechanotransduction inhibitor (e.g., an inhibitor of RAC1, RAC2, CCL4, GADD45A, or IQGAP1).

Another aspect of the invention provides a composition comprising an effective amount of a mechanotransduction inhibitor and a secondary treatment modality. Secondary treatment modalities that find use in the present disclosure may be any treatment used to alleviate implant associated complications such as foreign body response (FBR), capsular fibrosis, inflammation at or near the site of implantation, cancer (e.g. BIA-ALCL). For instance, the composition may comprise a TGF-β receptor inhibitor, corticosteroids, bleomycin, 5-Flurouracil, prednisone, minocycline, allopurinol, colchicines, or cyclosporine in addition to a mechanotransduction inhibitor. A Wnt1 inhibitor or a monoterpenoid such as 1,8-cineol as described in international application PCT/US2020/042248, which is hereby specifically incorporated by reference.

### Animal Model and Drug Screening

Altering tissue-scale forces around implants using mechanically stimulating implants (MSIs) produces human-like FBR capsule architecture in mice. In animal models provided herein, a high mechanical stress environment is created independent of implant size or chemistry. To accomplish this, an implantable device encapsulating a small motor is used to produce intermittent in situ implant vibration. This increases the mechanical loading of the surrounding tissue. Mechanically stimulating implants (MSIs) in mice can generate higher extrinsic forces from the surrounding host tissue and result in a 100-fold increase in mechanical stress at the implant-tissue interface, e.g. from about 10 kPa, from about 15 kPa, from about 20 kPa, to about 50 kPa, to about 30 kPa, and may be around 24-25 kPa, which is similar to that human implants. This model provides a unique platform to examine the effect of the increased tissue-scale forces alone on the subsequent foreign body response. The implant may be vibrated from about 10 minutes to about 1 hour per day, e.g. about 10, 20, 30, 40, 50, 60 minutes.

In some embodiments, a small animal model, e.g. mouse model, is provided, the animal comprising a mechanically stimulating implant that produces intermittent in situ implant vibration.

When compared to standard implants, these mechanically stimulated implants develop FBR capsules with significantly increased collagen deposition, increased collagen maturity, and a higher level of activation of myofibroblasts. With this model, human-like FBR capsule architecture can be recreated in mice. Both an MSI and control, standard, implants are made of the same material, e.g. silicone, with the same geometry.

An MSI can be fabricated of a standard material, e.g. polydimethylsiloxane (PDMS) of a suitable size for the animal. In a mouse the implant may be, for example, a cylinder of 1-2 cm diameter and 0.5 to 1 cm diameter. A prefabricated coin motor is placed in the elastomer solution before curing. To enable in situ vibration of MSIs, the wires from the implant can be guided through the skin. The implant can be placed, for example, in the dorsum of the mouse.

The animal models find use in determining factors involved in development of FBR, for example cells, proteins, genes, and the like. The animal models also find use in screening compounds for activity in the inhibition of FBR.

Candidate agents of interest are biologically active agents that encompass numerous chemical classes, primarily organic molecules, which may include organometallic molecules, inorganic molecules, genetic sequences, etc. Of interest are inhibitors of proteins determined to be involved in FBR as disclosed herein. An important aspect of the invention is to evaluate candidate drugs with the identified biological response functions. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, anti-inflammatory agents, hormones or hormone antagonists, ion channel modifiers, and neuroactive agents. Exemplary of pharmaceutical agents suitable for this invention are those described in, “The Pharmacological Basis of Therapeutics,” Goodman and Gilman, McGraw-Hill, New York, N.Y., (1996), Ninth edition.

The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1 μl to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for biological activity by treating an animal model as disclosed herein and comparing to negative controls in the absence of treatment for fibrotic responses.

Flow cytometry may be used to quantitate parameters such as the presence of cells of interest using cell surface proteins or conformational or posttranslational modification thereof; intracellular or secreted protein, where permeabilization allows antibody (or probe) access, and the like. Brefeldin A is commonly utilized to prevent secretion of intracellular substances. Flow cytometry methods are known in the art, and described in the following: Flow Cytometry and Cell Storing (Springer Lab Manual), Radbruch, Ed., Springer Verlag, 2000; Ormerod, Flow Cytometry, Springer Verlag, 1999; Flow Cytometry Protocols (Methods in Molecular Biology, No 91), Jaroszeski and Heller, Eds., Humana Press, 1998; Current Protocols in Cytometry, Robinson et al., eds, John Wiley & Sons, New York, N.Y., 2000. The readouts of selected parameters are capable of being read simultaneously, or in sequence during a single analysis, as for example through the use of fluorescent antibodies to cell surface molecules. As an example, these can be tagged with different fluorochromes, fluorescent bead, tags, e.g. quantum dots, etc., allowing analysis of up to 4 or more fluorescent colors simultaneously by flow cytometry. Plug-flow flow cytometry that has the potential to automate the delivery of small samples from unpressurized sources at rates compatible with many screening and assay applications, may allow higher throughput, compatible with high throughput screening, Edwards et al. (1999) Cytometry 37:156-9.

Both single cell multiparameter and multicell multiparameter multiplex assays, where input cell types are identified and parameters are read by quantitative imaging and fluorescence and confocal microscopy are used in the art, see Confocal Microscopy Methods and Protocols (Methods in Molecular Biology Vol. 122.) Paddock, Ed., Humana Press, 1998. These methods are described in U.S. Pat. No. 5,989,833 issued Nov. 23, 1999.

The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel et al., eds, John Wiley & Sons, New York, N.Y., 2000; Freeman et al. (1999) Biotechniques 26(1):112-225; Kawamoto et al. (1999) Genome Res 9(12):1305-12; and Chen et al. (1998) Genomics 51(3):313-24, for examples.

### EXPERIMENTAL

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” to another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of’ or alternatively “consisting essentially of’ the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/− 0.1% of the stated value (or range of values), +/− 1% of the stated value (or range of values), +/− 2% of the stated value (or range of values), +/− 5% of the stated value (or range of values), +/− 10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others.

Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure.

Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

### Examples

### Example 1

A third, independent variable was identified on FBR, i.e., allometric tissue-scale forces that play a central role in foreign body response. It is important to note that FBR responses were compared to standard murine implants and MS's using exactly the same material and with exactly the same implant geometry. By varying only tissue-scale forces, it was possible to dramatically alter the molecular and cellular response to recreate human-like FBR in mice. Thus, it has been shown that extrinsic tissue-scale forces, which increase with body size can drive the biology of FBR, independent of both material chemistry and mechanical properties. These results provide an explanation for the long-standing conundrum in the field regarding the significant inter-species variability in FBR, despite the fact that the molecular machinery responsible for inflammation and fibrosis is highly conserved among species. Larger sized organisms, such as humans, experience higher tissue-scale forces because of allometric scaling that then drive subsequent FBR formation.

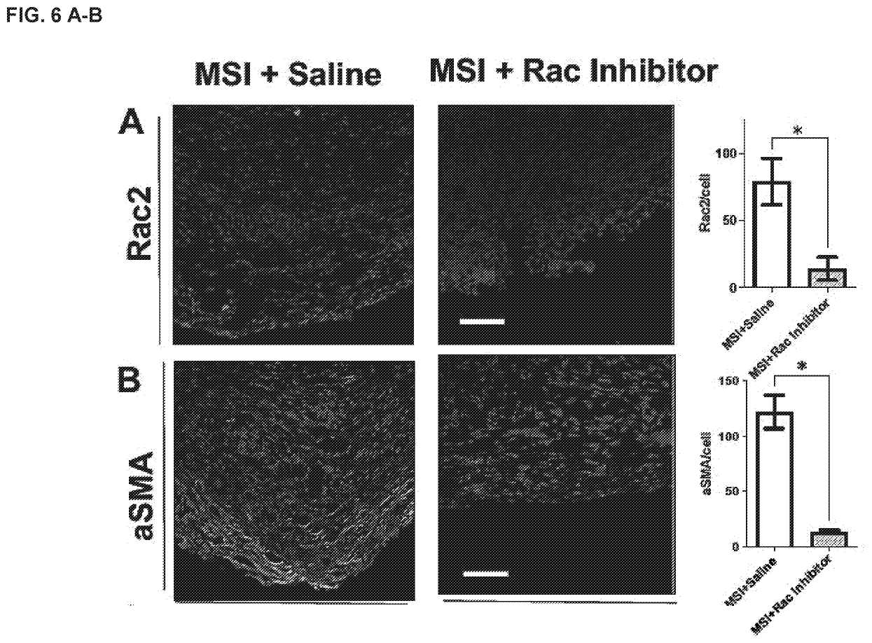

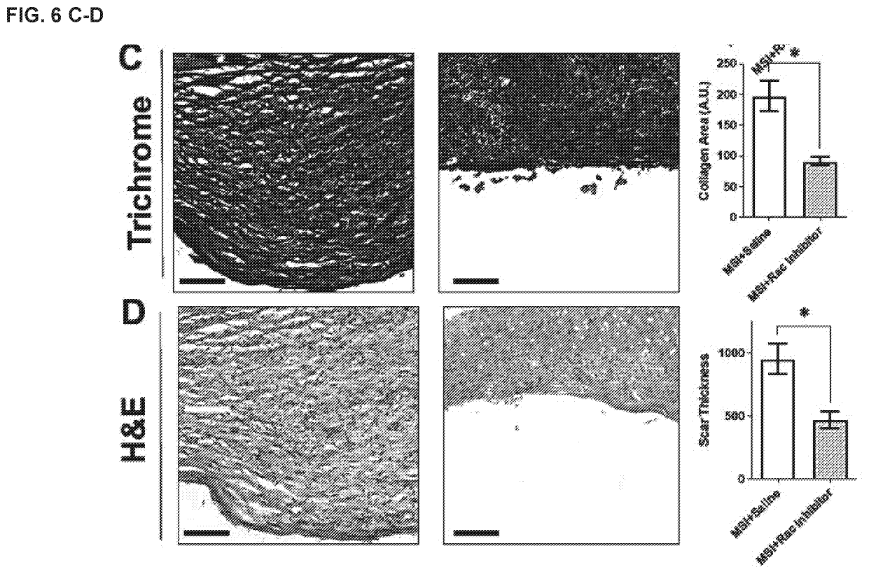

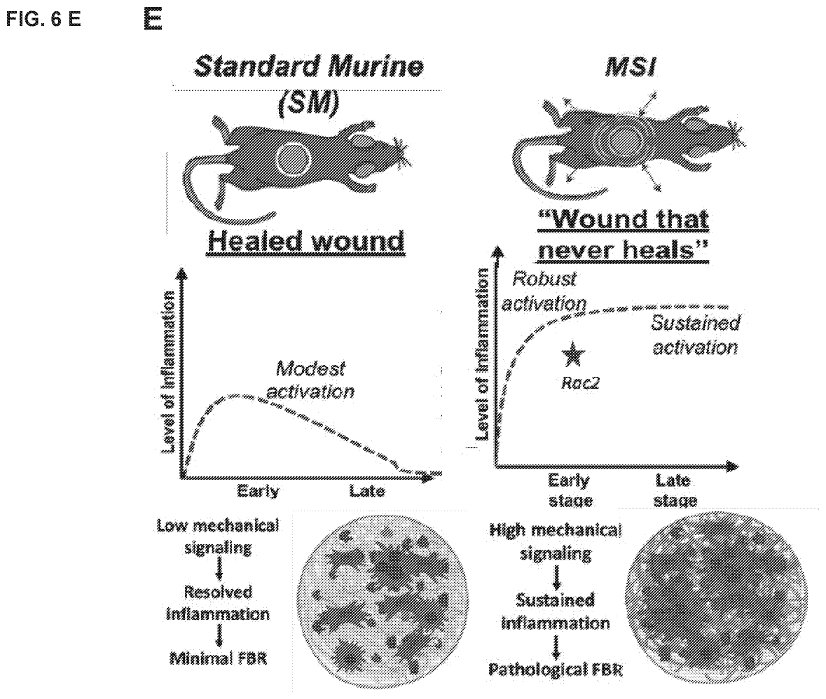

The wound healing cascade in humans is characterized by sequential inflammatory and fibrotic phases and eventual quiescence. FBR begins as a wound healing response to the local tissue damage that occurs during surgical implantation of a device as well, but high levels of extrinsic mechanical forces on the implant (in humans) by the surrounding tissue creates a high mechanical stress environment that leads to a sustained inflammatory response. In standard murine implants with 100-fold lower mechanical stress than in humans, a modest inflammatory response was observed at the early stage, which subsided by later timepoints like a “healed wound” (FIG. 6E). In contrast, both humans and murine MS's generate a 100-fold increased stress environment to perpetuate a sustained presence of mechanically activated immune cells at the implant-tissue interface. Thus, increased tissue-scale forces, shaped by allometric scaling principles, result in a “wound that never heals”, which leads to pathological FBR. In fact, allometric tissue-scale forces may provide the missing link explaining key aspects of pathological FBR including the activation of fusogenic macrophages, MHC class II lymphocytes and myofibroblasts.

Importantly, extrinsic tissue-scale forces were found to result in the mechanical activation of Rac2 signaling in a unique population of immune cells and results in a gene signature that is conserved in both murine and pathologic human tissue capsules. These findings reveal a bi-directed interplay between immune cells and fibroblast activity and suggest that mechanoresponsive immune cells under elevated tissue-scale forces may actually drive and regulate the activation of fibroblasts. The current findings provide a novel mechanistic link for the activation of immune cells in FBR, namely, Rac2 signaling in immune cells activated by allometric tissue-scale forces at the implant-tissue interface.

Since allometric tissue properties in humans cannot be altered because they are inherent to the size of the organism, biomolecular and pharmacologic strategies will be required to create truly biointegrative devices. It was demonstrated that pharmacological inhibition of Rac2 could serve as an effective therapy for humans receiving biomedical implants to prevent FBR, increasing patient quality of life and reducing implant failure rates. Collectively, these findings provide novel insights into FBR and have profound implications for the design and safety of all implantable medical devices in humans.

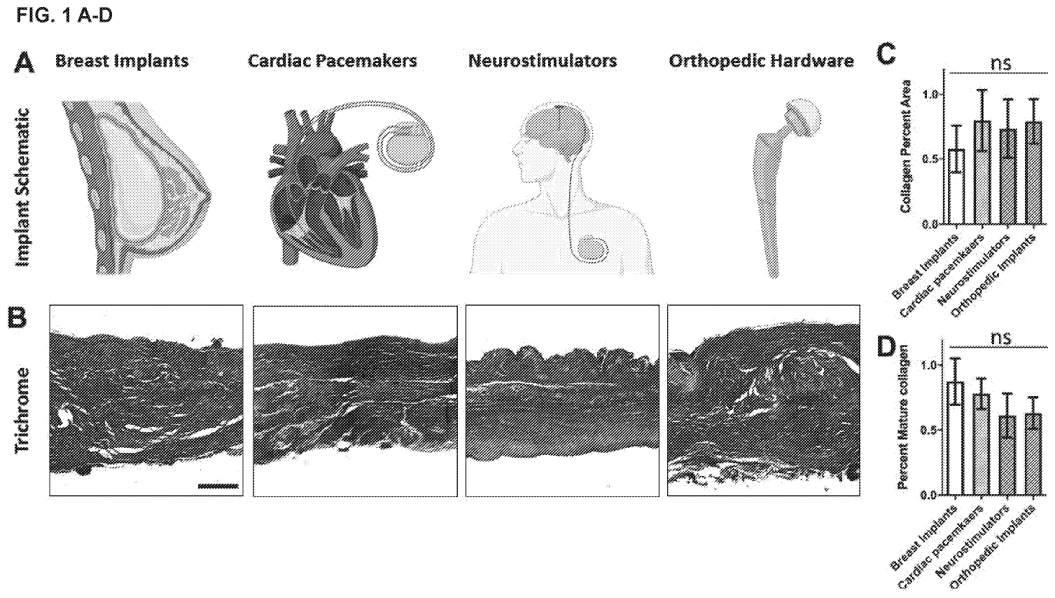

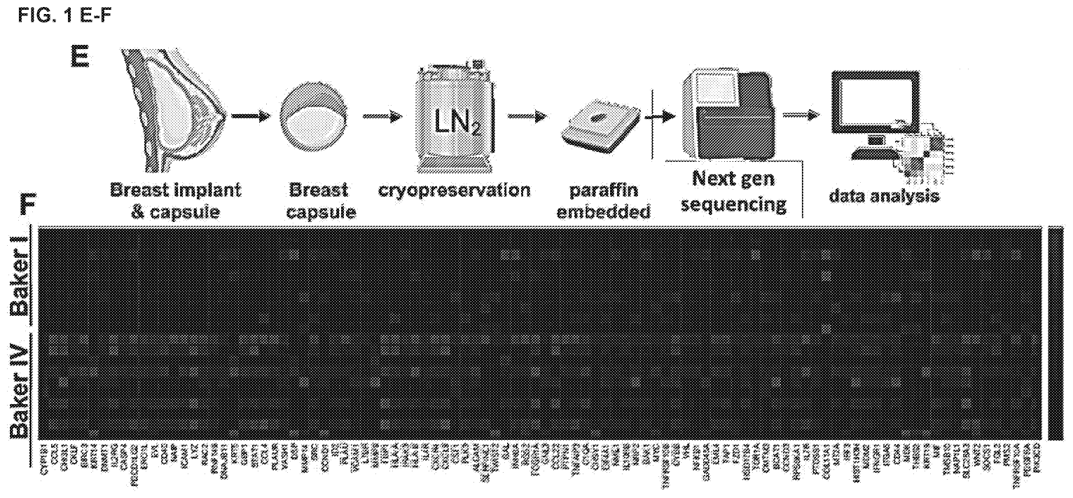

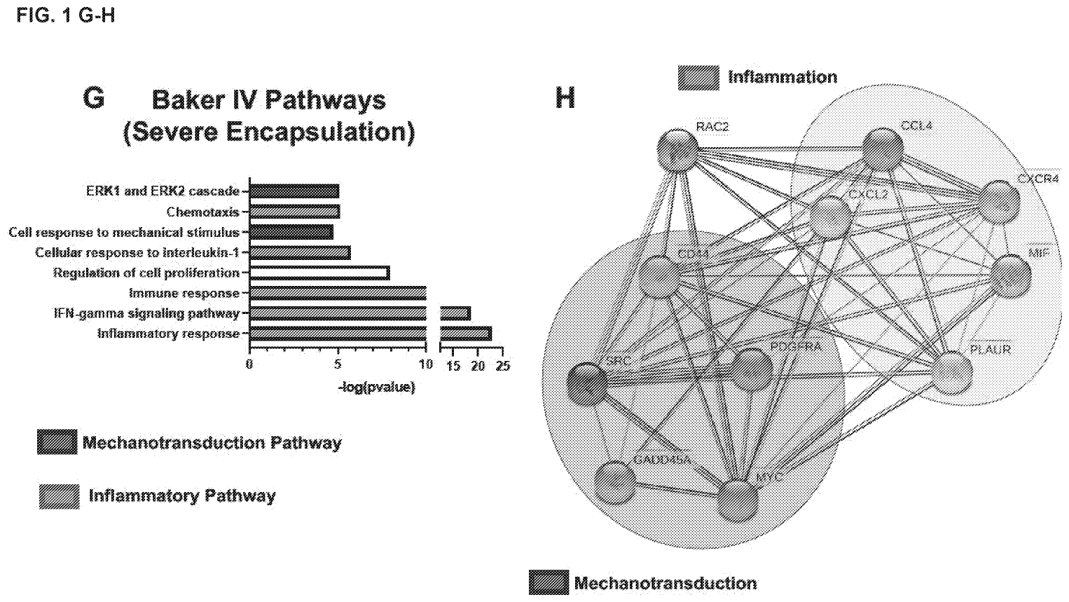

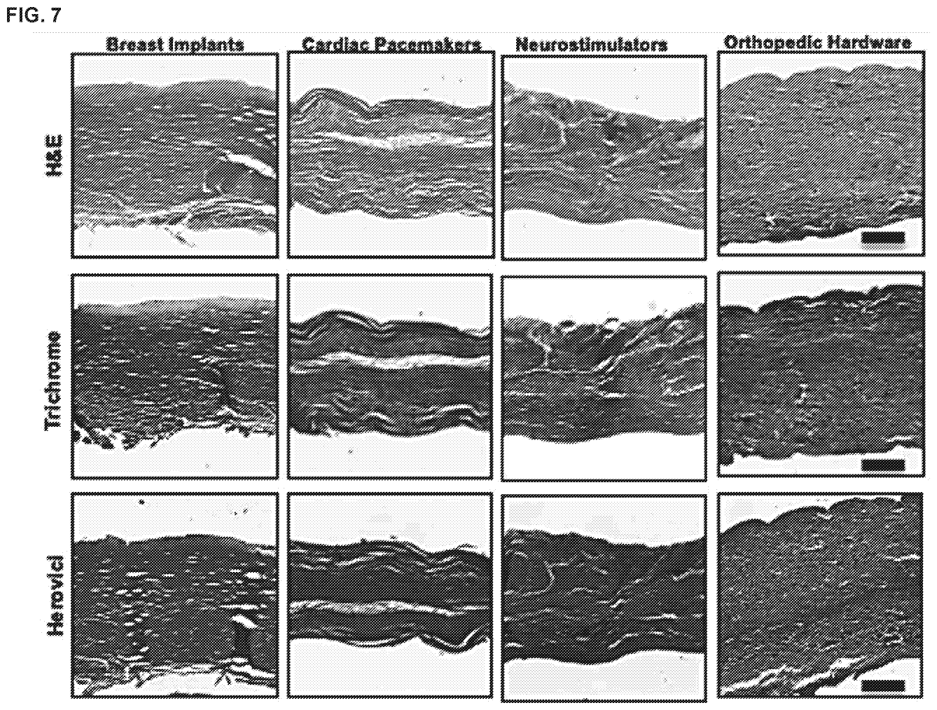

FBR in humans is characterized by similar fibrotic encapsulation across a diverse array of implants, regardless of implant chemistry or mechanical properties. To understand the importance of material properties on human FBR, fibrotic capsules were analyzed from a diverse array of biomedical implants. It was found that the fibrous capsule derived from silicone-based breast implants, titanium-based pacemakers, neurostimulators, and mixed alloybased orthopedic implants were all strikingly similar in tissue architecture (FIG. 1A-B, FIG. 7). All FBR capsules analyzed were predominantly composed of mature type I collagen with organized and aligned fibers, characteristic of scar tissue under relatively high mechanical forces (FIG. 1C, D, FIG. 7). This is the first reported comparison of human FBR across diverse implant types with histological analyses. Because these implants were all made of different materials, it was postulated that implant chemistry and mechanical properties were not sufficient to explain the mechanisms that underlie FBR.

Pathological FBR in humans is characterized by increased mechanotransduction and inflammatory signaling. To explore other, previously unidentified variables that may be involved in FBR, implant capsules were analyzed from identical biomedical implants that exhibited different degrees of severity of foreign body response (FBR) in human patients. Fibrosis around breast implants is conventionally classified using the Baker system, where Baker I is the least severe and represents cases with minimal clinically observable implant capsule contracture, while Baker IV represents the most severe cases that display a sustained inflammatory reaction, pronounced fibrotic contracture, and pain. mRNA isolated from human tissue specimens of both mild and severe FBR observed in humans around implants made of the exact same silicone material using a next generation sequencing-based quantitative assay against a biomarker Panel (HTG Molecular), which consisted of 2500+ known biomarkers for inflammation and fibrosis were analyzed (FIG. 1E, F).

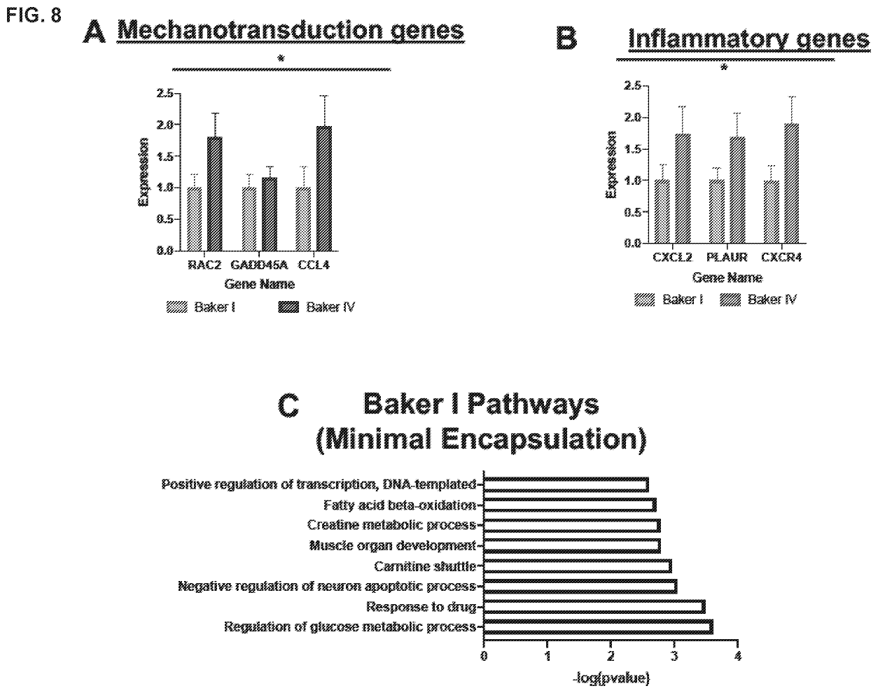

The top genes that were upregulated in severe Baker IV breast implant capsules were identified and characterized using the Database for Annotation, Visualization, and Integrated Discovery (DAVID) pathway analysis (FIG. 1G). Genes upregulated were observed in Baker IV implants (at high risk for FBR-mediated implant failure) were critically involved in mechanical signaling pathways including “Positive regulation of Erk1 and Erk-2 cascade” and “Cellular response to mechanical stimulus” (FIG. 1G). Baker IV implant capsules also showed upregulation of inflammatory signaling related to chemotaxis, cellular response to interleukin-1, and immune response pathways (FIG. 1G). In contrast, the more mild Baker I implants only displayed modest upregulation of pathways related to glucose and fat metabolism (FIG. 8).

Identification of RAC2 mediated mechanotransduction and inflammation in human pathological FBR. The top Baker IV markers were screened to identify genes that may be involved in the mechanical activation of inflammation. Most interestingly, RAC2 was found to be, a hematopoietic-specific Rho-GTPase inflammatory mechanotransduction marker, significantly upregulated in the Baker IV implant specimens (FIG. 8). RAC2 is a signal transduction molecule, which mediates the recruitment and activation of immune cells and has been shown to be activated by mechanical forces. The potential role of RAC2 was further confirmed in human pathological FBR using STRING (Search Tool for the Retrieval of Interacting Genes/Proteins), a pathway analysis tool that predicts gene-gene interactions. Strikingly, RAC2 was a central gene found to interact with the mechanotransduction signaling and inflammatory signaling genes upregulated in Baker IV specimens (FIG. 1H). Since both Baker I and Baker IV implants are made of the same material (silicone), these results strongly suggested that pathological FBR in humans is mediated by RAC2 mechanotransduction signaling, independent of implant chemistry or implant mechanical properties. Thus, it was hypothesized that mechanical forces may mediate immune-cell specific mechanotransduction to generate pathological FBR and sought to test this hypothesis in a novel murine model.

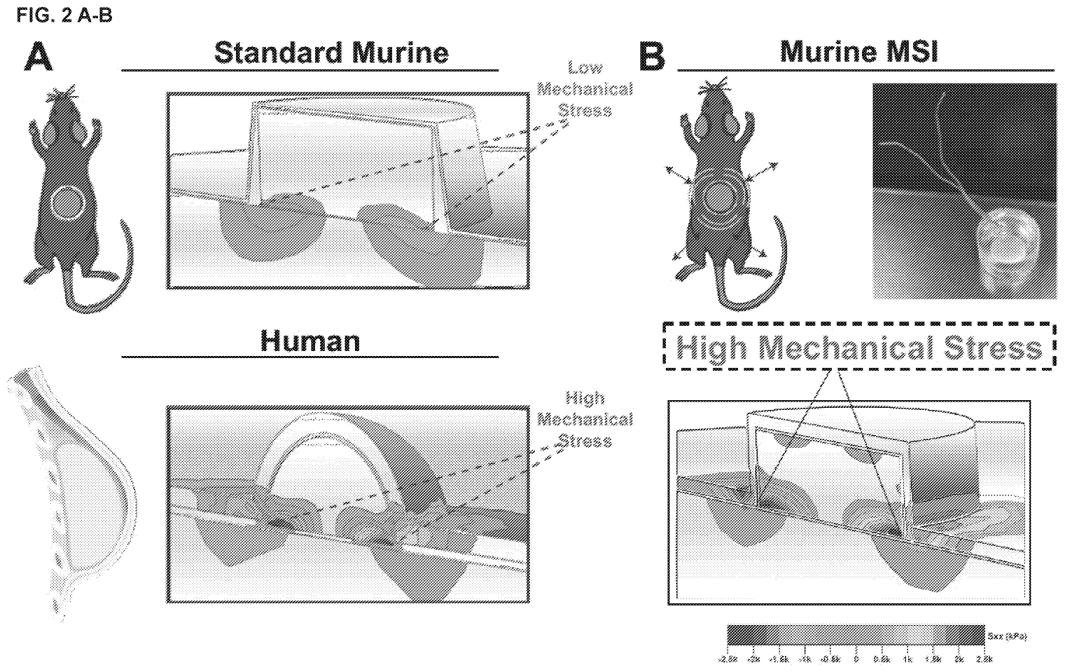

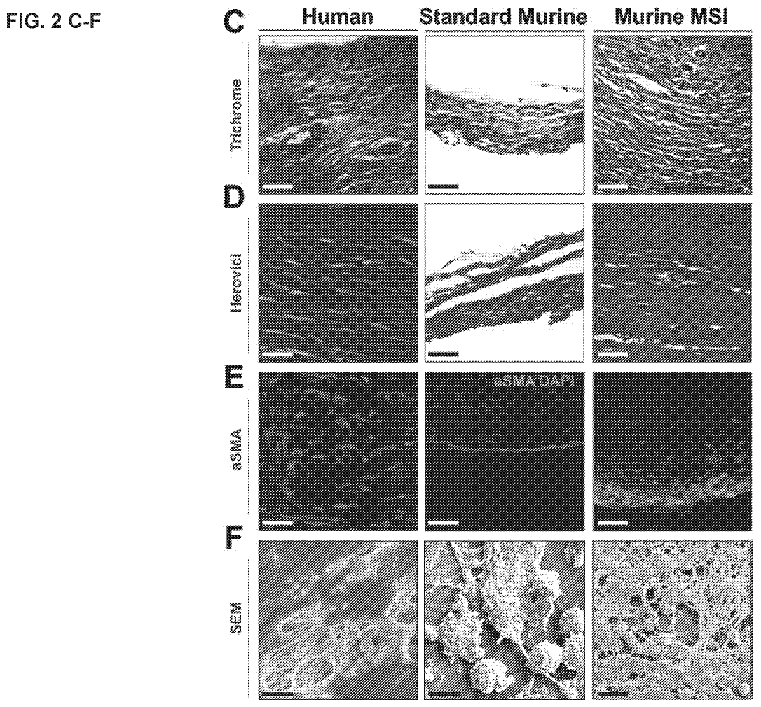

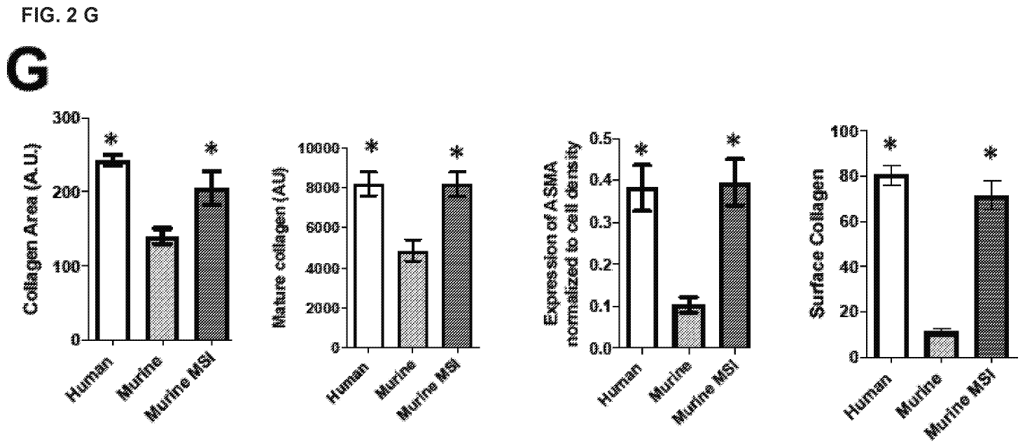

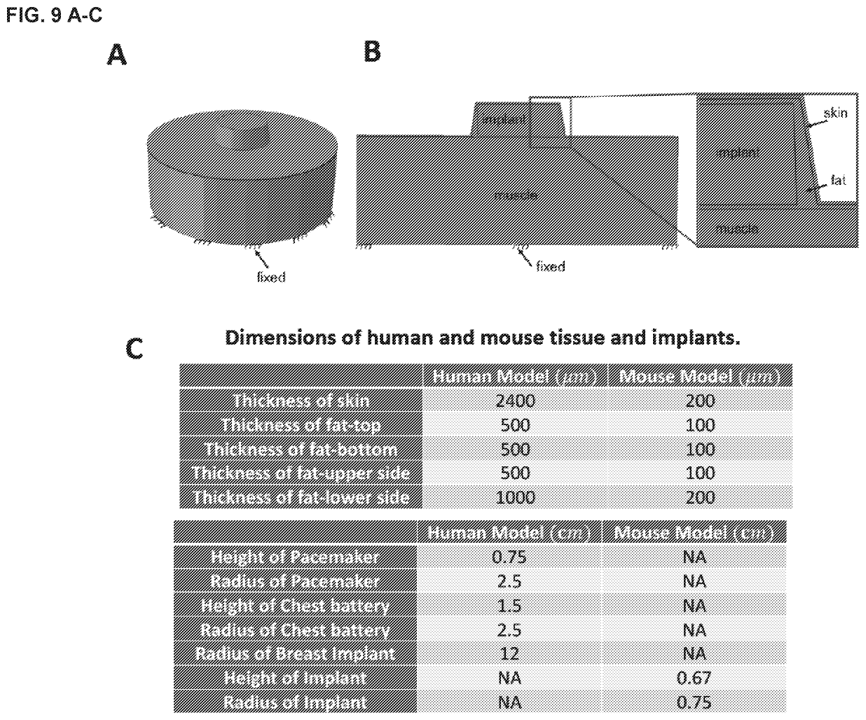

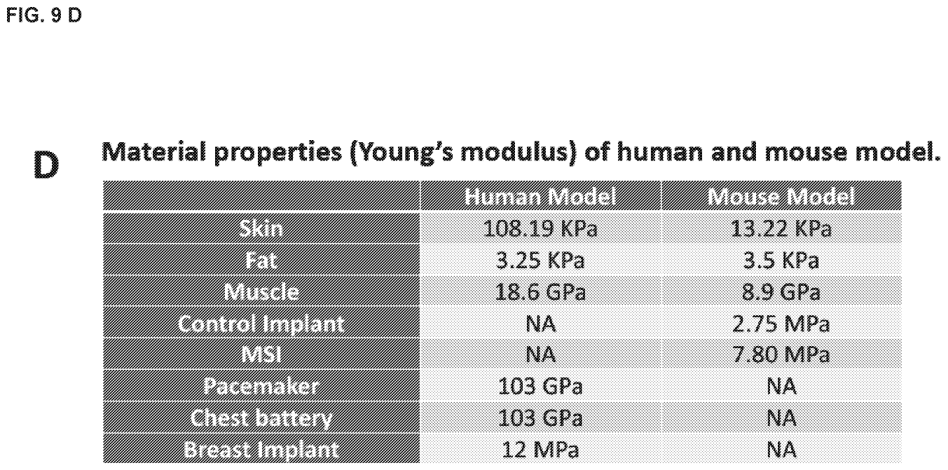

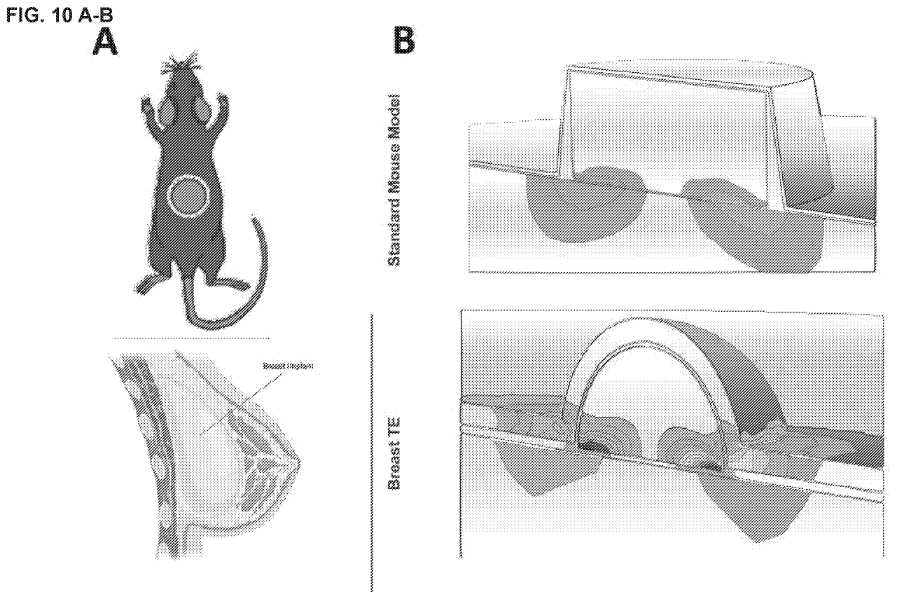

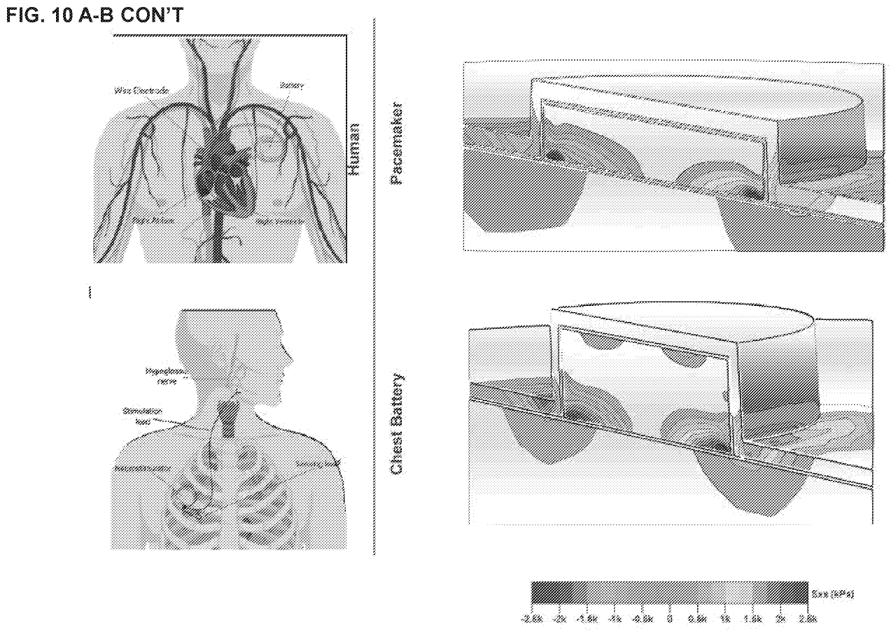

Standard murine models are unable to recreate the high mechanical stress environment found around implants in humans due to allometric scaling of tissue properties. Mechanotransduction signaling is thought to be impacted primarily by the material properties of the implants itself. However, extrinsic forces from the surrounding host tissue on the implant also result in mechanical loading of the implant, and the sum of these two components can be quantified as the local mechanical stress at the implant-tissue interface. To investigate how these extrinsic tissue-scale forces play a role in the development of FBR, the local mechanical stress patterns were first modeled at the implant-tissue interface in mice and humans using finite element modeling (FEM) (ABAQUS, version 2017, SIMULIA, Providence, RI). The predicted maximal stress around a standard silicone murine implant was 0.2 kPa, whereas in humans this was over 20 KPa (FIG. 2A, FIG. 10). Further, we determined that the contribution of extrinsic tissue properties on the ultimate mechanical stress was significantly greater than that of implant properties themselves. Variations in implant geometry and implant material stiffness also resulted in minimal changes to the predicted mechanical stress at the implant-tissue interface in this model. This resulted in a similar predicted maximal stress (20-23 kPa) around both silicone-based breast implants and much stiffer titanium-based implants such as pacemakers and neurostimulators in humans (FIG. 10), which explains the similar fibrotic encapsulation observed around the different types of human implants (FIG. 1A-D). Thus, owing to the large allometric differences between humans and mice in both tissue size and tissue mechanical properties, the effective mechanical stress around biomedical implants in humans is about 100-fold higher than that around implants in standard preclinical models such as mice (FIG. 2A, FIG. 9,10).

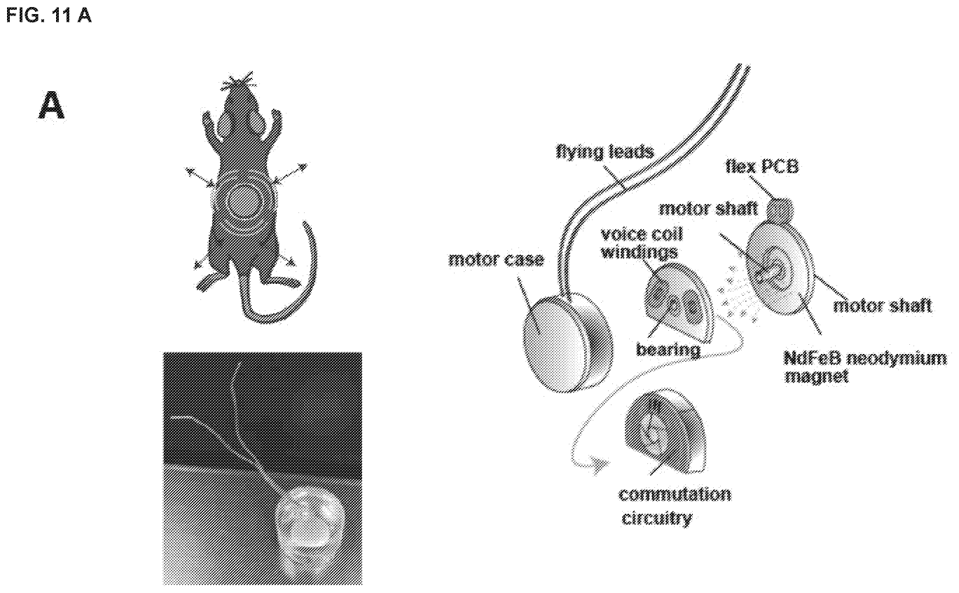

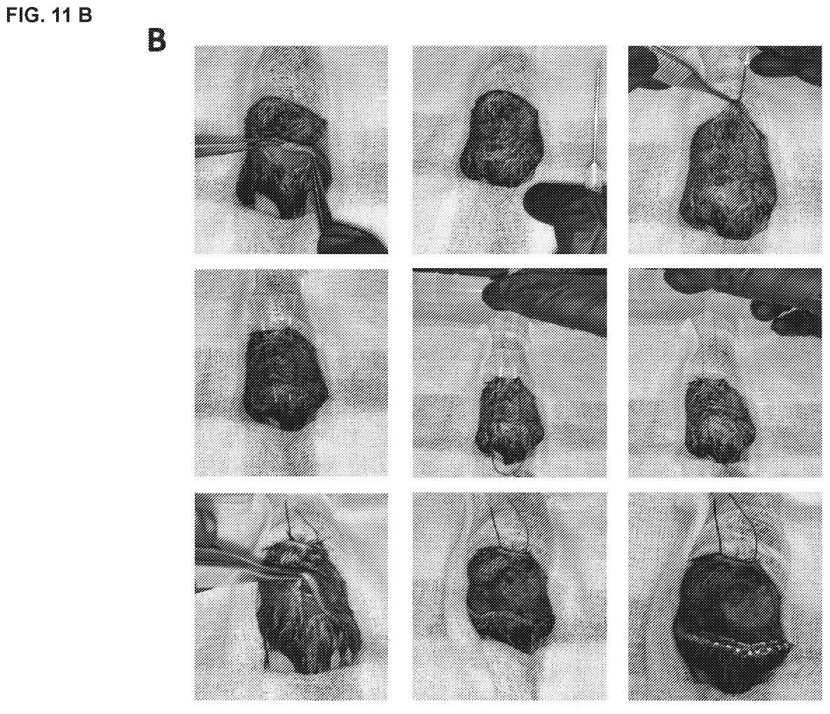

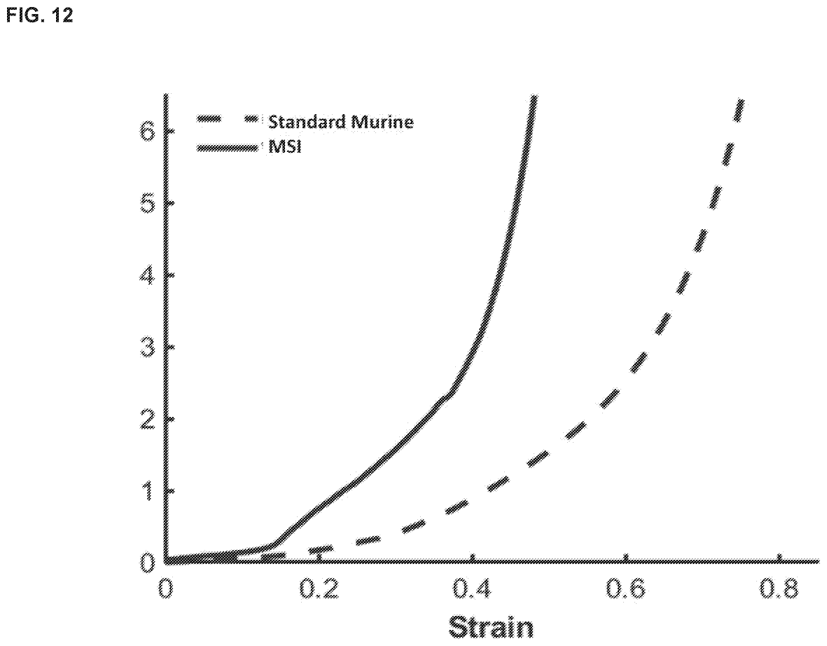

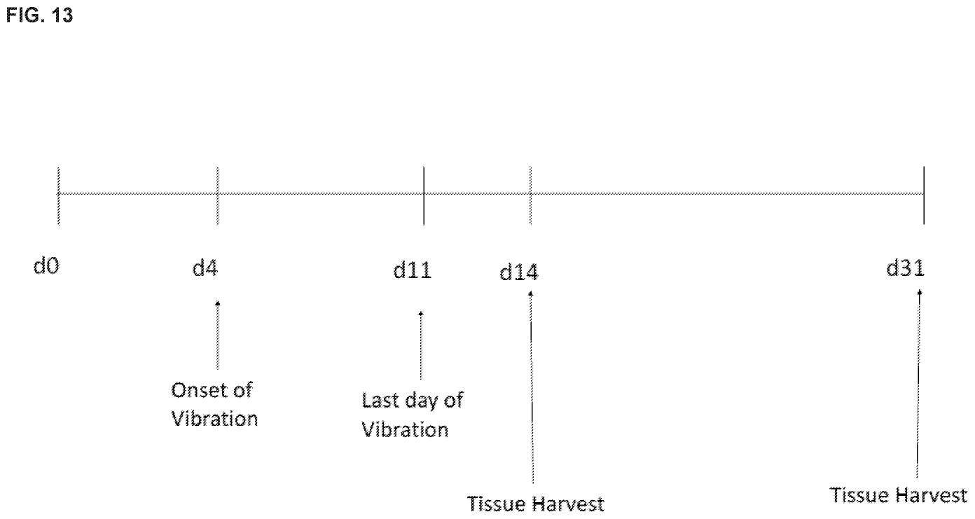

Altering tissue-scale forces around implants using mechanically stimulating implants (MSIs) produces human-like FBR capsule architecture in mice. To investigate the importance of allometric tissue-scale forces in FBR, it was examined whether altering mechanical forces in mice could produce a more human-like FBR. To do this, it would be needed to recreate the high mechanical stress environment, independent of implant size or chemistry. To accomplish this, an implantable device was developed encapsulating a small motor (FIG. 2B, FIG. 11-13), which could be induced to produce intermittent in situ implant vibration. This vibration was able to use the implant to intermittently increase the mechanical loading of the surrounding tissue. Finite element modeling predicted that these mechanically stimulating implants (MSIs) in mice would generate higher extrinsic forces from the surrounding host tissue and result in a 100-fold increase in mechanical stress at the implant-tissue interface (24.1 kPa), which is similar to that surrounding human implants (FIG. 2B). This provided a unique platform to examine the effect of the increased tissue-scale forces alone on the subsequent foreign body response.

When compared to standard implants, MS's developed FBR capsules with significantly increased collagen deposition, increased collagen maturity, and a higher level of activation of myofibroblasts, which was nearly identical to that of human clinical specimens (*p<0.05, FIG. 2CG). Analyzing the surface of murine and human implants by scanning electron microscopy, it was confirmed that the MS's and human implants were covered by a nearly identical and highly fibrous collagen network (*p<0.05, FIG. 2F,G). These findings demonstrate that by inducing high levels of extrinsic tissue-scale forces around murine implants, a human-like FBR capsule architecture can be recreated in mice, and this capsule is markedly different than that observed in standard murine implants.

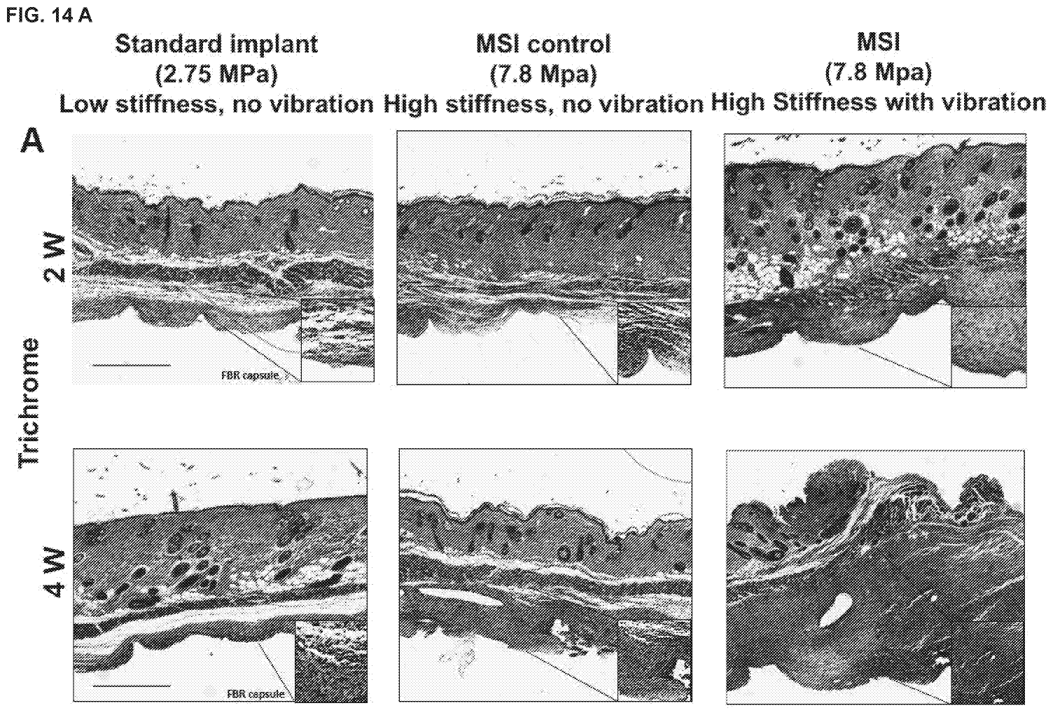

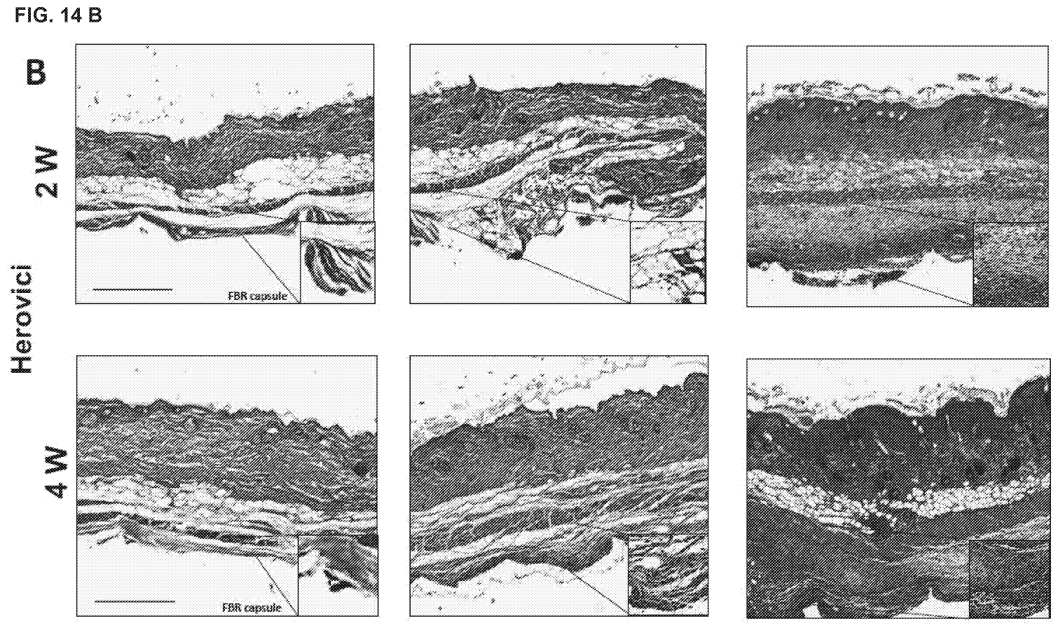

Both the MSI and standard implants were made of the same material (silicone) and had exactly the same geometry. To control for any differences resulting from the presence of the inactivated coin motor, additional experiments were performed to compare the FBR between MS's with the motor on and off (FIG. 14). It was found that the MS's without the motor activated were unable to generate a human-like highly fibrotic capsule (FIG. 14), confirming that the human-like FBR observed with the motor activated MS's (producing vibration) was entirely due to extrinsic tissue-scale forces. Thus, it was shown that increased extrinsic mechanical forces by the surrounding tissue results in a remarkably human-like highly fibrotic capsule, independent of both material chemistry and mechanical properties (FIG. 2, FIG. 14). Taken together, these results show for the first time that a third independent variable, the allometric tissue-scale forces, can drive the biology of FBR, independent of implant properties.

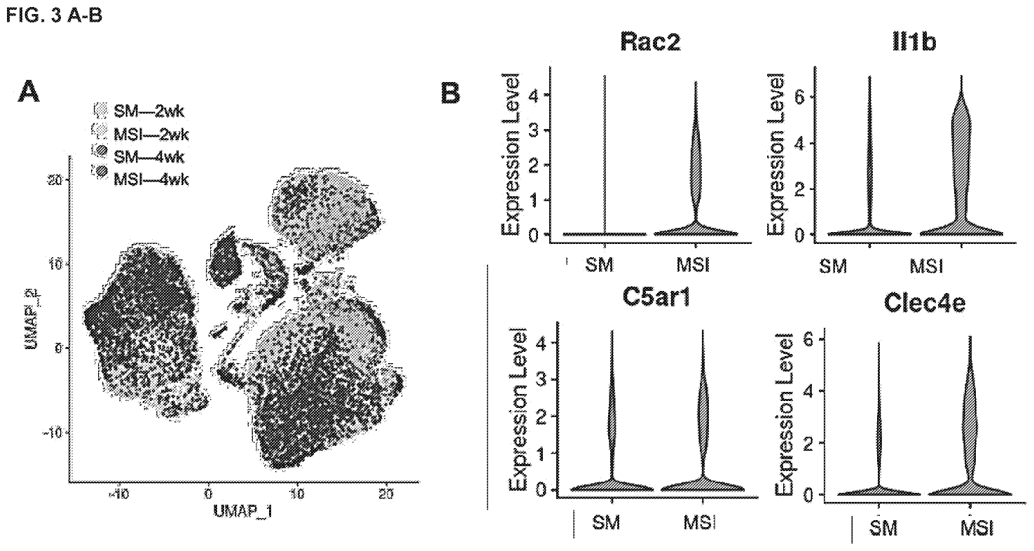

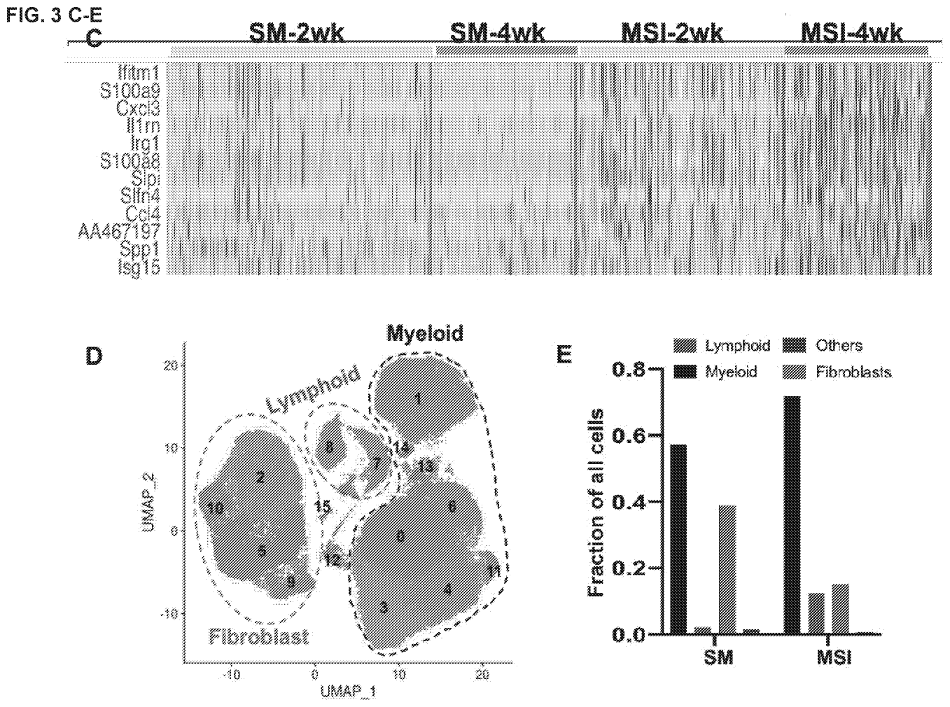

Elevated tissue-scale forces promote sustained activation of immune cell specific Rac2 mechanotransduction to drive FBR. To examine the cellular response through which extrinsic tissue-scale forces alter FBR, the cells surrounding the murine MSI were analyzed and standard implants using single cell RNA sequencing (scRNA-seq). A total of 36,827 cells were analyzed from both early-stage and late-stage capsules from both standard implants and MS's (FIG. 3A). These time points were chosen due to observable differences in the tissue architecture of the capsules as early as 2-weeks post implantation (early-stage), with the MSI capsules progressing to reach a stable human-like tissue architecture at about 4-weeks post-implantation (late-stage) (FIG. 2, FIG. 14).

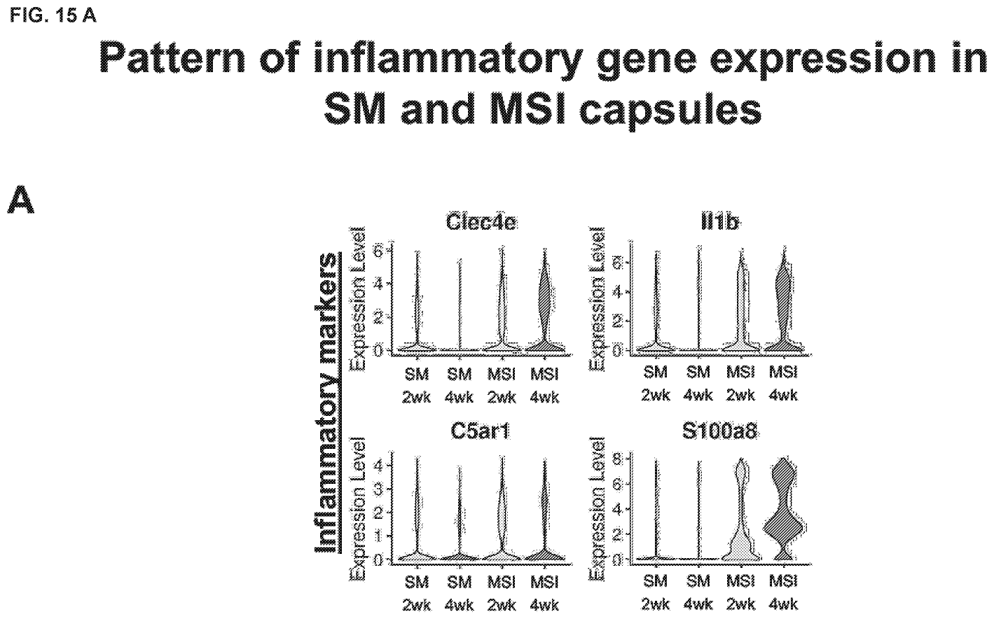

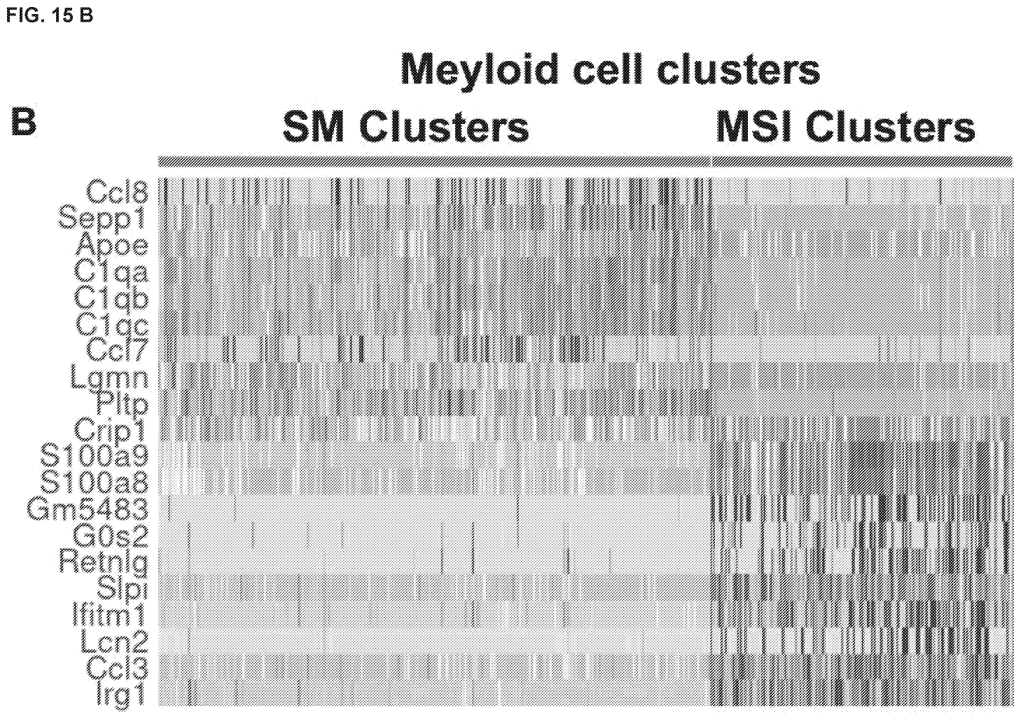

Analysis of the differential gene expression between cells isolated from standard implant capsules and MSI capsules revealed significant differences in the activation of Rac2 and associated inflammatory markers between the two groups. MSI cells showed a robust activation of Rac2 signaling in contrast to standard implant cells, which showed relatively little activation of Rac2 (FIG. 3B). Similarly, MSI cells showed a robust activation of inflammatory markers such as II1b, Clec4b, and C5ar1 (FIG. 3B). Standard implant cells initially expressed a modest activation of these inflammatory markers (II1b, Clec4b, and C5ar1), which subsided at later timepoints (FIG. 15). MSI capsules showed a robust early activation of these markers, which continued to increase in later time points. (FIG. 15). This pattern of expression was observed in most common inflammatory genes (FIG. 3C). The overall upregulation of Rac2 signaling and inflammation induced by mechanical stimulation is consistent with the highly inflammatory transcriptome of capsule tissue found in Baker IV specimen (FIG. 1).

To characterize the critical cell types that drive FBR, the SingleR automated cell identification software was employed to reveal two major cell types that drive FBR in both standard implants and MSIs: immune cells (myeloid and lymphoid), and fibroblasts (FIG. 3D). Myeloid cells, including monocytes, macrophages, dendritic cells, and granulocytes were the most abundant cell type in both the standard implant and MSI capsules (FIG. 3D). These myeloid cells were especially enriched with mechanical stimulation, confirming recent findings demonstrating that immune cells are involved beyond the acute phase of inflammation and may play a role in long-term FBR (FIG. 3E). The differential upregulation of inflammatory markers along with the increased presence of myeloid cells in MSI capsules further demonstrated that the human-like FBR in MS's is characterized by a mechanically-induced sustained inflammatory response at the implant-tissue interface (FIG. 3D,E).

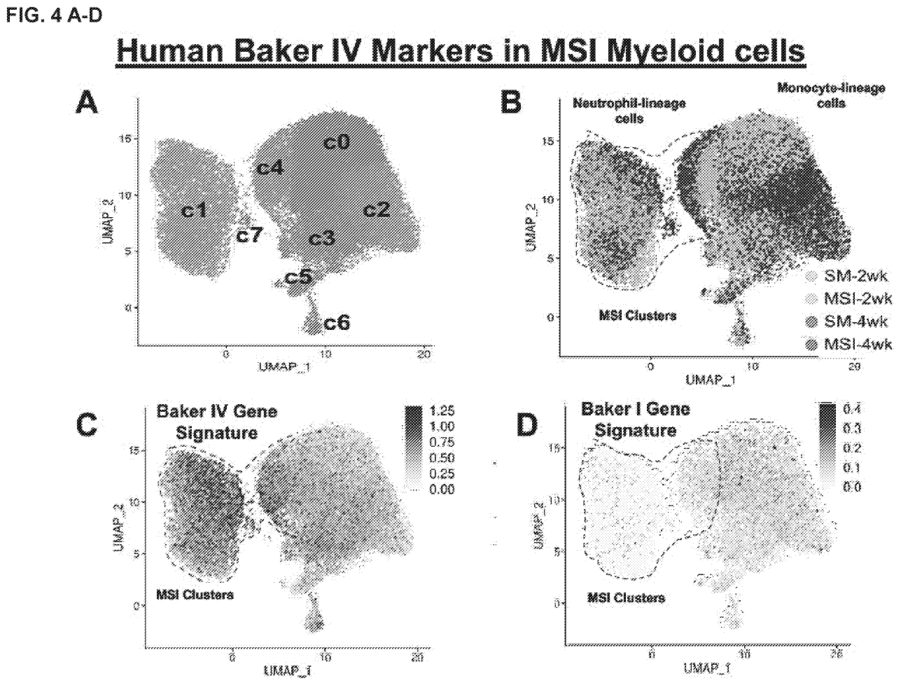

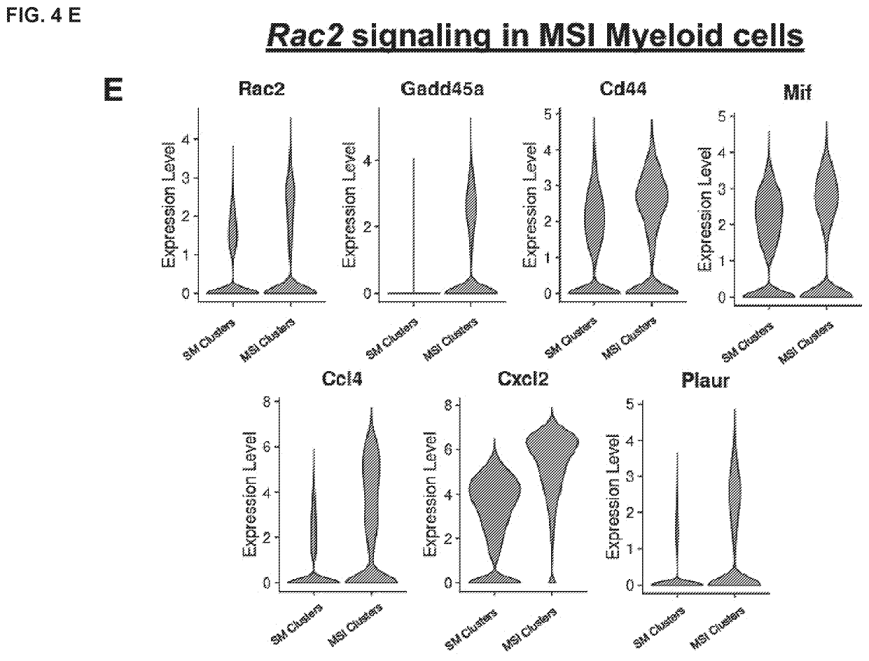

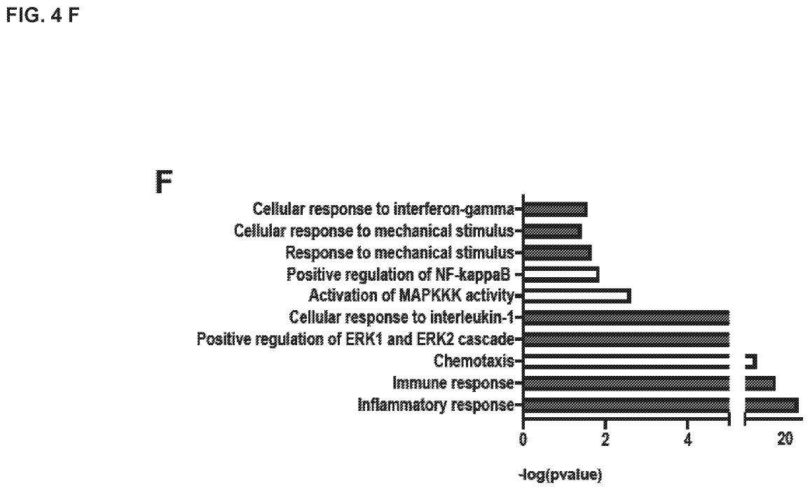

Rac2 immune signaling drives Baker IV FBR. Each cell type was subgrouped (i.e., myeloid, lymphoid and fibroblasts) for a detailed analysis of cell-type specific transcriptional shifts induced by increased levels of extrinsic tissue-scale forces. Myeloid cells clustered in 8 distinct subpopulations (Clusters 0-7, FIG. 4A). Of these myeloid cell clusters, Clusters 1 (granulocytes), 4 & 7 (macrophages) were specifically enriched in MS's (FIG. 4B).

Next, the transcriptional profiles of these murine clusters were examined and compared to the expression of Baker IV human biomarkers (FIG. 1), i.e. the genes that were highly upregulated in Baker-IV (severe) human specimens. The combined mean expression (mRNA) of the top 25 Baker-IV biomarkers, which include mechanical signaling genes and downstream inflammatory genes, represents a transcriptomic profile that drives pathological FBR and can be considered as a “human Baker IV gene signature”. The distribution of this “gene signature” was analyzed among myeloid cells from both standard implants and MS's using a combined gene expression feature plot (FIG. 4C,D). The human Baker IV gene signature was found to be highly upregulated in MSI clusters (Clusters 1,4,7) as compared to standard implant clusters (FIG. 4C). In contrast, standard implants did not show an upregulation of these genes, and instead upregulated genes found in the mild human Baker I capsules such as such as Slc25a4 and Endog (FIG. 4D), which indicate normal homeostatic processes.

Moreover, Rac2 and downstream mechanotransduction (e.g. Gadd45a, Mif, Cd44) and inflammatory genes (e.g. Cxcl2, Ccl4, Plaur) were differentially upregulated in the MSI clusters and human Baker IV capsules (FIG. 1, FIG. 4E, FIG. 15). DAVID pathway analyses further confirmed the significant overlap in the gene expression between MSI cells and human Baker IV capsules (FIG. 4F). Both human baker IV and murine MSI cells upregulated key mechanotransduction pathways, including “Positive regulation of Erk1 and Erk2 cascade” and “Cellular response to mechanical stimulus”, as well as inflammatory pathways, such as “Interferon-gamma signaling” and “Chemotaxis” (FIG. 4F).

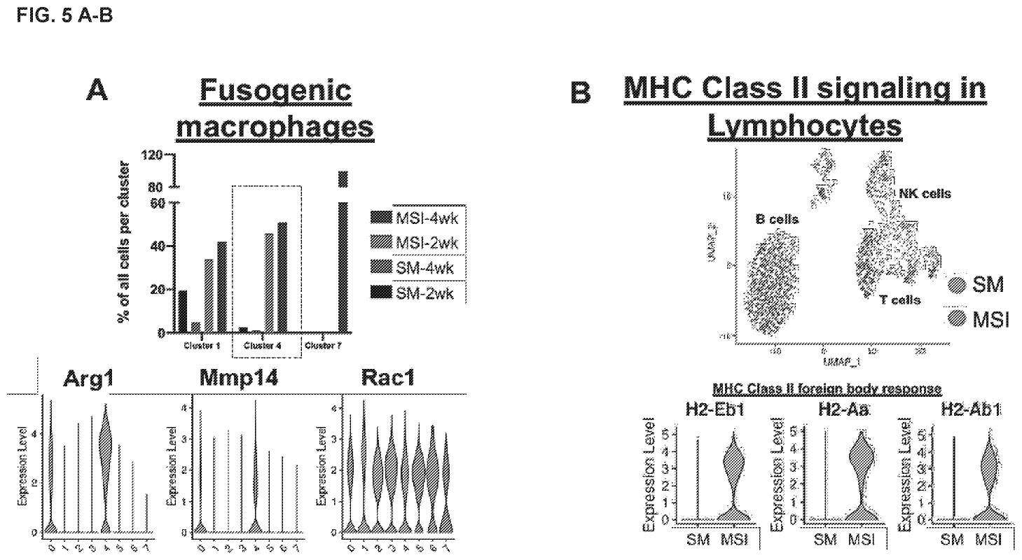

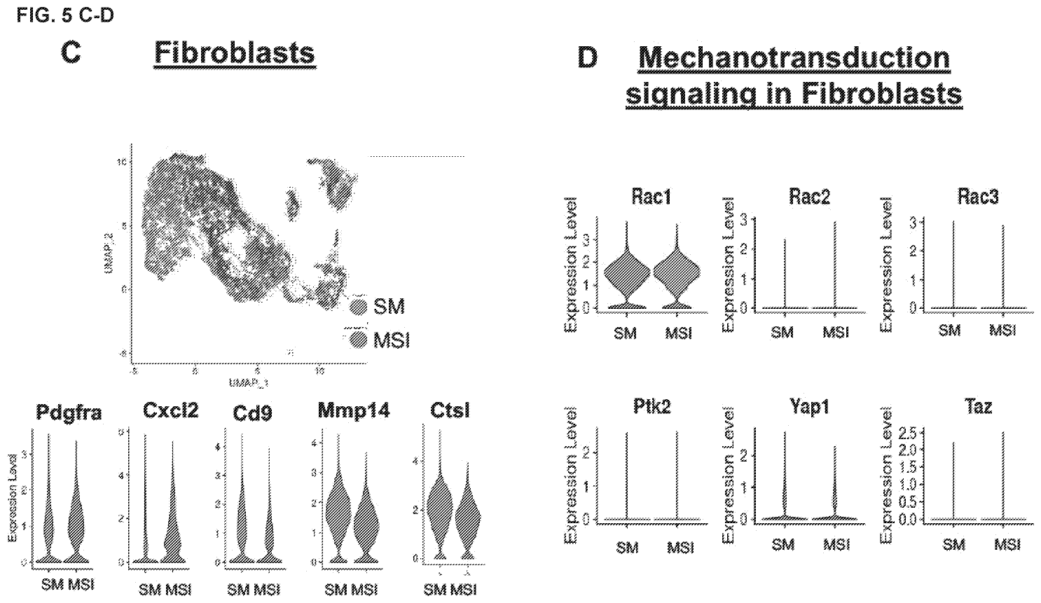

Extrinsic tissue-scale forces reproduce all classic features of pathologic FBR by activating fusogenic macrophages, MHC Class II lymphocytes and myofibroblasts. Fusion of macrophages leading to the formation of foreign body giant cells (FBGCs) is a hallmark of the classic FBR. Fusogenic macrophages and FBGCs are known to release degradative enzymes, ROS, and pro-fibrotic factors, which regulate the recruitment, growth and proliferation of fibroblasts. The exact molecular mechanism behind macrophage fusion in the presence of biomaterials is still a matter of debate. Arg1+ macrophages (Cluster 4), which have been previously reported to be fusogenic macrophages, were highly enriched in MSI capsules as compared to standard implants (FIG. 5A). Cluster 4 macrophages showed a simultaneous upregulation of Arg1, Rac1 and Mmp14 (FIG. 5A), demonstrating the activation of fusogenic macrophages in MS's in response to increased tissuescale forces at the implant-tissue interface.

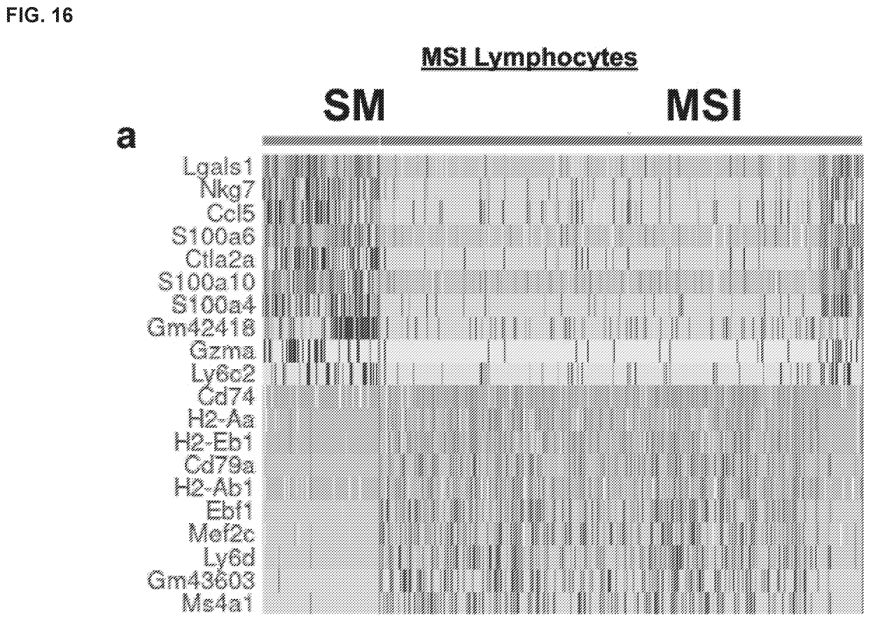

Since macrophage fusion is promoted by activated lymphocytes, all lymphocytes isolated from standard implants were subgrouped and MS's to identify the lymphoid-specific changes in transcriptional activity induced by mechanical stimulation. MSI lymphocytes showed a preferential upregulation of MHC class II foreign body response components including H2-Eb1, H2-Aa and H2-Aa1 (FIG. 5B, FIG. 16).

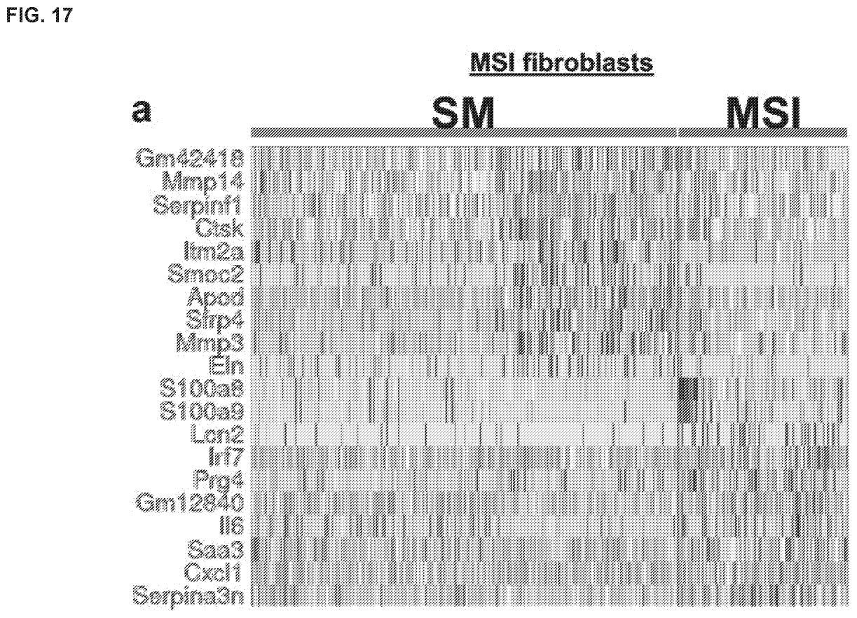

A substantial number of fibroblasts were found in both implants. Several inflammatory cytokines linked to the activation of fibroblasts such as Cxcl2, Plaur, and Ccl4, were upregulated in MSI capsules (FIG. 4, FIG. 15). Fibroblasts from both implant models were compared (FIG. 5C, FIG. 17), and found that fibroblasts in the standard murine model demonstrate upregulation of proteolysis (Mmp14, Ctsl) and negative regulation of cell proliferation (Cd9), suggesting a resolving phenotype. In contrast, MSI fibroblasts showed an upregulation of myofibroblast marker Pdgfra, profibrotic cytokines such as Cxcl2, and downregulation of anti-proliferation marker Cd9 (FIG. 5C), indicative of a more active fibroblast phenotype in MS's. This led to the increased differentiation of myofibroblasts and collagen deposition in MSI capsules, consistent with our observations of increased myofibroblasts populating the MSI capsules (FIG. 2). Interestingly, analysis of fibroblast mechanotransduction using Ptk2, Yap1, and Taz showed minimal activation of these pathways in either capsule. In addition, fibroblasts from MSI capsules and standard implant capsules displayed almost identical levels of Rac1 activation, and minimal activation of Rac2 and Rac3. The simultaneous activation of myofibroblast markers and minimal activation of mechanotransduction signaling components in MSI fibroblasts suggests that the activation of fibroblasts is primarily mediated by activated immune cells, rather than the extrinsic forces induced by implant vibration in vivo.

Blocking Rac2 signaling negates the effect of increased tissue forces, dramatically reducing FBR. The findings show that allometric tissue-scale forces activate Rac2 signaling in immune cells, which drive the classic human pathological FBR. Since the extrinsic tissue-scale forces are inherent to the size of the organism and cannot be altered, it would require pharmacological strategies to block mechanical activation of immune cells in FBR. To this end, the efficacy of a small molecule Rac inhibitor was tested (EHT 1864 2HCl) in reducing FBR in our MSI model (FIG. 6). Local injection of EHT 1864 2HCl in the MSI model reduced the expression of immune cell-specific Rac2 in the FBR capsules by about 80%, indicating a significant reduction in the recruitment and activation of mechanoresponsive immune cells (FIG. 6A). Correspondingly, a significant reduction was observed in the activation of myofibroblasts in MSI capsules treated with the small molecule inhibitor by about 90% (FIG. 6B). Blocking Rac2 signaling in mice significantly reduced the overall FBR as well, specifically demonstrated by decreased capsule thickness and collagen deposition (FIG. 6C-D). Taken together, these results show that blocking Rac2 signaling in immune cells can cause a cascade of downstream effects including decreased myofibroblast differentiation, decreased downstream collagen production, and significantly reduced FBR capsule formation. By blocking the immune orchestrators of FBR, it is possible to reverse the human-like FBR resulting from increased levels of extrinsic tissue-scale forces in mice.

Materials and Methods

Human implant capsule specimen. Explanted biomedical devices (breast tissue expanders and implants, neurostimulator batteries, pacemakers, and orthopedic implants) and the surrounding scar tissue were collected for this study and analyzed. Informed consent was obtained from each patient in accordance with the Institutional Review Board at Stanford University (IRB #41066).

Human tissue bank and RNA analysis. We employed a large tissue bank was employed for human breast implant capsule tissues, located in Regensburg, Germany, which consists of over 710 unique breast tissue samples. As relatively few patients with Baker I capsules undergo revisionary surgery, our overall sample size was limited by this group. 9 samples of Baker I capsules were identified in our biobank and this determined the sample size for this study (n=9 for Baker I samples, n=11 for Baker IV samples). The patients were of comparable ages: i) 40.6+/− 3.89 years at the time of implantation in Baker I and 35.8+/− 4.40 years in Baker IV, and ii) 50.3+/− 3.04 years at the time of explanation in Baker I and 51.0+/− 4.0 years in Baker IV. The patients had silicone breast implants placed for augmentation for a mean of 10.67+/− 2.79 years in Baker I and 15.23+/− 4.47 in Baker IV. None of the patients previously had cancer. For RNA analysis, 5 μm FFPE sections of human samples were lysed, proteinase K-digested, and analyzed by the HTG EdgeSeq qNPA assay (HTG Molecular Diagnostics, Tucson, AZ) using a biomarker panel (HTG Oncology Biomarker Panel), a 2,549-gene probeset, including markers for inflammation and fibrosis. Following EdgeSeq qNPA processing, samples were individually barcoded by Polymerase Chain Reaction (PCR) and pooled for sequencing. Libraries were sequenced on the Illumina NextSeq platform (Illumina, San Diego, CA) and data was processed with HTG's parser software. Approval was given by the local ethic committee in Regensburg (Reference No.: 15-101-0024). Differential expression analysis was performed with the EdgeR package in R (v3.14.0) with Benjamini-hochberg correction for multiple hypothesis testing. The 100 most highly ranked genes from this analysis for Baker I and Baker IV implants were used to perform gene set enrichment analysis against pathway databases using the Database for Annotation, Visualization and Integrated Discovery (DAVID) toolkit as described previously.

STRING analysis. To study the interaction of Rac2 with other Baker IV genes, STRING (Search Tool for the Retrieval of Interacting Genes/Proteins), a pathway analysis tool that predicts gene-gene interactions was employed as described before. The minimum required interaction score was set at 0.200. STRING analyzed interactions between the different genes based on experimental evidence as well as the predicted interactions. The relative positions of nodes and the distances between the different nodes are arbitrary. The gene-gene interactions, which are color-coded as listed below: pink (experimentally determined), blue (curated databases), green (gene neighborhood), red (gene fusion), dark blue (gene co-currence), light green (textmining), black (co-expression), violet (protein homology). Mice. All mice used in this study were housed in the Stanford University Veterinary Service Center and NIH and Stanford University animal care guidelines were followed. All procedures were approved by the university's Administrative Panel on Laboratory Animal Care. C57/BL6 wildtype mice (Jackson labs Stock No: 000664) were used in these experiments.

Implant fabrication. Standard silicone implants were made of polydimethylsiloxane (PDMS) and fabricated using a Sylgard 184 elastomer base and curing agent as previously described. A ratio of 5 (Elastomer):1 (Curing agent) was used for the experiments described. All implants were cylindrical in shape with a 1.55 cm diameter and 0.67±0.07 cm height. For mechanically stimulated implants, a prefabricated coin motor (Precision Microdevices) was placed in the elastomer solution before curing (FIG. 2b), while controls were PDMS alone. To enable in situ vibration of MSIs, the wires from the implant had to be guided through the skin, which required a novel surgical technique (FIG. 7). After skin incision and creation of a subcutaneous pocket on the back of the mice, two 20 G cannulas were inserted into the pocket in a cranio-caudal direction. The wires were tunneled through the pocket and guided through the skin using the cannulas and a modified Seldinger technique, enabling activation of the motor by an external battery. MS's could then be attached to the external battery for an hour every day during the fibroproliferative phase of FBR (Days 4-11), as outlined in FIG. 3a. Longer durations of vibration were not well-tolerated by mice. A 3V power source was chosen in accordance with our FE modeling to most accurately match the mechanical stress around implants in humans. As a second control, MSI implants (i.e. with coin motor) were used without in situ vibration.

Implant Mechanical Testing. The Young's Modulus (Ey) of silicone implants were determined using a custom compressive test method on Instron 5560 as described previously. Each sample had a diameter of 1.55 cm and subjected to a compressive rate of 1 mm/sec. Ey of each implant was calculated by taking the linear slope of the stress-strain curve between 0 and 0.10 compressive strain.

Implantation Experiments. Standard silicone implants and MS's were implanted in C57/BL6 mice for either 2 weeks or 1 month. A 2 cm incision was made on the dorsum of the mouse and a subcutaneous pocket was created. Control implants were placed in the subcutaneous pocket and the incision was closed using 6-0 nylon suture or staples. For vibration-enabled implants, the implants were placed into a subcutaneous pocket, similar to the procedure described above and wires were guided through the skin using a modified seldinger technique. 3V batteries with an amplitude of 1.38 G and a frequency of 203 Hz were used to mimic human conditions. MS's were vibrated for 1 hour daily from day 4 (D4) post implantation to day 11 (D11). This time period was chosen based on previous studies, showing that increased mechanical stress during this period effectively induces fibrosis.

Computational Modeling of biomechanical stress patterns around biomedical implants. Computational finite element (FE) models for human and mouse implants were developed using the commercial finite-element software ABAQUS (version 2017, SIMULIA, Providence, RI), using a similar FEM framework as previously described to study mechanical behavior of soft tissues as well as investigate deformation and stress patterns in biological tissues. Model geometry was based on experimental measurements of skin and fat layers and custom designed implants for humans and mice. (FIG. 7) In the initial configuration, the implants were modeled as a 3D disk. Fat, skin and muscle were represented as layers around the implant (see FIG. 7). Movement of implants transfers deformation and force to the layers of skin and fat. In all models, the bottom end of the muscle/bone layer was fixed. Tetrahedral elements (C3D4) were used for soft tissue layers, while hexagonal elements (C3D8) were used for implants. Mesh refinement confirmed that the chosen mesh size is accurate enough for the present purposes.

The simulation was based on the theory of elastic deformation of soft tissue where for each human and mouse model, different material properties were considered for skin, fat and muscle/bone layers based on previous findings (FIG. 9). The models contained external loading as static or vibrating forces, where the direction of applied force was in the horizontal axis in all models. Once the force was applied to implant, this force moves the 3D geometry of the implant in the direction of applied load. Due to the defined tie interaction between implant and tissue layers around it, implant movement applies the stress to the tissue where tissue on both ends of the implant experience negative (compression) and positive (stretch) stresses (see FIG. 2). The magnitude of these stresses are proportional to stiffness and elastic properties of the tissue. The stress experienced by tissue further triggers mechanotransduction pathways, which leads to biological responses. The models contained external loading as static or vibrating forces, where the direction of applied force was in the horizontal axis in all models. For human and mouse static models (see FIG. 1), the amplitude of the applied force was calculated from dynamic resting tensions reported previously. For the MSI model (see FIG. 2a), a periodic force was defined for vibrating implants, where the amplitude of the vibrating force was (F_static)_human=1.38 g≈13.5 N.

Histology and Trichrome staining. At each timepoint, the mice were euthanized, and the implants were resected en bloc with the surrounding scar tissue. The implants were removed and the scar tissue along with the skin was fixed in 4% paraformaldehyde overnight and embedded in paraffin. Scar tissue and implants from humans were collected from patients and processed within an hour. For analysis of the capsule, paraffin sections were stained with trichrome (SigmaAldrich) as described previously. Imaging was performed on a Leica DM5000 B upright microscope. Image analysis software (Image J) was used to quantify collagen staining.

Herovici's staining. Herovici's staining was performed according to manufacturer specifications as described below. Thin histological sections were deparaffinized and immersed in Weigert's hematoxylin solution for five minutes followed by Herovici's working solution (equal parts of stain solutions A and B) for 2 minutes. Slides were subsequently immersed in 1% acetic acid, followed by dehydration using alcohol and xylene washes. Finally, slides were mounted using mounting media with a coverslip on top. Imaging was performed on a Leica DM5000 B upright microscope. Image analysis software (Image J) was used to quantify mature collagen (red color) staining.

Scanning electron microscopy (SEM). Tissue on the surface of implants was fixed using 4% paraformaldehyde (PFA). Samples were dehydrated using a series of ethanol washes with increasing concentration from 70% to 100% ethanol for five mins each. The samples were subsequently immersed in hexamethyldisilazane for 15 min and then sputter coated with gold-palladium prior to imaging with SEM. For image analysis, at least eight different SEM images with collagen fibers on the surface of the implant were analyzed using imageJ for each group.

Immunostaining. Immunohistological staining was performed on paraffin sections as described previously. Briefly, heat-based antigen retrieval was followed by blocking with 5% goat serum in PBS. The following primary antibodies were used at a 1:200 dilution (as recommended by the manufacturer) and incubated overnight at 4° C.: anti-a-SMA (Abcam ab5694) or anti-Rac2 (Fischer Scientific, DF6273). Incubation of primary antibody-stained specimens with Alexa Fluor 488 secondary antibody (Thermo Fisher Scientific) was performed at a 1:400 dilution for 1 hour at room temperature. Sections were subsequently mounted using Fluoroshield (F6057, Sigma, Saint Louis, MO) with 4′, 6-diamidino-2-phenylindole (DAPI) to stain cell nuclei. Imaging was performed on a Leica DM5000 B upright microscope. Image analysis was performed using Matlab.

Single cell barcoding, library preparation, and single cell RNA sequencing. Implant capsule tissue were obtained from patients undergoing routine implant removal procedures, and was processed for single cell sequencing as described previously. Freshly obtained tissue from the clinic was micro-dissected and digested with collagenase to obtain cellular suspensions for 10× single cell sequencing (Single Cell 3′ v2, 10× Genomics, USA) according to the manufacturer's instructions. Briefly, a mixture of droplet-based single cell suspensions, partitioning oil and the reverse transcription master mix were loaded onto a single cell chip, and reverse transcription was performed on the Chromium controller at 53° C. for 45 mins. cDNA was amplified for 12 cycles on a BioRad C1000 Touch thermocycler using SpriSelect beads (Beckman Coulter, USA) and a ratio of SpriSelect reagent volume to sample volume of 0.6. cDNA was analyzed on an Agilent Bioanalyzer High Sensitivity DNA chip for qualitative control. cDNA was fragmented using the proprietary fragmentation enzyme blend at 32° C. for 5 min, which was followed by end repair and A-tailing for 30 min at 65° C. cDNA were double-sided size selected using SpriSelect beats, followed by ligation with sequencing adaptors at 20° C. for 15 min. cDNA amplification was performed using a sample-specific index oligo as primer, and subsequently another round of double-sided size selection using SpriSelect beads was performed. Final libraries were analyzed on an Agilent Bioanalyzer High Sensitivity DNA chip for quality control, and were sequenced using a HiSeq 500 IIlumina platform aiming for 50,000 reads per cell.

Data processing, FASTQ generation, and read mapping. The Cell Ranger software (10× Genomics; version 3.1)'s implementation of mkfastq was employed to convert base calls to reads. These reads were then aligned against the mm10 v3.0.0 genomes using Cell Ranger's count function (STAR v2.7.0) with SC2Pv2 chemistry and 5000 expected cells per sample. Cell barcodes representative of quality cells with i) at least 200 unique transcripts, and ii) less than 10% of their transcriptome of mitochondrial origin, were analyzed.

Data normalization and cell subpopulation identification. Raw Unique molecular identifiers or UMIs from each cell barcode were normalized with a scale factor of 10,000 UMIs per cell. The UMI reads were then natural log transformed with a pseudocount of 1 using the R package Seurat (version 3.1.1). Highly variable genes were identified, and cells were scaled by regression to the fraction of mitochondrial transcripts as described previously. The aggregated data was subsequently evaluated using uniform manifold approximation and projection (UMAP) analysis over the first 15 principal components and cell annotations were ascribed using SingleR toolkit (version 3.11) against the Immgen and mouse RNA-seq databases.

Generation of characteristic subpopulation markers and enrichment analysis. Seurat's native FindMarkers function with a log fold change threshold of 0.25 using the ROC test was used to generate marker lists for each cluster. The most highly ranked genes from this analysis were used to perform gene set enrichment analysis against pathway databases for each cluster or subgroup of cells using the Database for Annotation, Visualization and Integrated Discovery (DAVID) toolkit as described previously.

Rac Inhibition Experiments. EHT 1864 2HCl, a potent Rac family GTPase inhibitor, was acquired from Selleckchem (Houston, TX). MS's were implanted in C57/BL6 wildtype mice for 28 days utilizing the modified Seldinger technique described above. Mice were injected with EHT 1864 2HCl (10 mg/kg/day) (n=4) or saline (n=4) from Days 0-26. FBR capsule tissue was explanted on day 28 and processed for histologic analysis.

Statistical Analyses. Results are presented as mean±SEM. Standard data analysis was performed using student's ttests. ANOVA with posthoc tukey's test was used for multiple comparisons. Results were considered significant for *p<0.05.

### Example 2

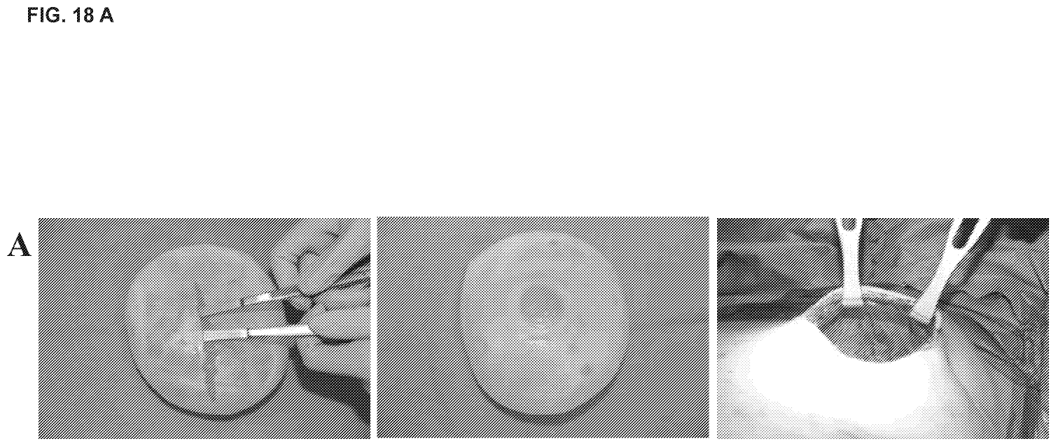

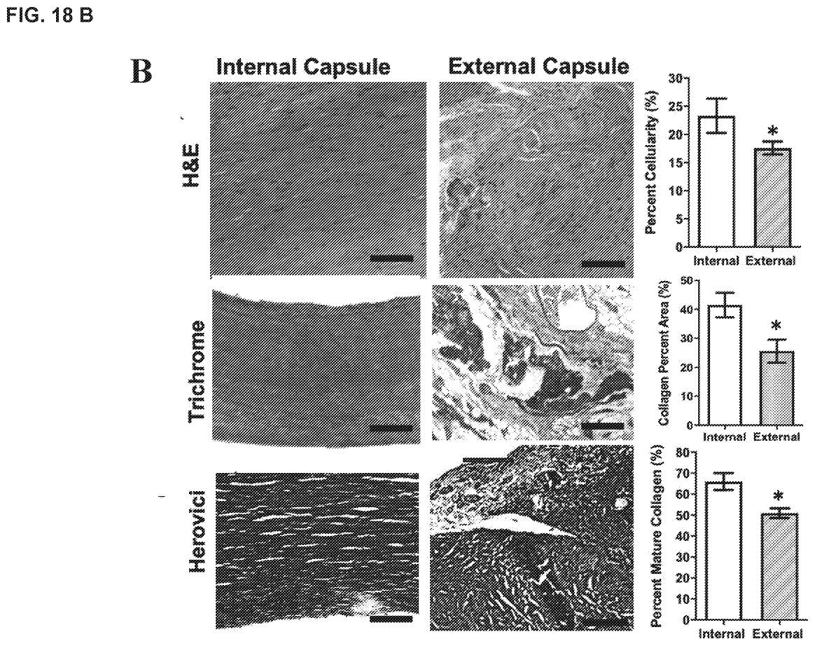

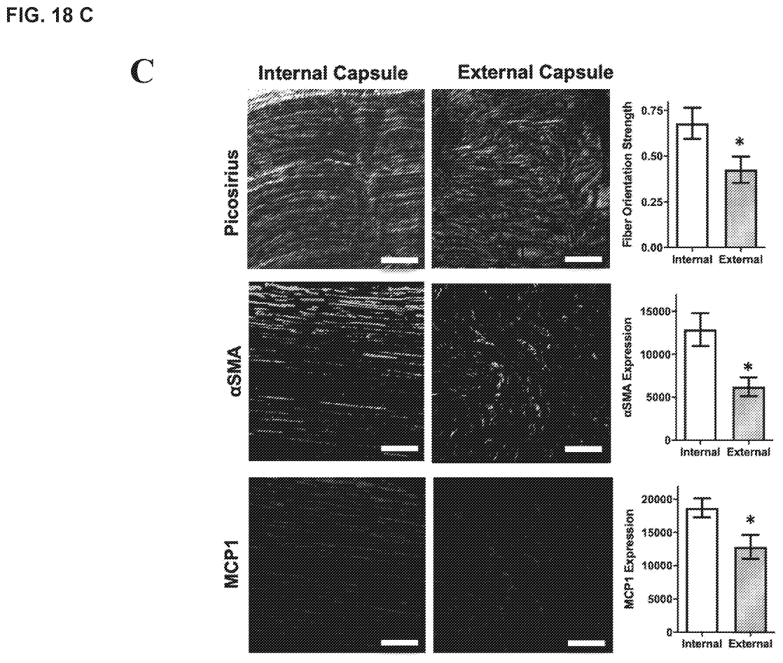

Taken together, these findings demonstrate that mechanical signaling is able to locally override genetic defects in collagen organization. I was found that the internal capsule was adherent to the underlying textured surface with a high coefficient of friction and mechanically constrained by the implant. The significant increase in the number of activated myofibroblasts and highly aligned collagen fibers, further confirmed the internal capsule was under elevated mechanical stress. In contrast, the external non-adherent capsule, displayed a loose collagen phenotype classic for Type I EDS. The absence of the internal capsule formation on the non-textured, smooth portion of the implant with identifying information tag and orientation knobs, provides additional support that the textured surface is directly responsible for the surprising presence of aligned collagen fibers in this EDS patient (FIG. 18D).

Finally, the increased expression of inflammatory signaling protein MCP-1 in the internal capsule is consistent with previous observations of mechanically-activated inflammation in fibrotic disease. Since prolonged inflammation around textured implants is universally considered to be a risk factor for ALCL these findings directly connect the textured surface of the implant to the pathogenesis of ALCL via the activation of mechanotransduction signaling.

In conclusion, the results show that textured implants with higher coefficients of friction activate mechanical signaling pathways and downstream inflammation to critically increase risk of ALCL. Thus, it is possible that no textured implant is completely “safe” for use. These data suggest that the textured surface is not an “innocent bystander” increasing biofilm or fragment deposition and may be causative in the pathogenesis of ALCL. To address these findings, mechanical signaling and inflammatory activation should be measured and used to quantitatively define the relative risk for ALCL for any given patient/implant-surface combination. These data provide a rational framework to quantify the risk for ALCL in individuals with textured implants that can guide future recommendations for revision surgeries in asymptomatic patients. These findings have significant implications of this work for changing the current clinical practice surrounding textured implants.

### Results

It seems likely that the two capsules resulted from the shearing of a single capsule at some point in the past, probably due to the COL5A genetic defect present in this patient's Type I EDS. Hematoxylin and eosin (H&E) staining of the internal capsule revealed that it was surprisingly organized and entirely comprised of aligned collagen fibers with increased cellularity (FIG. 18B, top). Trichrome staining demonstrated that this internal capsule was composed of thick fibrous tissue with increased collagen deposition (FIG. 18B, center). Herovici's stain, differentiating between mature and immature collagen, demonstrated predominantly mature collagen in the internal capsule (FIG. 18B, bottom), and Picrosirius red staining confirmed a highly aligned and tightly packed collagen fiber arrangement (FIG. 18C, top). This type of collagen fiber arrangement is dramatically different from the typical appearance of collagen deposition in EDS, which is characterized by loose, disorganized and wavy collagen fibers.

To confirm that the tight adherence of the internal capsule to the implant resulted in elevated mechanical stress, alpha-Smooth Muscle Actin (aSMA) was stained for, which identifies activated myofibroblasts commonly found in areas of scar contracture and fibrosis. A significant increase in myofibroblasts in the internal capsule was observed (FIG. 18C, center). Since elevated mechanical stress is associated with inflammatory signaling12, immunofluorescence staining for MCP1 was performed, an important inflammatory signaling molecule, and observed significantly higher expression in the internal capsule (FIG. 18C, bottom).

In contrast, the external capsule was comprised of loosely dispersed collagen fibers with observable alignment, which is more typical of Type I EDS (FIG. 18B,C).13 Immunofluorescence staining for myofibroblast marker aSMA and inflammatory cytokine MCP1 (FIG. 18C) were both markedly decreased, indicating lower levels of mechanical stress and inflammation.

### Materials and Methods

Here, data is presented that connects the textured implant surface directly to the increased risk for ALCL, raising questions about the safety of textured implants in general. An exceedingly rare case was reported of a textured implant revision surgery in a 59-year old woman with classical Ehlers-Danlos syndrome (EDS), which is a disease characterized by disorganized collagen fibers in connective tissues. The patient was concerned regarding the potential risk for BIA-ALCL given her textured implant and sought to exchange her textured implant for a smooth one. This patient was found to have one internal capsule tightly adherent to the implant, and a second external capsule attached to the subcutaneous tissue. Complete internal and external capsulectomy were performed and the capsule tissue architecture and molecular changes in the mechanically constrained internal capsule, and “free-floating” external capsule was analyzed using histology and immunohistochemistry.

### Example 3

As advances are made in materials sciences, electronics, and design of sophisticated biomedical devices, mitigating FBR to implantable materials remains a major challenge in developing biocompatible medical devices. The longevity of current biomedical implants is limited by adverse implant-tissue interactions, leading to implant failure and patient morbidity. As described previously, the lack of targeted therapeutic approaches to mitigate pathologic FBR is primarily a result of our incomplete understanding of the specific molecular and cellular mechanisms that underlie inflammatory responses to biomedical implants. One key component of fibrosis that has been previously overlooked in the study of FBR is mechanical signaling. It has recently shown that mechanical signaling is a key mediator of both hypertrophic scarring and FBR to implantable materials. Further, increased mechanical stress at the implant-tissue interface effectively recapitulates a human-like FBR capsule architecture in mice. Here, FBR capsules from both humans and MSI mice were investigated and mass spectrometry was utilized to identify upregulated pathways in FBR compared to control subcutaneous tissue. It was shown that IQGAP1 is elevated and serves as a common mediator of several of the upregulated mechanical signaling pathways present in both mice and human FBR capsules.

Previous literature on IQGAP1 has focused on its role in tumor progression, however its role in FBR has not been examined until now. IQGAP1 is a scaffolding protein that is associated with cell adhesion, cytoskeletal dynamics, and the cell cycle, all of which are disrupted in malignancy. Tumors have been portrayed as “wounds which do not heal” because they undergo continued stromal remodeling to ensure tumor propagation, a process similar to that of the proliferative resolution phase in wound healing. Similarly, FBR has been shown to initially begin as a wound healing response to local tissue damage that occurs during surgical implantation of a device, but subsequently transitions into a long-term response state involving fibrous capsule formation around the implant. FBR, in a sense, can be thought of as a “wound that never heals” due to the continued presence of the foreign implant material which prevents resolution of the wound healing response. Since the initial phase of FBR is essentially a wound healing response that follows surgical implantation, it is highly likely that mechanical signaling plays an important role in implant-tissue interactions during this process as well. Therefore, targeting proteins implicated in mechanical signaling like IQGAP1 represents a promising avenue for reducing excessive fibrosis around biomedical implants.

The data support the hypothesis that mechanical signaling plays an important role in fibrous encapsulation of implants. The importance of IQGAP1-mediated signaling was confirmed by employing a IQGAP1 deficient mouse model of FBR. Analyzing the RNA of single cells, it was demonstrated that IQGAP1 deficiency decreased transcription of mechanical signaling, inflammatory, and fibrosis related genes. Myeloid cells, which are involved in acute inflammation as well as chronic fibrosis, have recently been identified as being responsive to mechanical strain. The results show that IQGAP1 deficiency in macrophages lead to a diminished expression of Rac1, a Rho-GTPase, which has been shown to activate mechanical signaling in myeloid cells. Furthermore, it was shown that IQGAP1 deficiency promotes normal metabolic and adipogenic transcriptional programs in both myeloid cells and fibroblasts, as well as decreases inflammatory and myofibroblast transcriptional signatures, respectively. These transcriptomic findings were reflected in our histologic and immunochemical analyses, which revealed lower levels of collagen deposition, collagen maturity, myofibroblast activation, and activated Rac1 mediated mechanical signaling in IQGAP1 deficient mice. Taken together, these findings support an important role of IQGAP1 mechanical signaling networks in mediating pathologic inflammation and fibrosis in FBR.

Collectively, the results for the first time demonstrate that targeting mechanical signaling at the implant-tissue interface may improve the biocompatibility of biomedical implants, with broad implications for clinical application. A potential molecular target involved in pathologic FBR was investigated around biomedical implants and show that genetic deficiency of IQGAP1 ameliorates the effect of mechanically stimulated FBR by diminishing downstream mechanical signaling, inflammatory, and fibrotic pathways. Therefore, IQGAP1 may be a promising molecular target for rational therapeutic design to mitigate pathologic FBR.

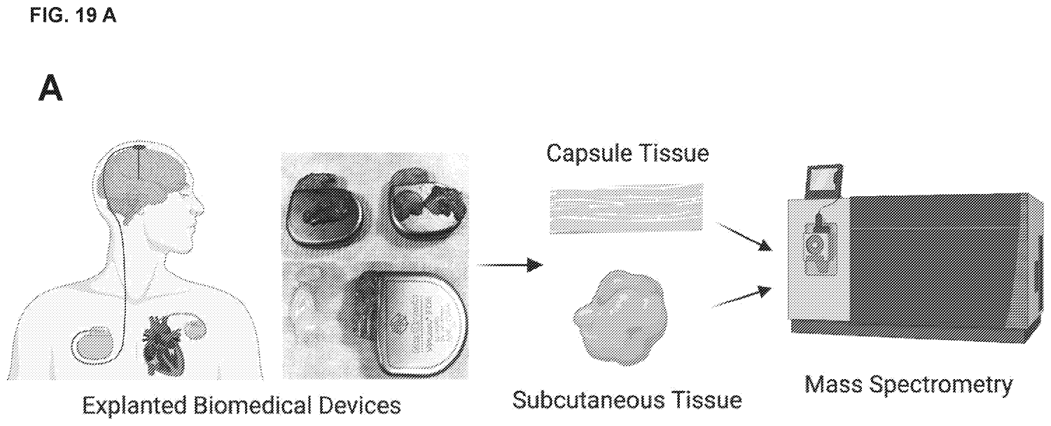

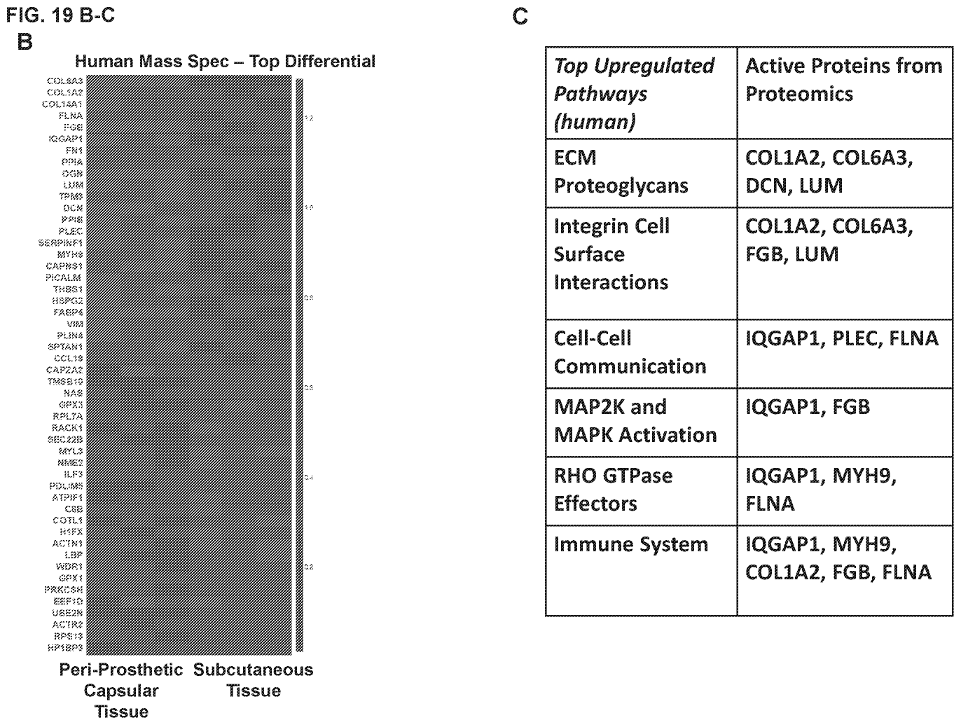

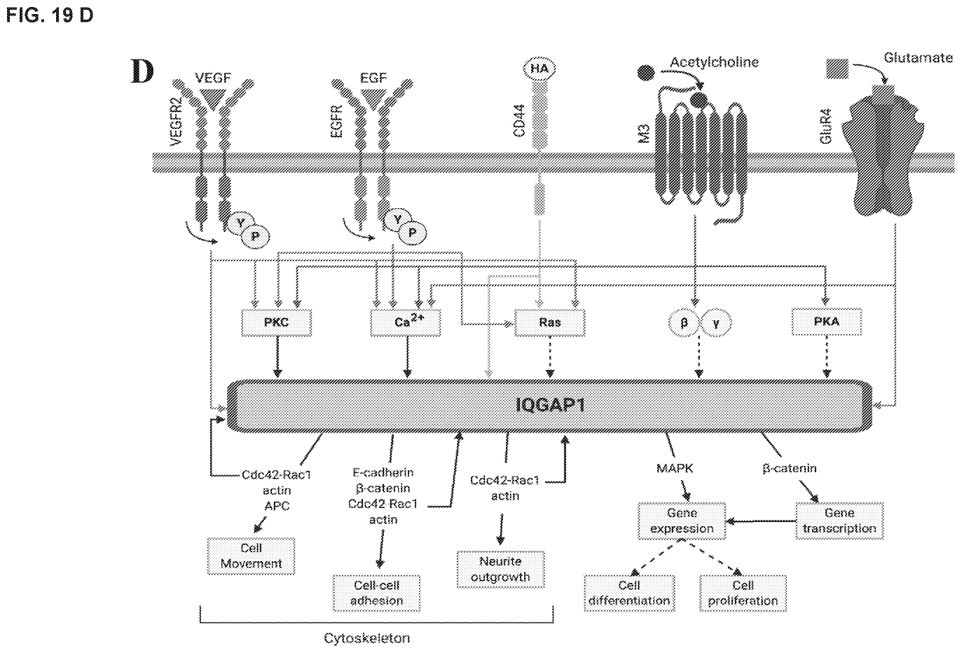

The IQGAP1 identified as a central mediator of mechanical signaling in human FBR. To determine the molecular mechanisms responsible for FBR, mass spectrometry was utilized to interrogate the protein content of human FBR capsules derived from biomedical implants (pacemaker and neurostimulator batteries) as well as human control subcutaneous tissue (FIG. 19a). Subcutaneous tissue was utilized as the control since most biomedical implants including breast implants, pacemaker batteries, neurostimulator batteries, continuous glucose monitoring (CGM) systems, and drug delivery devices are all implanted within the subcutaneous space. Proteins identified via mass spectrometry were analyzed using STRING (Search Tool for the Retrieval of Interacting Genes/Proteins), a pathway analysis tool that predicts protein-protein interactions. STRING analysis identified signaling pathways common to up-regulated proteins in human FBR capsule tissue compared to control subcutaneous tissue (FIG. 19b, c). This pathway analyses implicated mechanical signaling pathways such as MAPK and Rho-GTPase as well as ECM and integrin signaling pathways in FBR. Proteins COL1A2, FGB, MYH9, and IQGAP1 were present in several of the upregulated pathways. Collagen alpha-2 (COL1A2) chain is an important component of type 1 collagen, the collagen found in most connective tissues. Fibrinogen (FGB) has been shown to mediate cell adhesion via its role as an extracellular matrix protein, and myosin heavy chain 9 (MYH9) has been shown to promote activation of Rac1 GTPase upon phosphorylation. Interestingly, IQ motif containing GTPase Activating Protein (IQGAP1) was identified as a central, early-stage meditator common to several of the top upregulated pathways. IQGAP1 is a cytoplasmic scaffolding protein that has been previously described to be involved in multiple mechanical signaling pathways including beta-catenin, MAPK, and Rho-GTPase pathways, and plays an important role in connecting mechanical signaling with inflammation (FIG. 19d).

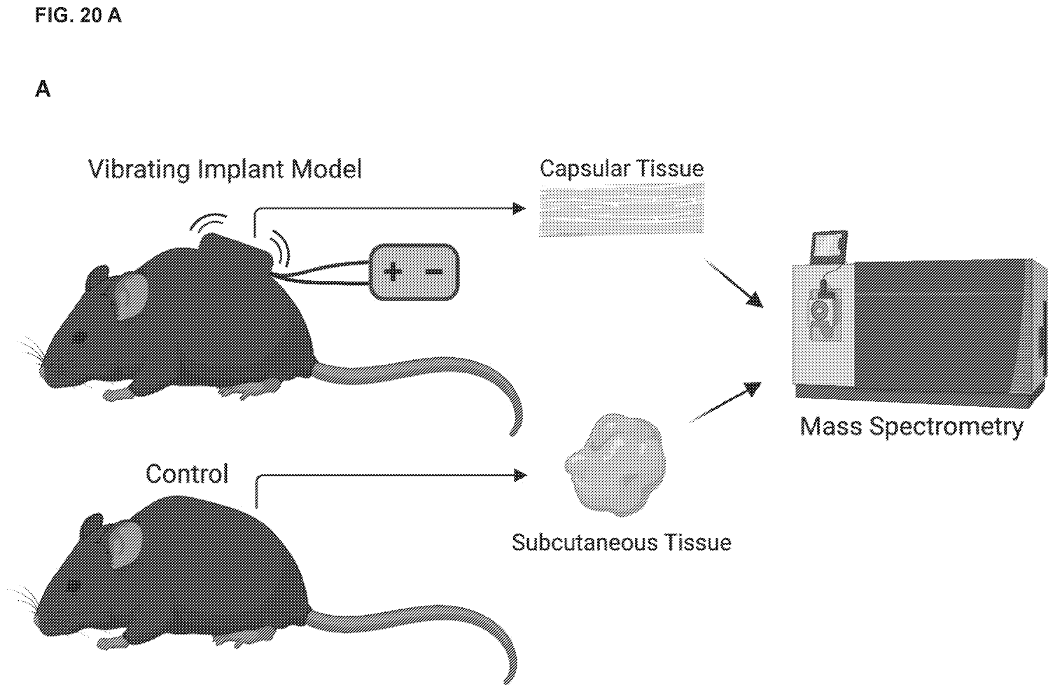

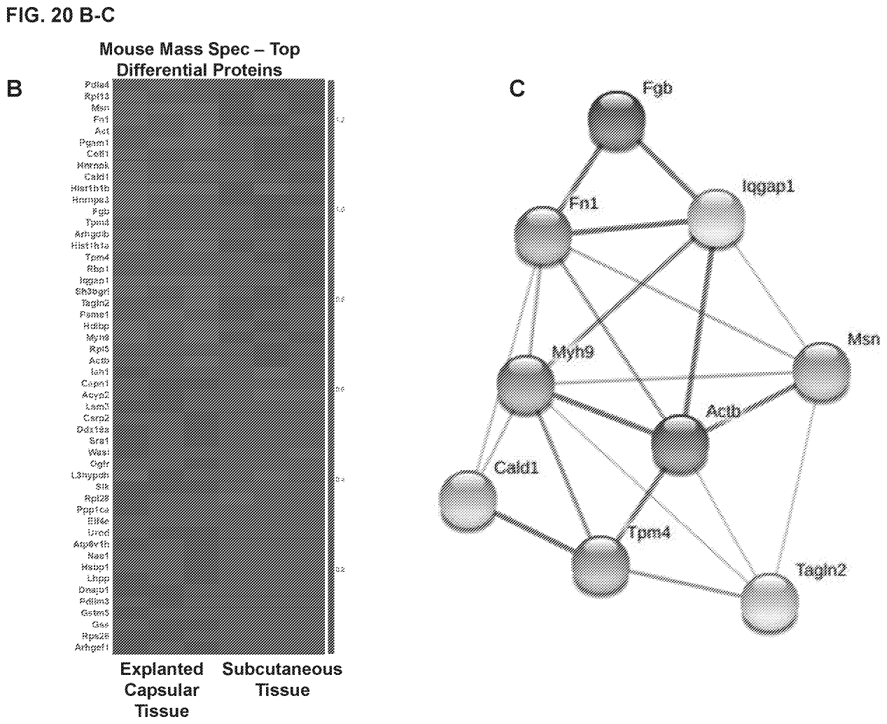

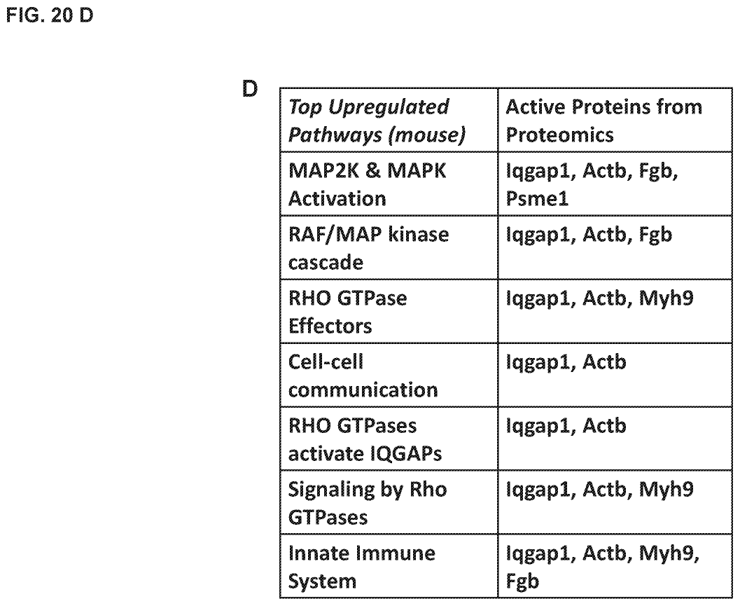

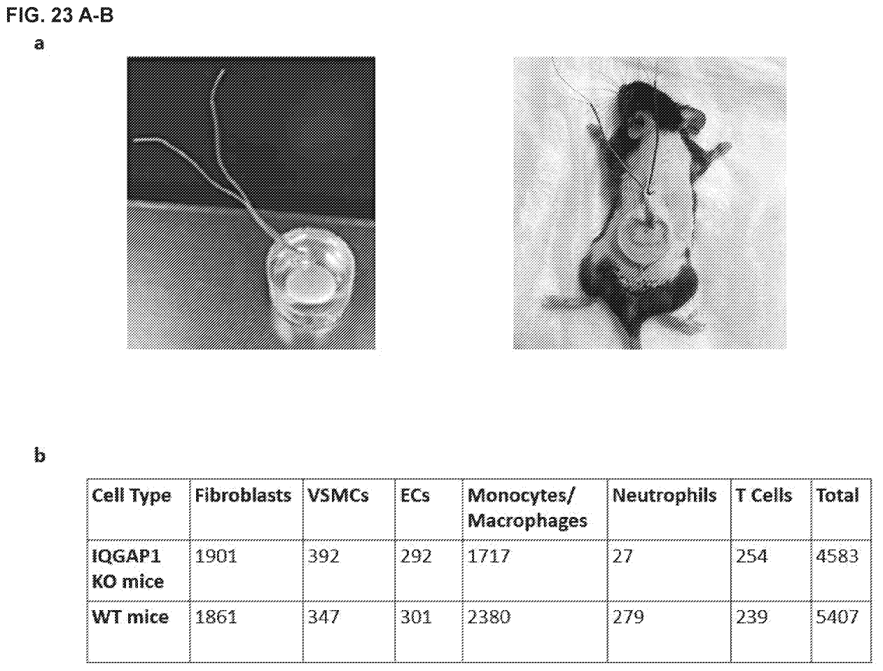

Proteomic analysis of FBR in mice confirms the activation of IQGAP1 mediated mechanical signaling pathways. To verify the role of IQGAP1 in FBR, mechanically stimulated implants (MSI) were utilized, which was previously shown to recapitulate human levels of mechanical stress in mice, to study FBR in a murine model (FIG. 23a). FBR capsules formed around MSI implants have been shown to produce a tissue architecture remarkably similar to what is observed in human FBR capsules with comparable collagen deposition, collagen maturity, and myofibroblast activation. The protein content of human-like MSI capsules were interrogated at two weeks following implant insertion, which is the timepoint at which the initial inflammatory response begins to progress into a fibrotic response. FBR capsules from the MSI models and murine control subcutaneous tissue were analyzed using mass spectrometry (FIG. 20a, b). Proteomics-based STRING pathway analyses of fibrous capsules from murine MSI identified multiple mechanical signaling pathways upregulated in MSI capsules, including the MAP2K, RAF/MAP kinase, and Rho-GTPase pathways. Similar to human FBR capsule tissue, IQGAP1 was identified as a common mediator of several of the mechanical signaling pathways upregulated in murine MSI capsules (FIG. 20c). Our proteomics and STRING analysis revealed agreement of upregulated mechanical signaling pathways and immune cell signaling pathways between MSI capsule tissue and human FBR capsule tissue (FIG. 20d). These data indicate that FBR to biomedical implants is critically regulated by mechanical signaling and that IQGAP1 serves as an important player in this process.

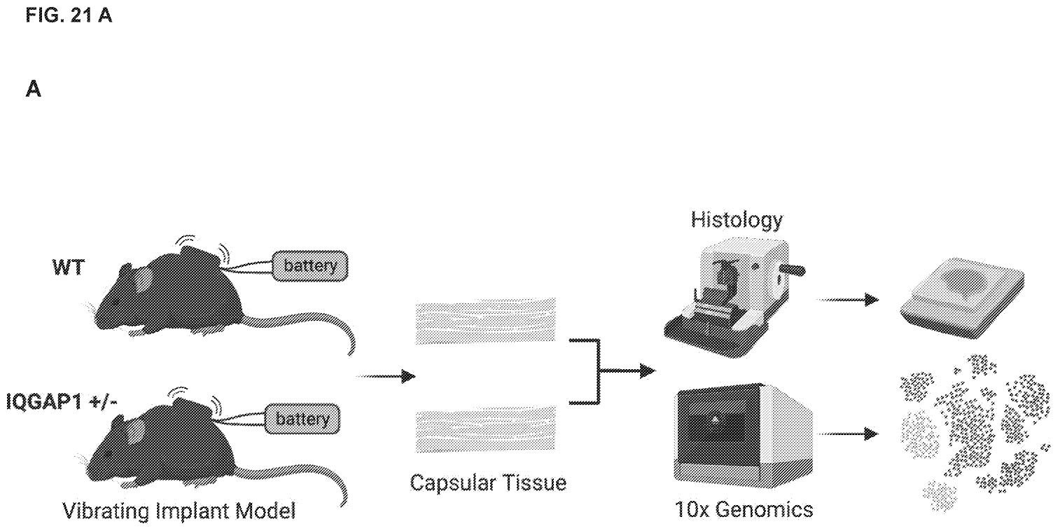

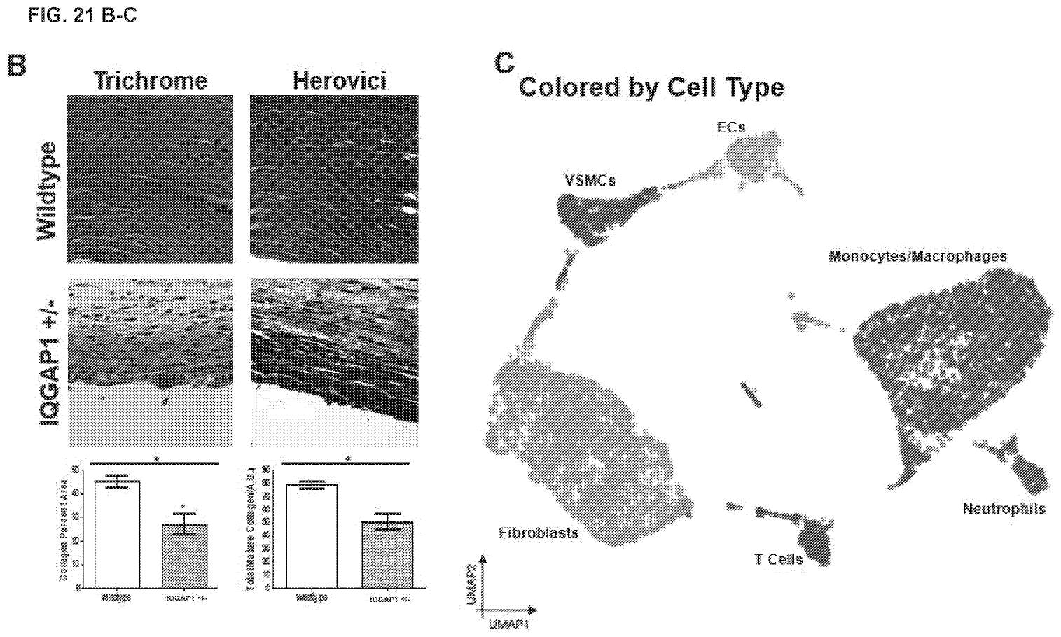

IQGAP1 deficient mice display decreased mechanical signaling, inflammation, and fibrosis. To verify the importance of IQGAP1-mediated signaling in FBR, the formation of fibrous capsules was examined around MS's in heterozygous mice (n=4) deficient for IQGAP1 (129-Iqgap1 tm1Aber/VsJ). As IQGAP1 KO mice have been previously shown to have a fragile phenotype prone to pulmonary vascular damage, IQGAP1+/− mice was used in order to prevent systemic complications from the significant mechanical forces imparted by MSI vibration. Histologic staining was performed to compare these capsules to those formed in wild-type (WT) mice (n=4) implanted with MS's (FIG. 21a). Compared to the capsules formed in WT mice with MS's, IQGAP1+/− mice displayed a significant reduction in fibrosis as evidenced by lower levels of collagen deposition and maturity on trichrome and herovici staining, quantified as total collagen percent area and total mature collagen, respectively (FIG. 21b).

Single cell RNA sequencing (scRNA-seq) was used utilizing the 10× Genomics Chromium platform to characterize the transcriptional signatures of cells involved in FBR capsule formation in both WT and IQGAP1+/− mice. A total of 9990 cells were captured, with 4403 myeloid cells, 3762 fibroblasts, 493 lymphoid cells, 739 vascular smooth muscle cells, and 593 endothelial cells identified using semi-supervised cell type annotations through the singleR package and evaluation of cell type specific marker genes (FIG. 21c, d) (FIG. 23b).

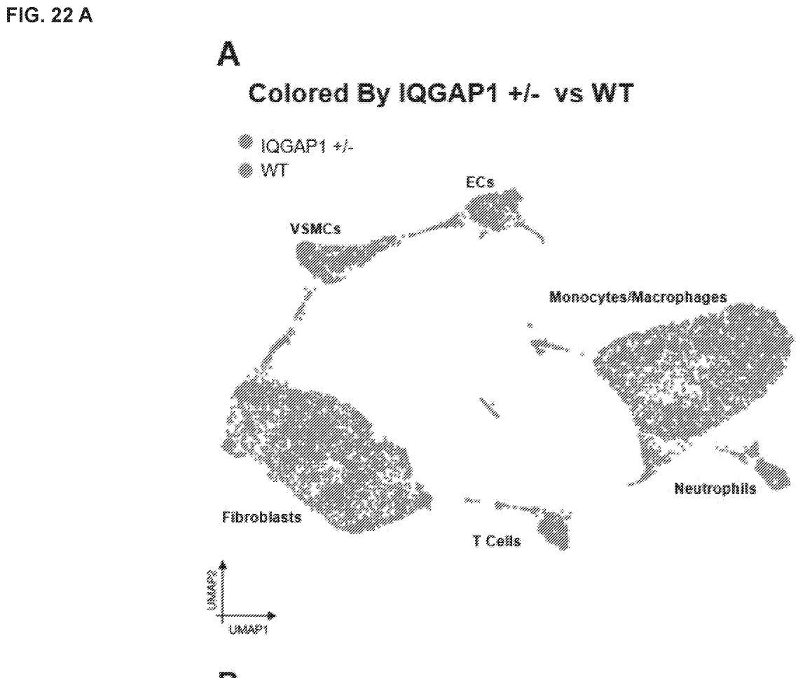

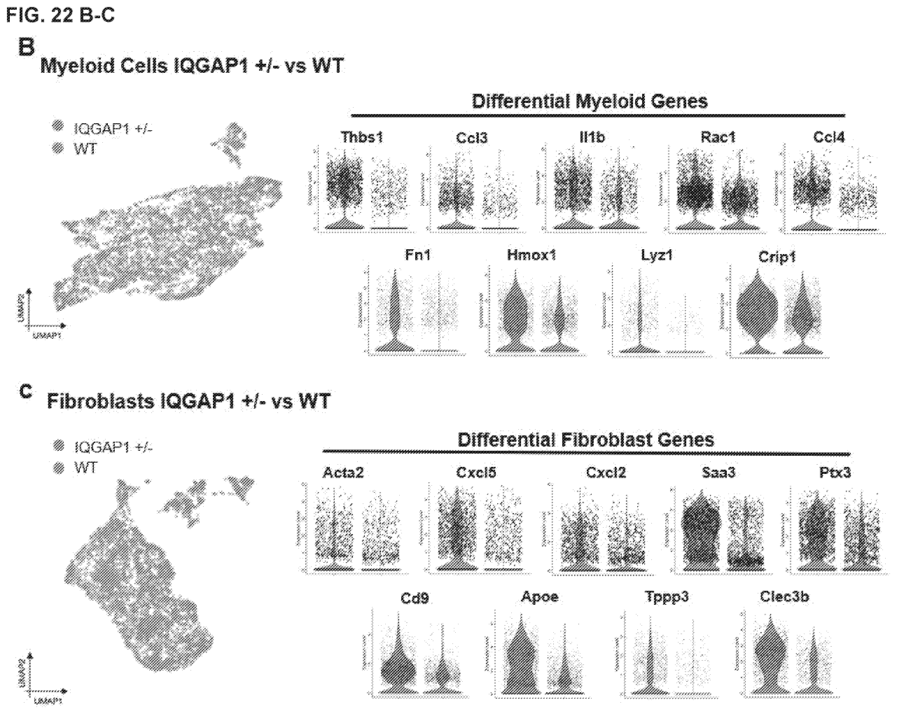

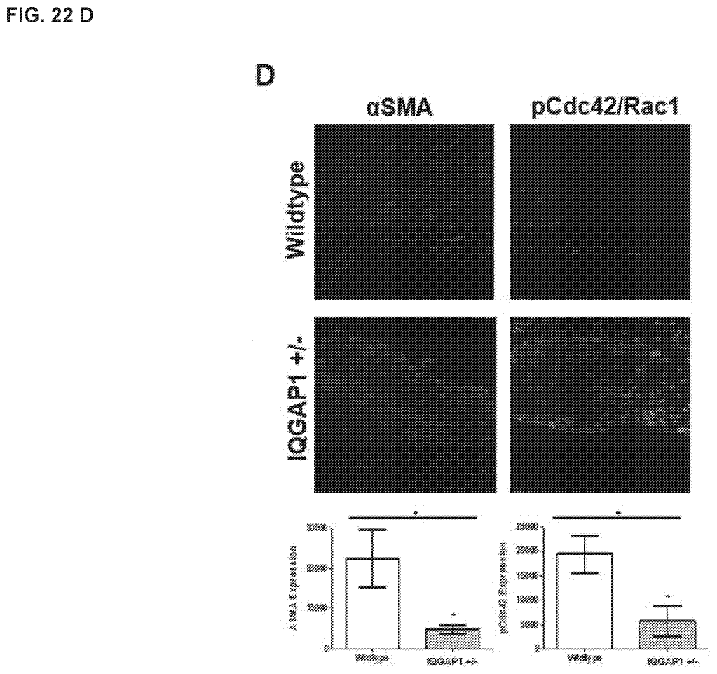

4583 IQGAP1+/− cells were captured and 5407 WT cells and observed that IQGAP1+/− cells demonstrated a clear shift in transcriptional programs (FIG. 22a). Given the increased numbers of fibroblasts and myeloid cells observed in the MSI capsule tissue, our single cell analysis was focused on these cell types. First, myeloid cell transcriptional profiles were compared between WT and IQGAP1+/− MSI capsule tissue. Interestingly, WT myeloid cells displayed increased Rac1 expression, a Rho-GTPase which is known to activate mechanical signaling and inflammation in myeloid lineage cells (FIG. 22b). We additionally observed that a series of genes associated with increased inflammatory responses were among the top differentially expressed genes in WT MSI capsule myeloid cells. Ccl3 and Ccl4, which are chemoattractants secreted by myeloid cells and are involved in recruiting other inflammatory and immune cells, were both upregulated in WT MSI capsule myeloid cells. These genes encode for macrophage inflammatory proteins (MIP) 1-alpha, and 1-beta respectively, both of which have been specifically linked to upregulating proinflammatory phenotypes. In WT capsule myeloid cells, MS's also upregulated Thbs1, a gene linked to macrophage activation and increased inflammation, as well as pro-inflammatory gene II-1β. Moreover, these inflammatory proteins Ccl3, Ccl4, II1b, and Thbs1 have all been previously shown to be transcriptionally upregulated by mechanical strain in myeloid cells. In IQGAP1+/− MSI capsule derived myeloid cells, several of the top differentially expressed genes have been associated with a phenotype indicative of normal metabolic processes. Specifically, Fn1, Hmox1, Lyz1, and Crip1 were all upregulated in IQGAP1+/− myeloid cells. Fn1 and Hmox1 have both been implicated cardiac and skeletal muscle repair, while Lyz1 and Crip1 have been associated with regeneration of paneth cells and neurons after injury, respectively.

Fibroblasts were examined in the WT and IQGAP1+/− MSI capsule tissue scRNA-seq dataset. WT fibroblasts were found to preferentially upregulate pro-fibrotic gene expression compared to those from IQGAP1+/− MSI capsules. Elevation of Acta2 was observed (encoding smooth muscle alpha actin, aSMA), as well as Cxcl5, Cxcl2, Saa3, and Ptx3, all of which have been implicated in fibrotic pathologies encompassing pulmonary, hepatic, and myocardial fibrosis (FIG. 22c). Specifically, the CXC-family of cytokines have been shown to positively influence myofibroblast phenoconversion and ECM deposition. These findings suggest that WT capsule fibroblasts have differentiated into a more contractile phenotype associated with increased collagen production and fibrosis. By contrast IQGAP1+/− MSI capsule fibroblasts were enriched for adipogenic markers Apoe and Clec3b, more characteristic of healthy subcutaneous tissue. Pro-regenerative genes Tppp3 and Cd9 were also among the top differentially expressed genes in IQGAP+/− fibroblasts. These genes have been previously linked to tissue repair of tendon and muscle, respectively.

Finally, immunostaining was utilized to confirm that IQGAP1 deficiency decreased mechanical signaling protein pcdc42 (activated Rac1). It was also confirmed that IQGAP1 deficiency decreased activation of myofibroblast marker aSMA (protein product of Acta2), indicating decreased capsule-associated myofibroblast activation and therefore a diminished fibrotic phenotype (FIG. 22d). Overall, these results demonstrate that IQGAP1 deficiency reduces mechanical signaling, inflammation, as well as myofibroblast activation, and instead promotes a more quiescent tissue phenotype.

### Materials and Methods

Human Tissue. Explanted biomedical devices (pacemaker and neurostimulator batteries) along with their surrounding fibrous capsule tissue were collected for this study and analyzed. Informed consent was obtained from each patient in accordance with the Institutional Review Board at Stanford University (IRB #41066).

Animals. All mice used in this study were housed in the Stanford University Veterinary Service Center and NIH and Stanford University animal care guidelines were followed. All procedures were approved by the university's Administrative Panel on Laboratory Animal Care.

Mechanically Stimulating Implant Fabrication. Mechanically stimulated polydimethylsiloxane (PDMS) implants were constructed by encapsulating prefabricated coin motors (Precision Microdevices) in a Sylgard 184 elastomer base and curing agent as previously described. A ratio of 5 (Elastomer):1 (Curing agent) was used for the experiments described. All implants were cylindrical in shape with a 1.55 cm diameter and 0.67±0.07 cm height.

In-Vivo Studies. MS's were implanted in C57/BL6 and IQGAP1+/− mice for 1 month. A 2 cm incision was made on the dorsum of the mouse and a subcutaneous pocket was created. The implants were placed into a subcutaneous pocket and wires were guided through the skin using a modified Seldinger technique, enabling activation of the motor by an external battery as described previously. MS's could then be attached to the external power source for an hour every day during the fibroproliferative phase of FBR (Days 4-11). 3V batteries with an amplitude of 1.38 G and a frequency of 203 Hz were used to mimic human levels of mechanical stress. MS's were vibrated for 1 hour daily from day 4 (D4) post implantation to day 12 (D11). This time period was chosen based on prior studies, which displayed that increased mechanical stress during this period effectively induces fibrosis.

Histology. After one month, all mice were euthanized, and the implants were resected en bloc with the surrounding scar tissue. The implants were removed and the scar tissue along with the skin was fixed in 4% paraformaldehyde overnight and embedded in paraffin. Scar tissue and implants from humans were collected from patients and processed within an hour of harvest. For analysis of the capsule, paraffin sections were stained with trichrome (SigmaAldrich) and herovici's (StatLab) staining as described previously. Imaging was performed on a Leica DM5000 B upright microscope. Image analysis software (Image J) was used to quantify collagen staining.

Immunostaining. Immunohistological staining was performed on paraffin sections as described previously. Briefly, heat-based antigen retrieval was followed by blocking with 5% goat serum in PBS. The following primary antibodies were used at a 1:200 dilution (as recommended by the manufacturer) and incubated overnight at 4° C.: anti-α-SMA (Abcam ab5694) or anti-p-cdc42 (Thermo Fischer 44-214 G). Incubation of primary antibody-stained specimens with Alexa Fluor 488 or Alexa Flour 597 secondary antibody (Thermo Fisher Scientific) was performed at a 1:400 dilution for 1 hour at room temperature. Sections were subsequently mounted using Fluoroshield (F6057, Sigma, Saint Louis, MO) with 4′, 6-diamidino-2-phenylindole (DAPI) to stain cell nuclei. Imaging was performed on a Leica DM5000 B upright microscope. Image analysis was performed using ImageJ. Six images were taken for each fibrous capsule sample, and average α-SMA and pcdc42 fluorescence pixel area per image was calculated and divided by the average number of cells within the image. Average number of cells were calculated using DAPI fluorescent stain and a MATLAB script previously used by the authors. Briefly, the image was converted to binary (fluorescent pixels=white, dark pixels=black) and analyzed white pixel clusters above a size area threshold of 50 pixels.

Mass Spectrometry. FBR capsules from explanted human biomedical devices, murine FBR capsules, and control human and murine subcutaneous tissue were all snap frozen and stored in liquid nitrogen. Frozen samples were digested and analyzed using liquid chromatography-based mass-spectrometry through the Stanford University Mass spectrometry (SUMS) core in order to generate the list of proteins upregulated in FBR capsules as compared to control subcutaneous tissue, in both humans and mice. Pathway analysis of the top upregulated proteins in human and murine FBR tissue was generated via STRING Functional Enrichment Analysis.

Single cell sequencing. Freshly obtained FBR capsule tissue were first micro-dissected and then enzymatically digested to obtain cellular suspensions of human fibroblasts for droplet-based microfluidic single cell RNA sequencing (scRNA-seq) using the 10× Chromium Single Cell platform (Single Cell 3′ v3, 10× Genomics, USA). Thousands of single cells from each type of FBR capsule were analyzed using 10× ScRNA-Seq. ScRNASeq data was analyzed using Seurat as described previously. Only cells with 10% or lower mitochondrial genes were considered for analyses. Subsequently the gene expression data was scaled, and the principal components were determined using linear dimensional reduction. Unbiased clustering of cells based on gene expression data using the principal components was performed. Non-linear dimensional reduction was applied to generate UMAP plots for data visualization and interpretation.

Statistical Analyses. Results are presented as mean±SEM. Standard data analysis was performed using student's t-tests. Results were considered significant for *p<0.05.

### REFERENCES

Kalorama Information, “The Global Market for Medical Devices,” (2019).
M. R. Major, V. W. Wong, E. R. Nelson, M. T. Longaker, G. C. Gurtner, The foreign body response: at the interface of surgery and bioengineering. Plast Reconstr Surg 135, 1489-1498 (2015).
E. B. Dolan et al., An actuatable soft reservoir modulates host foreign body response. Sci Robot 4, (2019).
M. T. Novak, F. Yuan, W. M. Reichert, Modeling the relative impact of capsular tissue effects on implanted glucose sensor time lag and signal attenuation. Anal Bioanal Chem 398, 1695-1705 (2010).
S. Mittal et al., Cardiac implantable electronic device infections: incidence, risk factors, and the effect of the AigisRx antibacterial envelope. Heart Rhythm 11, 595-601 (2014).
J. M. Anderson, A. Rodriguez, D. T. Chang, Foreign body reaction to biomaterials. Semin Immunol 20, 86-100 (2008).
A. J. Vegas et al., Combinatorial hydrogel library enables identification of materials that mitigate the foreign body response in primates. Nat Biotechnol 34, 345-352 (2016).
R. A. Bank, Limiting biomaterial fibrosis. Nat Mater 18, 781 (2019).
N. Noskovicova et al., Suppression of the fibrotic encapsulation of silicone implants by inhibiting the mechanical activation of pro-fibrotic TGF-beta. Nat Biomed Eng, (2021).
L. Zhang et al., Zwitterionic hydrogels implanted in mice resist the foreign-body reaction. Nat Biotechnol 31, 553-556 (2013).
V. Yesilyurt et al., A Facile and Versatile Method to Endow Biomaterial Devices with Zwitterionic Surface Coatings. Adv Healthc Mater 6, (2017).
J. Padmanabhan et al., In Vivo Models for the Study of Fibrosis. Adv Wound Care (New Rochelle) 8, 645-654 (2019).
J. Zhang et al., Molecular Profiling Reveals a Common Metabolic Signature of Tissue Fibrosis. Cell Reports Medicine 1, 100056 (2020).
J. Godec et al., Compendium of Immune Signatures Identifies Conserved and Species—Specific Biology in Response to Inflammation. Immunity 44, 194-206 (2016).
A. A. Biewener, Scaling body support in mammals: limb posture and muscle mechanics. Science 245, 45-48 (1989).
J. C. J. Wei et al., Allometric scaling of skin thickness, elasticity, viscoelasticity to mass for micro-medical device translation: from mice, rats, rabbits, pigs to humans. Scientific Reports 7, 15885 (2017).
J. R. Hutchinson, M. Garcia, Tyrannosaurus was not a fast runner. Nature 415, 1018-1021 (2002).
R. C. Rennert et al., A histological and mechanical analysis of the cardiac lead-tissue interface: implications for lead extraction. Acta Biomater 10, 2200-2208 (2014).
B. Kuehlmann, R. Burkhardt, N. Kosaric, L. Prantl, Capsular fibrosis in aesthetic and reconstructive-cancer patients: A retrospective analysis of 319 cases. Clin Hemorheol Microcirc 70, 191-200 (2018).
W. Huang da, B. T. Sherman, R. A. Lempicki, Systematic and integrative analysis of large gene lists using DAVID bioinformatics resources. Nat Protoc 4, 44-57 (2009).
V. W. Wong et al., Focal adhesion kinase links mechanical force to skin fibrosis via inflammatory signaling. Nat Med 18, 148-152 (2011).
S. Joshi et al., Rac2 is required for alternative macrophage activation and bleomycin induced pulmonary fibrosis; a macrophage autonomous phenotype. PLoS One 12, e0182851 (2017).
F. Y. McWhorter, C. T. Davis, W. F. Liu, Physical and mechanical regulation of macrophage phenotype and function. Cell Mol Life Sci 72, 1303-1316 (2015).
D. Szklarczyk et al., STRING v11: protein-protein association networks with increased coverage, supporting functional discovery in genome-wide experimental datasets. Nucleic Acids Res 47, D607-D613 (2019).
K. L. Helton, B. D. Ratner, N. A. Wisniewski, Biomechanics of the sensor-tissue interface-effects of motion, pressure, and design on sensor performance and the foreign body response-part I: theoretical framework. J Diabetes Sci Technol 5, 632-646 (2011).
K. Ren, R. Torres, Role of interleukin-1beta during pain and inflammation. Brain Res Rev 60, 57-64 (2009).
L. Backdahl, M. Aoun, U. Norin, R. Holmdahl, Identification of Clec4b as a novel regulator of bystander activation of auto-reactive T cells and autoimmune disease. PLoS Genet 16, e1008788 (2020).
N. Choudhry et al., The complement factor 5a receptor 1 has a pathogenic role in chronic inflammation and renal fibrosis in a murine model of chronic pyelonephritis. Kidney Int 90, 540-554 (2016).
D. Aran et al., Reference-based analysis of lung single-cell sequencing reveals a transitional profibrotic macrophage. Nat Immunol 20, 163-172 (2019).
J. C. Doloff et al., Colony stimulating factor-1 receptor is a central component of the foreign body response to biomaterial implants in rodents and non-human primates. Nat Mater 16, 671-680 (2017).
E. O. Apostolov, X. Wang, S. V. Shah, A. G. Basnakian, Role of EndoG in development and cell injury. Cell Death Differ 14, 1971-1974 (2007).
O. Lytovchenko, E. R. S. Kunji, Expression and putative role of mitochondrial transport proteins in cancer. Biochim Biophys Acta Bioenerg 1858, 641-654 (2017).
C. Rouault et al., Roles of chemokine ligand-2 (CXCL2) and neutrophils in influencing endothelial cell function and inflammation of human adipose tissue. Endocrinology 154, 1069-1079 (2013).
T. T. Chang, J. W. Chen, Emerging role of chemokine CC motif ligand 4 related mechanisms in diabetes mellitus and cardiovascular disease: friends or foes? Cardiovasc Diabetol 15, 117 (2016).
S. Thornton et al., Urokinase plasminogen activator and receptor promote collageninduced arthritis through expression in hematopoietic cells. Blood Adv 1, 545-556 (2017).
S. M. Jay, E. A. Skokos, J. Zeng, K. Knox, T. R. Kyriakides, Macrophage fusion leading to foreign body giant cell formation persists under phagocytic stimulation by microspheres in vitro and in vivo in mouse models. J Biomed Mater Res A 93, 189-199 (2010).
P. J. Brooks, M. Glogauer, C. A. McCulloch, An Overview of the Derivation and Function of Multinucleated Giant Cells and Their Role in Pathologic Processes. Am J Pathol 189, 1145-1158 (2019).
P. Gonzalo et al., MT1-MMP is required for myeloid cell fusion via regulation of Rac1 signaling. Dev Cell 18, 77-89 (2010).
Cordeiro, P. G. Breast Reconstruction after Surgery for Breast Cancer. New England Journal of Medicine 359, 1590-1601, doi:10.1056/NEJMct0802899 (2008).
Voelker, R. New Disease Cases, Deaths Related to Breast Implants. JAMA 324, 1277-1277, doi:10.1001/jama.2020.18305 (2020).
Collett, D. J. et al. Current Risk Estimate of Breast Implant-Associated Anaplastic Large Cell Lymphoma in Textured Breast Implants. Plast Reconstr Surg 143, 30S-40S, doi:10.1097/PRS.0000000000005567 (2019).
M, C. BIA-ALCL resources: by the numbers, and what they mean., <https://www.plasticsurgery.org/for-medical-professionals/health-policy/bia-alcl-physician-resources/by-thenumbers.>(2019).
FDA. (2019).
Pitenis, A. A. & Sawyer, W. G. Soft Textured Implants: Roughness, Friction, and the Complications.
Biotribology 22, 100127, doi:https://doi.org/10.1016/j.biotri.2020.100127 (2020).
Leberfinger, A. N. et al. Breast Implant—Associated Anaplastic Large Cell Lymphoma: A Systematic Review. JAMA Surgery 152, 1161-1168, doi:10.1001/jamasurg.2017.4026 (2017).
Hausser, I. & Anton-Lamprecht, I. Differential ultrastructural aberrations of collagen fibrils in Ehlers-Danlos syndrome types I-IV as a means of diagnostics and classification. Hum Genet 93, 394-407, doi:10.1007/BF00201664 (1994).
Bicca Ede, B., Almeida, F. B., Pinto, G. M., Castro, L. A. & Almeida, H. L., Jr. Classical Ehlers-Danlos syndrome: clinical, Histological and ultrastructural aspects. An Bras Dermatol 86, S164-167, doi:10.1590/s0365-05962011000700043 (2011).
Rao, K. B., Malathi, N., Narashiman, S. & Rajan, S. T. Evaluation of myofibroblasts by expression of alpha smooth muscle actin: a marker in fibrosis, dysplasia and carcinoma. J Clin Diagn Res 8, ZC14-17, doi:10.7860/JCDR/2014/7820.4231 (2014).
Rennert, R. C. et al. A histological and mechanical analysis of the cardiac lead-tissue interface: implications for lead extraction. Acta Biomater 10, 2200-2208, doi:10.1016/j.actbio.2014.01.008 (2014).
Wong, V. W. et al. Focal adhesion kinase links mechanical force to skin fibrosis via inflammatory signaling. Nat Med 18, 148-152, doi:10.1038/nm.2574 (2011).
Sobey, G. Ehlers-Danlos syndrome—a commonly misunderstood group of conditions. Clin Med (Lond) 14, 432-436, doi:10.7861/clinmedicine.14-4-432 (2014).
Padmanabhan, J. et al. In Vivo Models for the Study of Fibrosis. Adv Wound Care (New Rochelle) 8, 645-654, doi:10.1089/wound.2018.0909 (2019).
Myckatyn, T. M., Mehta-Shah, N. & Duncavage, E. Breast Implant-Associated Anaplastic Large Cell Lymphoma: Real, Rare, but Avoidable. JAMA Surgery 155, 3-4, doi:10.1001/jamasurg.2019.3154 (2020).

### Information, K. The Global Market for Medical Devices.

Liu, X., Zhou, L., Pan, F., Gao, Y., Yuan, X., Fan, D. Comparison of the postoperative incidence rate of capsular contracture among different breast implants: a cumulative meta-analysis. PLoS One 2015; 10:e0116071.
Mittal, S., Shaw, R. E., Michel, K., et al. Cardiac implantable electronic device infections: incidence, risk factors, and the effect of the AigisRx antibacterial envelope. Heart Rhythm 2014; 11:595-601.
Anderson, J. M., Rodriguez, A., Chang, D. T. Foreign body reaction to biomaterials. Semin Immunol 2008; 20:86-100.
Major, M. R., Wong, V. W., Nelson, E. R., Longaker, M. T., Gurtner, G. C. The foreign body response: at the interface of surgery and bioengineering. Plast Reconstr Surg 2015; 135:1489-1498.
Kastellorizios, M., Tipnis, N., Burgess, D. J. Foreign Body Reaction to Subcutaneous Implants. Adv Exp Med Biol 2015; 865:93-108.
Padmanabhan, J., Chen, K., Bonham, C. A., et al. Mechanical activation of inflammation at the implant-tissue interface underlies pathological foreign body response. (in review) 2021.
Duscher, D., Maan, Z. N., Wong, V. W., et al. Mechanotransduction and fibrosis. J Biomech 2014; 47:1997-2005.
Padmanabhan, J., Maan, Z. N., Kwon, S. H., Kosaraju, R., Bonham, C. A., Gurtner, G. C. In Vivo Models for the Study of Fibrosis. Adv Wound Care (New Rochelle) 2019; 8:645-654.
Agha, R., Ogawa, R., Pietramaggiori, G., Orgill, D. P. A review of the role of mechanical forces in cutaneous wound healing. J Surg Res 2011; 171:700-708.
Aarabi, S., Bhatt, K. A., Shi, Y., et al. Mechanical load initiates hypertrophic scar formation through decreased cellular apoptosis. FASEB J 2007; 21:3250-3261.
Wong, V. W., Rustad, K. C., Akaishi, S., et al. Focal adhesion kinase links mechanical force to skin fibrosis via inflammatory signaling. Nat Med 2011; 18:148-152.
Rinkevich, Y., Walmsley, G. G., Hu, M. S., et al. Skin fibrosis. Identification and isolation of a dermal lineage with intrinsic fibrogenic potential. Science 2015; 348:aaa2151.
Szklarczyk, D., Gable, A. L., Lyon, D., et al. STRING v11: protein-protein association networks with increased coverage, supporting functional discovery in genome-wide experimental datasets. Nucleic Acids Res 2019; 47:D607-D613.
Stuart, T., Butler, A., Hoffman, P., et al. Comprehensive Integration of Single-Cell Data. Cell 2019; 177:1888-1902 e1821.
Wenstrup, R. J., Cohn, D. H., Cohen, T., Byers, P. H. Arginine for glycine substitution in the triple-helical domain of the products of one alpha 2(1) collagen allele (COL1A2) produces the osteogenesis imperfecta type IV phenotype. J Biol Chem 1988; 263:7734-7740.
Sabbir, M. G., Dillon, R., Mowat, M. R. Dlc1 interaction with non-muscle myosin heavy chain 11-A (Myh9) and Rac1 activation. Biol Open 2016; 5:452-460.
Simpson-Haidaris, P. J., Rybarczyk, B. Tumors and fibrinogen. The role of fibrinogen as an extracellular matrix protein. Ann N Y Acad Sci 2001; 936:406-425.
Owen, D., Campbell, L. J., Littlefield, K., et al. The IQGAP1-Rac1 and IQGAP1-Cdc42 interactions: interfaces differ between the complexes. J Biol Chem 2008; 283:1692-1704.
Noritake, J., Watanabe, T., Sato, K., Wang, S., Kaibuchi, K. IQGAP1: a key regulator of adhesion and migration. J Cell Sci 2005; 118:2085-2092.
Urao, N., Razvi, M., Oshikawa, J., et al. IQGAP1 is involved in post-ischemic neovascularization by regulating angiogenesis and macrophage infiltration. PLoS One 2010; 5:e13440.
Hu, W., Wang, Z., Zhang, S., et al. IQGAP1 promotes pancreatic cancer progression and epithelial-mesenchymal transition (EMT) through Wnt/beta-catenin signaling. Sci Rep 2019; 9:7539.
Liu, J., Ni, X., Li, Y., et al. Downregulation of IQGAP1 inhibits epithelial-mesenchymal transition via the HIF1alpha/VEGF-A signaling pathway in gastric cancer. J Cell Biochem 2019; 120:15790-15799.
Roy, M., Li, Z., Sacks, D. B. IQGAP1 is a scaffold for mitogen-activated protein kinase signaling. Mol Cell Biol 2005; 25:7940-7952.
Swart-Mataraza, J. M., Li, Z., Sacks, D. B. IQGAP1 is a component of Cdc42 signaling to the cytoskeleton. J Biol Chem 2002; 277:24753-24763.
Bhattacharya, M., Su, G., Su, X., et al. IQGAP1 is necessary for pulmonary vascular barrier protection in murine acute lung injury and pneumonia. Am J Physiol Lung Cell Mol Physiol 2012; 303:L12-19.
Li, S., Wang, Q., Chakladar, A., Bronson, R. T., Bernards, A. Gastric hyperplasia in mice lacking the putative Cdc42 effector IQGAP1. Mol Cell Biol 2000; 20:697-701.
Hwaiz, R., Hasan, Z., Rahman, M., et al. Rac1 signaling regulates sepsis-induced pathologic inflammation in the lung via attenuation of Mac-1 expression and CXC chemokine formation. J Surg Res 2013; 183:798-807.
Provenzano, P. P., Keely, P. J. Mechanical signaling through the cytoskeleton regulates cell proliferation by coordinated focal adhesion and Rho GTPase signaling. J Cell Sci 2011; 124:1195-1205.
Wells, C. M., Walmsley, M., Ooi, S., Tybulewicz, V., Ridley, A. J. Rac1-deficient macrophages exhibit defects in cell spreading and membrane ruffling but not migration. J Cell Sci 2004; 117:1259-1268.
Maurer, M., von Stebut, E. Macrophage inflammatory protein-1. Int J Biochem Cell Biol 2004; 36:1882-1886.
Youn, B. S., Jang, I. K., Broxmeyer, H. E., et al. A novel chemokine, macrophage inflammatory protein-related protein-2, inhibits colony formation of bone marrow myeloid progenitors. J Immunol 1995; 155:2661-2667.
Liu, Z., Morgan, S., Ren, J., et al. Thrombospondin-1 (TSP1) contributes to the development of vascular inflammation by regulating monocytic cell motility in mouse models of abdominal aortic aneurysm. Circ Res 2015; 117:129-141.
Sid, B., Sartelet, H., Bellon, G., et al. Thrombospondin 1: a multifunctional protein implicated in the regulation of tumor growth. Crit Rev Oncol Hematol 2004; 49:245-258.
Chen, K., Henn, D., Sivaraj, D., et al. Mechanical Strain Drives Myeloid Cell Differentiation Toward Pro-Inflammatory Subpopulations. Advances in Wound Care (in review) 2021.
Wang, J., Karra, R., Dickson, A. L., Poss, K. D. Fibronectin is deposited by injury-activated epicardial cells and is necessary for zebrafish heart regeneration. Dev Biol 2013; 382:427-435.
Patsalos, A., Tzerpos, P., Halasz, L., et al. The BACH1-HMOX1 Regulatory Axis Is Indispensable for Proper Macrophage Subtype Specification and Skeletal Muscle Regeneration. J Immunol 2019; 203:1532-1547.
Liu, Y., Chen, Y. G. Intestinal epithelial plasticity and regeneration via cell dedifferentiation. Cell Regen 2020; 9:14.
Emes, R. D., Wang, W. Z., Lanary, K., Blackshaw, S. E. HmCRIP, a cysteine-rich intestinal protein, is expressed by an identified regenerating nerve cell. FEBS Lett 2003; 533:124-128.
Rockey, D. C., Weymouth, N., Shi, Z. Smooth muscle alpha actin (Acta2) and myofibroblast function during hepatic wound healing. PLoS One 2013; 8:e77166.
Strieter, R. M., Gomperts, B. N., Keane, M. P. The role of CXC chemokines in pulmonary fibrosis. J Clin Invest 2007; 117:549-556.
Siegmund, S. V., Schlosser, M., Schildberg, F. A., et al. Serum Amyloid A Induces Inflammation, Proliferation and Cell Death in Activated Hepatic Stellate Cells. PLoS One 2016; 11:e0150893.
Gorka-Dynysiewicz, J., Pazgan-Simon, M., Zuwala-Jagiello, J. Pentraxin 3 Detects Clinically Significant Fibrosis in Patients with Chronic Viral Hepatitis C. Biomed Res Int 2019; 2019:2639248.
Gharaee-Kermani, M., Kasina, S., Moore, B. B., Thomas, D., Mehra, R., Macoska, J. A. CXC-type chemokines promote myofibroblast phenoconversion and prostatic fibrosis. PLoS One 2012; 7:e49278.
Brzoska, E., Kowalski, K., Markowska-Zagrajek, A., et al. Sdf-1 (CXCL12) induces CD9 expression in stem cells engaged in muscle regeneration. Stem Cell Res Ther 2015; 6:46.
Harvey, T., Flamenco, S., Fan, C. M. A Tppp3(+)Pdgfra(+) tendon stem cell population contributes to regeneration and reveals a shared role for PDGF signalling in regeneration and fibrosis. Nat Cell Biol 2019; 21:1490-1503.
Huang, Z. H., Reardon, C. A., Mazzone, T. Endogenous ApoE expression modulates adipocyte triglyceride content and turnover. Diabetes 2006; 55:3394-3402.
Hoon, J. L., Tan, M. H., Koh, C. G. The Regulation of Cellular Responses to Mechanical Cues by Rho GTPases. Cells 2016; 5.
Rao, K. B., Malathi, N., Narashiman, S., Rajan, S. T. Evaluation of myofibroblasts by expression of alpha smooth muscle actin: a marker in fibrosis, dysplasia and carcinoma. J Clin Diagn Res 2014; 8:ZC14-17.
Goss-Varley, M., Dona, K. R., McMahon, J. A., et al. Microelectrode implantation in motor cortex causes fine motor deficit: Implications on potential considerations to Brain Computer Interfacing and Human Augmentation. Sci Rep 2017; 7:15254.
Dvorak, H. F. Tumors: wounds that do not heal-redux. Cancer Immunol Res 2015; 3:1-11.
Solis, A. G., Bielecki, P., Steach, H. R., et al. Mechanosensation of cyclical force by PIEZO1 is essential for innate immunity. Nature 2019; 573:69-74.

1. A method of preventing, inhibiting or treating an implant associated complication in an individual, the method comprising:
selecting an individual that has or is at risk of having an implant associated complication, and
administering to the individual an effective dose of a composition comprising a mechanotransduction inhibitor at or near the site of a biomedical implant.

2. The method of claim 1, wherein the implant associated complication is selected from foreign body response (FBR), implant-related cancers, fibrosis and capsular fibrosis.

3. The method of claim 2, wherein the implant-related cancer is breast implant associated anaplastic large cell lymphoma (BIA-ALCL).

4. The method of claim 1, wherein the mechanotransduction inhibitor is selected from an inhibitor of Rac Family Small GTPase 1 (RAC 1), RAC2, C—C Motif Chemokine Ligand 4 (CCL4), Growth and Arrest and DNA Damage Inducible Alpha (GADD45A), or IQ Motif Containing GTPase Activating Protein 1 (IQGAP1).

5. The method of claim 4, wherein the inhibitor of RAC1 is selected from NSC23766, ZINC69391, IA-166, or CAS 1177865-17-6 or the inhibitor of RAC2 is selected from NSC23766 or EHT 1684.

6. (canceled)

7.-8. (canceled)

9. The method of claim 1, wherein the administering step comprises implanting an implant comprising the composition.

10. The method of claim 1, wherein the composition prevents, inhibits, or reduces macrophage activity or differentiation, optionally Arg1⁺ macrophage activity or differentiation, by at least 10%, at least 20%, at least 30%, at least 40% or at least 50%.

11. (canceled)

12. The method of claim 1, wherein the composition prevents, inhibits, or reduces lymphocyte activity or differentiation by at least 10%, at least 20%, at least 30%, at least 40% or at least 50%.

13. The method of claim 1, wherein the composition prevents, inhibits, or reduces fibroblast activity or differentiation by at least 10%, at least 20%, at least 30%, at least 40% or at least 50%.

14. A biomedical device comprising an effective amount of a composition comprising a mechanotransduction inhibitor for preventing, inhibiting or treating an implant associated complication.

15. The biomedical device of claim 14, wherein the implant associated complication is selected from foreign body response (FBR), implant-related cancers, fibrosis or capsular fibrosis.

16. The biomedical device of claim 14, wherein the mechanotransduction inhibitor is selected from an inhibitor of Rac Family Small GTPase 1 (RAC 1), RAC2, C—C Motif Chemokine Ligand 4 (CCL4), Growth and Arrest and DNA Damage Inducible Alpha (GADD45A), or IQ Motif Containing GTPase Activating Protein 1 (IQGAP1).

17. The biomedical device of claim 14, wherein the device is coated with the composition.

18.-19. (canceled)

20. The biomedical device of claim 14, wherein the device is configured to administer the composition locally.

21. A small animal model for human-like foreign body response, the animal comprising:
a mechanically stimulating implant that produces intermittent in situ implant vibration and therein generates a human-like foreign body response.

22. The method of claim 1, wherein the composition prevents, inhibits, or reduces myeloid cell activity or differentiation, macrophage fusion or foreign body giant cell formation, by at least 10%, at least 20%, at least 30%, at least 40% or at least 50%.

23. (canceled)

24. A composition for use in the method of claim 1 comprising:
a mechanotransduction inhibitor and
a pharmaceutically acceptable excipient.

25.-27. (canceled)
